SEQUENCE LISTING (49 sequences)

SEQ ID NO: 1 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - DNA, 339 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 3 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 4 - DNA, 321 bp, Homo sapiens
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 5 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 6 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 7 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 8 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 9 - protein, 21 aa, Homo sapiens
MALPVTALLLPLALLLHAARP

SEQ ID NO: 10 - protein, 15 aa, Artificial Sequence
  source: /note=“Description of Artificial Sequence Synthetic peptide”
GGGGSGGGGSGGGGS

SEQ ID NO: 11 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 12 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 13 - protein, 622 aa, Homo sapiens [sequence omitted]

SEQ ID NO: 14 - protein, 230 aa, Artificial Sequence
  source: /note=“Description of Artificial Sequence Synthetic polypeptide”
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTSGQAG

SEQ ID NO: 15 - DNA, 693 bp, Artificial Sequence
  source: /note=“Description of Artificial Sequence Synthetic polynucleotide”
caggtgcagctgcaggagtccggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagaggggaagaatggggcttttgatatctggggccaagggacaatggtcaccgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactagtggccaggccggccac

SEQ ID NO: 16 - protein, 214 aa, Artificial Sequence
  source: /note=“Description of Artificial Sequence Synthetic polypeptide”
DIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 640 bp, Artificial Sequence
  source: /note=“Description of Artificial Sequence Synthetic polynucleotide”
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcagggttcagtggcagtggatctgggacagatttcactctcaccatcagcagtctgcaacctgaagattttgcaacttactactgtcaacagagttacagtaccccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctactgcctcagcagcaccctgacgctgagcaaagcagactacgagaaacacaaactctacgcctgcgaagtcacccatcagggcctgagctcgcccgtcacaaagagcttcaacaggggagagt

SEQ ID NO: 18 - protein, 214 aa, Artificial Sequence
  source: /note=“Description of Artificial Sequence Synthetic polypeptide”
RHQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 120 aa, Artificial Sequence
  source: /note=“Description of Artificial Sequence Synthetic polypeptide”
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSS

SEQ ID NO: 20 - protein, 262 aa, Artificial Sequence
  source: /note=“Description of Artificial Sequence Synthetic polypeptide”
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSSGGGGSGGGGSGGGGSRHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGQAGHHHHHHGDYKDDDDKG

SEQ ID NO: 21 - DNA, 801 bp, Artificial Sequence
  source: /note=“Description of Artificial Sequence Synthetic polynucleotide”
atggccttaccagtgaccgccttgctcctgccgctggccttgctgctccacgccgccaggccgcaggtgcagctgcaggagtccggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagaggggaagaatggggcttttgatatctggggccaagggacaatggtcaccgtctcttcaggtggaggcggttcaggcggaggtggctctggcggtggcggatcacgacatcagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgctcactttcggcggagggaccaaggtggagatcaaacggactgcggccgca

SEQ ID NO: 22 - DNA, 801 bp, Artificial Sequence
  source: /note=“Description of Artificial Sequence Synthetic polynucleotide”
atggcgctgccggtgaccgcgctgctgctgccgctggcgctgctgctgcatgcggcgcgcccgcaggtgcagctgcaggaaagcggcccgggcctggtgaaaccgagcgaaaccctgagcctgacctgcaccgtgagcggcggcagcgtgagcagcggcagctattattggagctggattcgccagccgccgggcaaaggcctggaatggattggctatatttattatagcggcagcaccaactataacccgagcctgaaaagccgcgtgaccattagcgtggataccagcaaaaaccagtttagcctgaaactgagcagcgtgaccgcggcggataccgcggtgtattattgcgcgcgcgaaggcaaaaacggcgcgtttgatatttggggccagggcaccatggtgaccgtgagcagcggcggcggcggcagcggcggcggcggcagcggcggcggcggcagccgccatcagatgacccagagcccgagcagcctgagcgcgagcgtgggcgatcgcgtgaccattacctgccgcgcgagccagagcattagcagctatctgaactggtatcagcagaaaccgggcaaagcgccgaaactgctgatttatgcggcgagcagcctgcagagcggcgtgccgagccgctttagcggcagcggcagcggcaccgattttaccctgaccattagcagcctgcagccggaagattttgcgacctattattgccagcagagctatagcaccccgctgacctttggcggcggcaccaaagtggaaattaaacgcaccgcggcggcg

SEQ ID NO: 23 - DNA, 801 bp, Artificial Sequence
  source: /note=“Description of Artificial Sequence Synthetic polynucleotide”
atggccctcccggtaacggctctgctgcttccactcgcactgctcttgcatgctgccagaccacaggtccagctgcaggagagtgggcctggactggttaagccgagtgagacactttccttgacgtgcactgtgagcgggggaagtgtgtcctcaggtagttattactggtcctggattcgccagccaccaggaaagggactggagtggataggttatatctattattctggcagcactaattacaatccttctctcaaaagtagggtgacaatttcagtggatacttccaaaaatcagtttagtctgaagctcagctctgtgacagctgctgatactgcagtttactactgcgccagggaggggaagaatggcgccttcgatatttggggacagggcactatggtgactgtatcaagcggaggcggtggcagcggcgggggagggagtggaggcggcgggtctcgacatcagatgacacagagcccatcatcacttagcgccagcgttggcgaccgggttacgataacatgcagggcttcccaatctatcagttcttatctgaactggtatcagcagaaaccaggtaaggcccccaagctgctcatctacgcagcctcatccctgcagagcggcgtccctagtcgattttccggtagtgggtcagggacagattttaccctgactatcagttcactgcagcccgaggacttcgcgacatactattgccaacagtcctatagtacacccttgacatttggcggcgggactaaagtagaaattaaacgcaccgcggccgca

SEQ ID NO: 24 - protein, 10 aa, Artificial Sequence
  source: /note=“Description of Artificial Sequence Synthetic peptide”
GGSVSSGSYY

SEQ ID NO: 25 - protein, 7 aa, Artificial Sequence
  source: /note=“Description of Artificial Sequence Synthetic peptide”
IYYSGST

SEQ ID NO: 26 - protein, 12 aa, Artificial Sequence
  source: /note=“Description of Artificial Sequence Synthetic peptide”
AREGKNGAFDIW

SEQ ID NO: 27 - protein, 6 aa, Artificial Sequence
  source: /note=“Description of Artificial Sequence Synthetic peptide”
QSISSY

SEQ ID NO: 28 - protein, 4 aa, Artificial Sequence
  source: /note=“Description of Artificial Sequence Synthetic peptide”
AASS

SEQ ID NO: 29 - protein, 10 aa, Artificial Sequence
  source: /note=“Description of Artificial Sequence Synthetic peptide”
QQSYSTPLTF

SEQ ID NO: 30 - protein, 21 aa, Homo sapiens
MALPVTALLLPLALLLHAARP

SEQ ID NO: 31 - protein, 21 aa, Homo sapiens
MALPVTALLLPLALLLHAARP

SEQ ID NO: 32 - protein, 21 aa, Homo sapiens
MALPVTALLLPLALLLHAARP

SEQ ID NO: 33 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 34 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 35 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 36 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 37 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 38 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 39 - protein, 287 aa, Homo sapiens
MSPHPTALLGLVLCLAQTIHTQEEDLPRPSISAEPGTVIPLGSHVTFVCRGPVGVQTFRLERDSRSTYNDTEDVSQASPSESEARFRIDSVREGNAGLYRCIYYKPPKWSEQSDYLELLVKESSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 40 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 41 - protein, 181 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 42 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 43 - protein, 244 aa, Artificial Sequence
  source: /note=“Description of Artificial Sequence Synthetic polypeptide”
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQAAAPTTTPAPRPPTPAPTIASQPLSLRPEACRPAAGGAVHTRGLDFACDIYIWAPLAGTCGVLLLSLVITLYCNHRRIQ

SEQ ID NO: 44 - protein, 23 aa, Artificial Sequence
  source: /note=“Description of Artificial Sequence Synthetic peptide”
GSGATNFSLLKQAGDVEENPGPM

SEQ ID NO: 45 - protein, 236 aa, Artificial Sequence
  source: /note=“Description of Artificial Sequence Synthetic polypeptide”
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 46 - protein, 502 aa, Artificial Sequence
  source: /note=“Description of Artificial Sequence Synthetic polypeptide”
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQAAAPTTTPAPRPPTPAPTIASQPLSLRPEACRPAAGGAVHTRGLDFACDIYIWAPLAGTCGVLLLSLVITLYCNHRRIQGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 47 - DNA, 1726 bp, Artificial Sequence
  source: /note=“Description of Artificial Sequence Synthetic polynucleotide”
accggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactggccaccatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccaggcggccgcacccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaggcggatccaaggatctggagcaacaaacttctcactactcaaacaagcaggtgacgtggaggagaatcccggccccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaactcgag

SEQ ID NO: 48 - protein, 5 aa, Artificial Sequence
  source: /note=“Description of Artificial Sequence Synthetic peptide”
SGGGS

SEQ ID NO: 49 - protein, 4 aa, Artificial Sequence
  source: /note=“Description of Artificial Sequence Synthetic peptide”
YVKM

(19) United States
(12) Patent Application Publication
(10) Pub. No.: US 2020/0010803 A1
(43) Pub. Date: Jan. 9, 2020

(54) IMMUNE CELL COMPOSITIONS AND METHODS OF USE

(71) Applicant: Memorial Sloan Kettering Cancer Center, New York, NY (US)

(72) Inventor: Prasad S. Adusumilli, New York, NY (US)

(73) Assignee: Memorial Sloan Kettering Cancer Center, New York, NY (US)

(21) Appl. No.: 16/491,140

(22) PCT Filed: Mar. 7, 2018
(86) PCT No.: PCT/US2018/021249

§ 371 Date: Sep. 4, 2019

Related U.S. Application Data
(60) Provisional application No. 62/468,887, filed on Mar. 8, 2017.
(60) Provisional application No. 62/469,366, filed on Mar. 9, 2017.

(51) Int. Cl. C12N 5/0783; C07K 14/725; C07K 14/73; C07K 14/705; C07K 14/715

(52) U.S. Cl. CPC C12N 5/0636; C07K 14/7051; C07K 14/70514; C07K 14/70517; C07K 14/70521; C12N 2501/515; C07K 14/7155; C12N 2501/15; C12N 2501/2302; C12N 2501/2312; C07K 14/70542

(57) ABSTRACT

Disclosed herein are immunostimulatory cells recombinantly engineered for adoptive cellular therapy. Additionally provided are pharmaceutical compositions comprising such immunostimulatory cells and methods of using such immunostimulatory cells to treat cancer or pathogen infections in a subject in need thereof.

### 1. CROSS-REFERENCE TO RELATED APPLICATIONS

This application claims the benefit of U.S. Provisional application No. 62/468,887, filed Mar. 8, 2017, and U.S. Provisional application No. 62/469,366, filed Mar. 9, 2017, each of which is incorporated by reference herein in its entirety.

### 2. REFERENCE TO SEQUENCE LISTING SUBMITTED ELECTRONICALLY

This application incorporates by reference a Sequence Listing with this application as an ASCII text file entitled “13542-044-228_SL.TXT” created on Mar. 2, 2018, and having a size of 82,681 bytes.

### 3. FIELD

The present invention relates generally to cancer treatment and pathogen infection treatment, and more specifically to immunotherapy for cancer treatment and pathogen infection treatment.

### 4. BACKGROUND

Recent years have provided tremendous advancements in the treatment of cancer and pathogen infections (e.g., viral infections). Among these advancements are the use of immunotherapy, where a patient's immune response is harnessed to treat cancer or infection. Such immunotherapy treatment methods include the use of cell-based immunotherapy, where cells of the immune system are utilized for therapeutic treatment. Immune system cells such as T cells and other immune cells can be modified to target tumor antigens.

In response to immune attack, solid tumors upregulate PD-L1 in response to immune attack, which in turn binds PD-1 receptor expressed on T cells, resulting in T-cell inhibition (see Pardoll, Nat. Rev. Cancer 12(4):252-64 (2012)). Upregulation of PD-L1 on T cells and antigen presenting cells (APCs) was described as well, resulting in inhibition of activated T cells (Talay et al., Proc. Natl. Acad. Sci. USA 106(8):2741-2746 (2009); Latchman et al., Proc. Natl. Acad. Sci. USA 101(29):10691-10696 (2004); Liu et al., J. Cell. Mol. Med. 19(6):1223-1233 (2015)). PD-1/PD-L1 checkpoint blockade therapy counteracts this inhibition, thereby leading to activated T cells. Various strategies to inhibit the immune checkpoint blockade mediated by PD-1 have been described, including the use of PD-1 or PD-L1 antibodies (Burga et al., Cancer Immunol. Immunother. 64(7):817-829 (2015); Moon et al., Clin. Cancer Res. 20(16):4262-4273 (2014); John et al., Clin. Cancer Res. 19(20):5636-5646 (2013)), RNA interference (Borkner et al., Cancer Immunol. Immunother. 59(8):1173-1183 (2010)), and co-stimulatory molecules (Prosser et al., Mol. Immunol. 51(3-4):263-272 (2012); Ankri et al., J. Immunol. 191(8):4121-4129 (2013)).

Similarly, functional impairment of T cells is characteristic of many human pathogenic infections, such as viral infections (see Day et al., Nature 443:350-354 (2006) and references cited therein). PD-1 is a negative regulator of activated T cells, and is markedly upregulated on the surface of exhausted virus-specific CD8+ T cells (Ishida et al., EMBO J. 11:3887-3895 (1992); Noshimura et al., Immunity 11:141-151 (1999); Sharpe et al., Nat. Rev. Immunol. 2:116-126 (2002); Che. Nat. Rev. Immunol. 4:336-347 (2004); Barber et al., Nature 439:682-687 (2006)). Blockade of this pathway using antibodies against the PD ligand 1 (PD-L1, also known as CD274) restores CD8+ T-cell function and reduces viral load (Barber et al., Nature 439:682-687 (2006)). It was found that PD-1 is significantly upregulated on T cells, and expression correlates with impaired HIV-specific CD8+ T-cell function as well as predictors of disease progression: positively with plasma viral load and inversely with CD4+ T-cell count (Day et al., Nature 443:350-354 (2006)). PD-1 expression on CD4+ T cells likewise showed a positive correlation with viral load and an inverse correlation with CD4+ T-cell count, and blockade of the pathway augmented HIV-specific CD4+ and CD8+ T-cell function (Day et al., Nature 443:350-354 (2006)). The results described by Day et al. (supra, 2006) indicate that the immunoregulatory PD-1/PD-L1 pathway is operative during a persistent viral infection in humans, and define a reversible defect in HIV-specific T-cell function (Day et al., Nature 443:350-354 (2006)).

While immunotherapy methods have provided new modalities for cancer and infection treatment, including antibody therapies and cell-based therapies using immune cells such as T cells, limitations have been found for the effectiveness of such treatments. For example, malignant cells and infected cells can adapt to generate an immunosuppressive microenvironment that protects the cells from immune recognition and elimination. This microenvironment poses a challenge to methods of treatment involving stimulation of an immune response, including immunotherapy methods such as targeted T cell therapies. Furthermore, solid tumors or infections can be restricted within anatomical compartments such that access of therapeutic immune cells to the tumors or infected cells is limited. In addition, an immunosuppressive microenvironment must be overcome so that the immunotherapy is effective. The successful elimination of cancer cells and the successful elimination of infected cells thus both require overcoming tumor-induced or infection-induced immunosuppression.

Thus, there exists a need for therapies to provide improved treatment of cancer and pathogen infections that overcome microenvironments associated with malignant cells or infected cells that inhibit effective immunotherapies. The present invention satisfies this need and provides related advantages as well.

### 5. SUMMARY OF INVENTION

The invention can be summarized by the claims appended hereto and as described below.

In one aspect, provided herein is a T cell comprising in one or more transgenes: (a) a first nucleotide sequence encoding a dominant negative form of an inhibitor of a cell-mediated immune response of the T cell, and (b) a second nucleotide sequence encoding an immunomodulatory agent, wherein the immunomodulatory agent is a single chain variable fragment (scFv) or peptide antibody, which immunomodulatory agent binds to and inhibits an immune checkpoint inhibitor, and wherein the immune checkpoint inhibitor is different from the inhibitor of a cell-mediated immune response. In certain embodiments, the dominant negative form of the inhibitor of a cell-mediated immune response is expressed as a membrane protein on the T cell surface. In certain embodiments, the inhibitor of a cell-mediated immune response is an immune checkpoint inhibitor.

In certain embodiments of a T cell, the inhibitor of a cell-mediated immune response is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a particular embodiment, the inhibitor of a cell-mediated immune response is PD-1.

In certain embodiments of a T cell, the inhibitor of a cell-mediated immune response is TGF-β receptor.

In certain embodiments of a T cell, the immunomodulatory agent is secreted from the T cell. In certain embodiments of a T cell comprising in one or more transgenes, the immunomodulatory agent is a scFv. In certain embodiments of a T cell comprising in one or more transgenes, the immunomodulatory agent is a peptide antibody.

In certain embodiments of a T cell, the immune checkpoint inhibitor to which the immunomodulatory agent binds is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a particular embodiment, the immune checkpoint inhibitor to which the immunomodulatory agent binds is TIM-3. In another particular embodiment, the immune checkpoint inhibitor to which the immunomodulatory agent binds is LAG-3.

In certain embodiments of a T cell, the T cell comprises a transgene comprising (a) the first nucleotide sequence encoding a dominant negative form of an inhibitor of a cell-mediated immune response of the T cell, and (b) the second nucleotide sequence encoding an immunomodulatory agent, wherein a nucleotide sequence encoding a cleavable linker is present in between the first nucleotide sequence encoding a dominant negative form and the second nucleotide sequence encoding an immunomodulatory agent, and wherein expression of the transgene is under control of a promoter such that the transgene is expressible in the T cell to produce the dominant negative form and the immunomodulatory agent. In certain embodiments, the promoter is constitutive. In certain embodiments, the transgene further comprises a third nucleotide sequence encoding a reporter, wherein a nucleotide sequence encoding a cleavable linker is present in between any adjacent occurrences in the transgene of the first nucleotide sequence encoding a dominant negative form of an inhibitor of a cell-mediated immune response of the T cell, the second nucleotide sequence encoding an immunomodulatory agent, and the third nucleotide sequence encoding a reporter, and wherein the transgene is expressible in the T cell to produce the reporter.

In certain embodiments of a T cell, the T cell recognizes and is sensitized to a target antigen associated with a mammalian disease or disorder.

In certain embodiments of a T cell, the T cell further comprises a fourth nucleotide sequence encoding a chimeric antigen receptor (CAR), wherein the CAR binds to a target antigen that is associated with a mammalian disease or disorder.

In certain embodiments of a T cell, the transgene further comprises a fourth nucleotide sequence encoding a chimeric antigen receptor (CAR), wherein the CAR binds to a target antigen that is associated with a mammalian disease or disorder, and wherein a nucleotide sequence encoding a cleavable linker is present in between any adjacent occurrences in the transgene of the first nucleotide sequence encoding a dominant negative form of an inhibitor of a cell-mediated immune response of the T cell, the second nucleotide sequence encoding an immunomodulatory agent, and the fourth nucleotide sequence encoding a CAR, and wherein the transgene is expressible in the T cell to produce the CAR.

In certain embodiments of a T cell, the transgene further comprises a fourth nucleotide sequence encoding a chimeric antigen receptor (CAR), wherein the CAR binds to a target antigen that is associated with a mammalian disease or disorder, and wherein a nucleotide sequence encoding a cleavable linker is present in between any adjacent occurrences in the transgene of the first nucleotide sequence encoding a dominant negative form of an inhibitor of a cell-mediated immune response of the T cell, the second nucleotide sequence encoding an immunomodulatory agent, the third nucleotide sequence encoding a reporter, and the fourth nucleotide sequence encoding a CAR, and wherein the transgene is expressible in the T cell to produce the CAR.

In another aspect, provided herein is a T cell comprising a transgene, which transgene comprises a first nucleotide sequence encoding a dominant negative form of an inhibitor of a cell-mediated immune response of the T cell, wherein expression of the transgene is under control of an inducible promoter, which inducible promoter is induced upon activation of the T cell. In certain embodiments, the inducible promoter is induced by nuclear factor of activated T cells (NFAT) binding. In certain embodiments, the dominant negative form of the inhibitor of a cell-mediated immune response is expressed as a membrane protein on the T cell surface.

In certain embodiments of a T cell comprising a transgene, the inhibitor of a cell-mediated immune response is an immune checkpoint inhibitor.

In certain embodiments, the inhibitor of a cell-mediated immune response is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a particular embodiment, the inhibitor of a cell-mediated immune response is PD-1.

In certain embodiments, the inhibitor of a cell-mediated immune response is TGF-β receptor.

In certain embodiments of a T cell comprising a transgene, the transgene further comprises a second nucleotide sequence encoding a reporter, wherein a nucleotide sequence encoding a cleavable linker is present in between the first nucleotide sequence encoding a dominant negative form of an inhibitor of a cell-mediated immune response of the T cell and the second nucleotide sequence encoding a reporter, and wherein the transgene is expressible in the T cell to produce the reporter.

In certain embodiments of a T cell comprising a transgene, the T cell recognizes and is sensitized to a target antigen associated with a mammalian disease or disorder.

In another aspect, provided herein is a T cell comprising a transgene, which transgene comprises a first nucleotide sequence encoding an immunomodulatory agent, wherein expression of the transgene is under control of an inducible promoter, which inducible promoter is induced upon activation of the T cell, wherein the immunomodulatory agent is a single chain variable fragment (scFv) or peptide antibody, which immunomodulatory agent binds to and inhibits an immune checkpoint inhibitor. In certain embodiments, the inducible promoter is induced by nuclear factor of activated T cells (NFAT) binding.

In certain embodiments of a T cell comprising a transgene, the immunomodulatory agent is secreted from the T cell.

In certain embodiments, the immunomodulatory agent is a scFv. In certain embodiments, the immunomodulatory agent is a peptide antibody.

In certain embodiments of a T cell comprising a transgene, the immune checkpoint inhibitor to which the immunomodulatory agent binds is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a particular embodiment, the immune checkpoint inhibitor to which the immunomodulatory agent binds is TIM-3. In another particular embodiment, the immune checkpoint inhibitor to which the immunomodulatory agent binds is LAG-3.

In certain embodiments of a T cell comprising a transgene, the transgene further comprises a second nucleotide sequence encoding a reporter, wherein a nucleotide sequence encoding a cleavable linker is present in between the first nucleotide sequence encoding an immunomodulatory agent and the second nucleotide sequence encoding a reporter, and wherein the transgene is expressible in the T cell to produce the reporter.

In certain embodiments of a T cell comprising a transgene, the T cell recognizes and is sensitized to a target antigen associated with a mammalian disease or disorder.

In certain embodiments, the mammalian disease or disorder is a cancer and the target antigen is a cancer antigen. In certain embodiments, the cancer antigen is selected from the group consisting of mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (LlCAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF- R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Rα, Latent Membrane Protein 1 (LMP1), Neural cell-adhesion molecule (N-CAM/CD56), and Trail Receptor (TRAIL R). In a particular embodiment, the cancer antigen is mesothelin.

In certain embodiments, the mammalian disease or disorder is an infection with a pathogen and the target antigen is an antigen of the pathogen. In certain embodiments, the pathogen is a human pathogen. In certain embodiments, the pathogen is a virus, a bacterium, a fungus, a protozoan, a helminth, or a protist.

In certain embodiments, the target antigen is a viral antigen. In certain embodiments, the viral antigen can elicit an immune response in a human subject infected with the virus.

In certain embodiments, the viral antigen is selected from the group consisting of a human immunodeficiency virus (HIV) antigen, a hepatitis B virus (HBV) antigen, a hepatitis C virus (HCV) antigen, a herpes simplex virus (HSV) antigen, a varicella zoster virus (VZV) antigen, an adenovirus antigen, a cytomegalovirus (CMV) antigen, and an Epstein-Barr virus (EBV) antigen.

In certain embodiments, the viral antigen is a HIV antigen selected from the group consisting of group-specific antigen (gag) protein, p55, p24, p18, envelope glycoprotein (env), gp160, gp120, gp41, reverse transcriptase (pol), p66, and p31.

In certain embodiments, the viral antigen is a HBV antigen selected from the group consisting of HBV envelope protein S, HBV envelope protein M, HBV envelope protein L, and the S domain of HBV envelope protein S, M or L.

In certain embodiments, the viral antigen is a HCV antigen selected from the group consisting of core protein, envelope protein E1, envelope protein E2, NS2, NS3, NS4, and NS5.

In certain embodiments, the viral antigen is a HSV antigen selected from the group consisting of gE, gI, gB, gD, gH, gL, gC, gG, gK, gM, and the extracellular domain of gE.

In certain embodiments, the viral antigen is a VZV antigen selected from the group consisting of gE and gl.

In certain embodiments, the viral antigen is an adenovirus antigen selected from the group consisting of hexon protein and penton protein.

In certain embodiments, the viral antigen is a CMV antigen selected from the group consisting of pp65, immediate early (IE) antigen, and IEl.

In certain embodiments, the viral antigen is an EBV antigen selected from the group consisting of latent membrane protein 2 (LMP2), Epstein-Barr nuclear antigen 1 (EBNA1), and BZLF1.

In certain embodiments of a T cell, the T cell further recombinantly expresses a suicide gene. In certain embodiments, the suicide gene comprises inducible Caspase 9.

In certain embodiments of a T cell, the T cell is a cytotoxic T lymphocyte (CTL). In certain embodiments, the T cell is CD4+. In certain embodiments, the T cell is CD8+.

In certain embodiments of a T cell, the T cell is derived from a human.

In another aspect, provided herein is an immunostimulatory cell comprising in one or more transgenes: (a) a first nucleotide sequence encoding a chimeric antigen receptor (CAR), wherein the CAR binds to a target antigen associated with a mammalian disease or disorder, (b) a second nucleotide sequence encoding a dominant negative form of an inhibitor of a cell-mediated immune response of the immunostimulatory cell, and (c) a third a nucleotide sequence encoding a membrane bound form of interleukin 12 (membrane IL-12).

In certain embodiments, the immunostimulatory cell comprises a transgene comprising: (a) the first nucleotide sequence encoding a chimeric antigen receptor (CAR), (b) the second nucleotide sequence encoding a dominant negative form of an inhibitor of a cell-mediated immune response of the immunostimulatory cell, and (c) the third nucleotide sequence encoding a membrane bound form of interleukin 12 (membrane IL-12), wherein a nucleotide sequence encoding a cleavable linker is present in between any adjacent occurrences in the transgene of the first nucleotide sequence encoding a CAR, the second nucleotide sequence encoding a dominant negative form of an inhibitor of a cell-mediated immune response of the immunostimulatory cell, and the third nucleotide sequence encoding a membrane IL-12, and wherein expression of the transgene is under control of a promoter such that the transgene is expressible in the immunostimulatory cell to produce the CAR, the dominant negative form and the membrane IL-12. In certain embodiments, the promoter is constitutive.

In certain embodiments, the immunostimulatory cell comprises: (1) a first transgene, which first transgene comprises: (a) the first nucleotide sequence encoding a chimeric antigen receptor (CAR), and (b) the second nucleotide sequence encoding a dominant negative form of an inhibitor of a cell-mediated immune response of the immunostimulatory cell, and (2) a second transgene, which second transgene comprises (c) the third nucleotide sequence encoding a membrane bound form of interleukin 12 (membrane IL-12), wherein a nucleotide sequence encoding a cleavable linker is present in between the first nucleotide sequence encoding a CAR and the second nucleotide sequence encoding a dominant negative form of an inhibitor of a cell-mediated immune response of the immunostimulatory cell, and wherein expression of the first transgene is under control of a promoter such that the first transgene is expressible in the immunostimulatory cell to produce the CAR and the dominant negative form, and wherein expression of the second transgene is under control of an inducible promoter, which inducible promoter is induced upon activation of the immunostimulatory cell. In certain embodiments, the promoter is constitutive.

In certain embodiments, the immunostimulatory cell comprises: (1) a first transgene, which first transgene comprises (a) the nucleotide sequence encoding a chimeric antigen receptor (CAR), (2) a second transgene, which second transgene comprises (b) the nucleotide sequence encoding a dominant negative form of an inhibitor of a cell-mediated immune response of the immunostimulatory cell, and (3) a third transgene, which third transgene comprises (c) the nucleotide sequence encoding a membrane bound form of interleukin 12 (membrane IL-12), wherein expression of the third transgene is under control of an inducible promoter, which inducible promoter is induced upon activation of the immunostimulatory cell.

In certain embodiments of an immunostimulatory cell, the first and the second transgenes are under control of a constitutive promoter.

In certain embodiments of an immunostimulatory cell, the inducible promoter is induced by nuclear factor of activated T cells (NFAT) binding.

In another aspect, provided herein is an immunostimulatory cell comprising: (1) in one or more transgenes: (a) a first nucleotide sequence encoding a chimeric antigen receptor (CAR), wherein the CAR binds to a target antigen associated with a mammalian disease or disorder, (b) a second nucleotide sequence encoding a dominant negative form of an inhibitor of a cell-mediated immune response of the immunostimulatory cell, that is a receptor-synthetic Notch fusion protein comprising (i) an extracellular domain of the inhibitor of a cell-mediated immune response of the immunostimulatory cell, (ii) the transmembrane core domain of Notch C-terminal to the extracellular domain, and (iii) a transcription factor C-terminal to the transmembrane core domain of Notch, and (2) in a different transgene (c) a third nucleotide sequence encoding a membrane bound form of interleukin 12 (membrane IL-12), wherein expression of the membrane IL-12 is under control of an inducible promoter, which inducible promoter is induced upon binding of the transcription factor, and wherein the transcription factor is cleaved from the receptor-synthetic Notch fusion protein intracellularly upon binding of the extracellular domain to its ligand.

In certain embodiments, the immunostimulatory cell comprises: (1) a first transgene, which first transgene comprises: (a) the first nucleotide sequence encoding a chimeric antigen receptor (CAR), and (b) the second nucleotide sequence encoding a receptor-synthetic Notch fusion protein, and (2) a second transgene, which second transgene comprises (c) the third nucleotide sequence encoding a membrane IL-12, wherein a nucleotide sequence encoding a cleavable linker is present in between the first nucleotide sequence encoding a CAR and the second nucleotide sequence encoding a receptor-synthetic Notch fusion protein, and wherein expression of the first transgene is under control of a promoter such that the first transgene is expressible in the immunostimulatory cell to produce the CAR and the receptor-synthetic Notch fusion protein, and wherein expression of the second transgene is under control of an inducible promoter, which inducible promoter is induced upon binding of the transcription factor, and wherein the transcription factor is cleaved from the receptor-synthetic Notch fusion protein intracellularly upon binding of the extracellular domain to its ligand. In certain embodiments, the promoter is constitutive.

In certain embodiments, the immunostimulatory cell comprises: (1) a first transgene, which first transgene comprises: (a) the first nucleotide sequence encoding a chimeric antigen receptor (CAR), (2) a second transgene, which second transgene comprises: (b) the second nucleotide sequence encoding a receptor-synthetic Notch fusion protein, and (3) a third transgene, which third transgene comprises (c) the third nucleotide sequence encoding a membrane IL-12, wherein the first and second transgenes are expressible in the immunostimulatory cell to produce the CAR and the receptor-synthetic Notch fusion protein, and wherein expression of the second transgene is under control of an inducible promoter, which inducible promoter is induced upon binding of the transcription factor, and wherein the transcription factor is cleaved from the receptor-synthetic Notch fusion protein intracellularly upon binding of the extracellular domain to its ligand. In certain embodiments, the first and second transgenes are under control of constitutive promoters.

In certain embodiments of an immunostimulatory cell, the membrane IL-12 comprises a p40 subunit and a p35 subunit separated by a linker, and wherein the p35 subunit is fused to a transmembrane domain.

In another aspect, provided herein is an immunostimulatory cell comprising in one or more transgenes: (a) a first nucleotide sequence encoding a dominant negative form of an inhibitor of a cell-mediated immune response of the immunostimulatory cell, and (b) a second nucleotide sequence encoding interleukin 12 (IL-12), wherein the IL-12 when expressed by the immunostimulatory cell is secreted from the immunostimulatory cell.

In certain embodiments, the immunostimulatory cell comprises a transgene comprising: (a) the first nucleotide sequence encoding a dominant negative form of an inhibitor of a cell-mediated immune response of the immunostimulatory cell, and (b) the second nucleotide sequence encoding interleukin 12 (IL-12), wherein the first nucleotide sequence encoding the dominant negative form and the second nucleotide sequence encoding the IL-12 are separated by an internal ribosome entry site (IRES), wherein expression of the transgene is under control of a promoter such that the transgene is expressible in the immunostimulatory cell to produce the dominant negative form and the IL-12. In certain embodiments, the promoter is constitutive.

In certain embodiments, the immunostimulatory cell comprises: (1) a first transgene comprising (a) the first nucleotide sequence encoding a dominant negative form of an inhibitor of a cell-mediated immune response of the immunostimulatory cell, and (2) a second transgene comprising (b) the second nucleotide sequence encoding interleukin 12 (IL-12), wherein expression of the dominant negative form is under control of a promoter such that the first transgene is expressible in the immunostimulatory cell to produce the dominant negative form, wherein expression of the IL-12 is under control of an inducible promoter, which inducible promoter is induced upon activation of the immunostimulatory cell, and wherein the IL-12 when expressed by the immunostimulatory cell is secreted from the immunostimulatory cell. In certain embodiments, the promoter is constitutive.

In certain embodiments of an immunostimulatory cell, the inducible promoter is induced by nuclear factor of activated T cells (NFAT) binding.

In certain embodiments, the immunostimulatory cell recognizes and is sensitized to a target antigen associated with a mammalian disease or disorder.

In certain embodiments of an immunoinhibitory cell, the mammalian disease or disorder is a cancer and the target antigen is a cancer antigen.

In certain embodiments, the cancer antigen is selected from the group consisting of mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (LlCAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF- R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Ra, Latent Membrane Protein 1 (LMP1), Neural cell-adhesion molecule (N-CAM/CD56), and Trail Receptor (TRAIL R). In a particular embodiment, the cancer antigen is mesothelin.

In certain embodiments of an immunostimulatory cell, the mammalian disease or disorder is an infection with a pathogen and the target antigen is an antigen of the pathogen. In certain embodiments, the pathogen is a human pathogen. In certain embodiments, the pathogen is a virus, a bacterium, a fungus, a protozoan, a helminth, or a protist.

In certain embodiments of an immunostimulatory cell, the target antigen is a viral antigen. In certain embodiments, the viral antigen can elicit an immune response in a human subject infected with the virus.

In certain embodiments of an immunostimulatory cell, the viral antigen is selected from the group consisting of a human immunodeficiency virus (HIV) antigen, a hepatitis B virus (HBV) antigen, a hepatitis C virus (HCV) antigen, a herpes simplex virus (HSV) antigen, a varicella zoster virus (VZV) antigen, an adenovirus antigen, a cytomegalovirus (CMV) antigen, and an Epstein-Barr virus (EBV) antigen.

In certain embodiments of an immunostimulatory cell, the viral antigen is a HIV antigen selected from the group consisting of group-specific antigen (gag) protein, p55, p24, p18, envelope glycoprotein (env), gp160, gp120, gp41, reverse transcriptase (pol), p66, and p31.

In certain embodiments of an immunostimulatory cell, the viral antigen is a HBV antigen selected from the group consisting of HBV envelope protein S, HBV envelope protein M, HBV envelope protein L, and the S domain of HBV envelope protein S, M or L.

In certain embodiments of an immunostimulatory cell, the viral antigen is a HCV antigen selected from the group consisting of core protein, envelope protein E1, envelope protein E2, NS2, NS3, NS4, and NS5.

In certain embodiments of an immunostimulatory cell, the viral antigen is a HSV antigen selected from the group consisting of gE, gI, gB, gD, gH, gL, gC, gG, gK, gM, and the extracellular domain of gE.

In certain embodiments of an immunostimulatory cell, the viral antigen is a VZV antigen selected from the group consisting of gE and gl.

In certain embodiments of an immunostimulatory cell, the viral antigen is an adenovirus antigen selected from the group consisting of hexon protein and penton protein.

In certain embodiments of an immunostimulatory cell, the viral antigen is a CMV antigen selected from the group consisting of pp65, immediate early (IE) antigen, and IE1.

In certain embodiments of an immunostimulatory cell, the viral antigen is an EBV antigen selected from the group consisting of latent membrane protein 2 (LMP2), Epstein-Barr nuclear antigen 1 (EBNA1), and BZLF1.

In certain embodiments of an immunostimulatory cell, the dominant negative form of the inhibitor of a cell-mediated immune response is expressed as a membrane protein on the immunostimulatory cell surface. In certain embodiments, the inhibitor of a cell-mediated immune response is an immune checkpoint inhibitor.

In certain embodiments of an immunostimulatory cell, the inhibitor of a cell-mediated immune response is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a particular embodiment, the inhibitor of a cell-mediated immune response is PD-1.

In certain embodiments of an immunostimulatory cell, he inhibitor of a cell-mediated immune response is TGF-β receptor.

In certain embodiments, the immunostimulatory cell is a T cell. In certain embodiments, the T cell is a cytotoxic T lymphocyte (CTL). In certain embodiments, the T cell is CD4+. In certain embodiments, the T cell is CD8+.

In certain embodiments, the immunostimulatory cell is a Natural Killer (NK) cell.

In certain embodiments, the immunostimulatory cell further recombinantly expresses a suicide gene. In certain embodiments, the suicide gene comprises inducible Caspase 9.

In certain embodiments, the immunostimulatory cell is derived from a human.

In another aspect, provided herein is a pharmaceutical composition comprising a therapeutically effective amount of a T cell or an immunostimulatory cell as described above.

In another aspect, provided herein is a method of treating a cancer in a subject in need thereof, comprising administering to the subject a therapeutically effective amount of a T cell or an immunostimulatory cell as described above.

In another aspect, provided herein is a method of treating an infection with a pathogen in a subject in need thereof, comprising administering to the subject a therapeutically effective amount of a T cell or an immunostimulatory cell as described above.

In another aspect, provided herein is a method of treating a cancer in a subject in need thereof, comprising administering to the subject pharmaceutical composition comprising a T cell or an immunostimulatory cell as described above.

In another aspect, provided herein is a method of treating an infection with a pathogen in a subject in need thereof, comprising administering to the subject a pharmaceutical composition comprising a T cell or an immunostimulatory cell as described above.

In certain embodiments of the methods, the cancer is selected from the group consisting of mesothelioma, lung cancer, pancreatic cancer, ovarian cancer, breast cancer, colon cancer, pleural tumor, glioblastoma, esophageal cancer, gastric cancer, and synovial sarcoma.

In certain embodiments of the methods, the infection with a pathogen is an infection with a virus, a bacterium, a fungus, a protozoan, a helminth, or a protist. In a particular embodiment, the infection with a pathogen is an infection with a virus.

In certain embodiments of the methods, the infection with a pathogen is an infection with HCV, HIV, HBV, HSV, VZV, adenovirus, CMV or EBV.

In certain embodiments of the methods, the subject is a human.

In certain embodiments of the methods, the administering is by intrapleural administration, intravenous administration, subcutaneous administration, intranodal administration, intratumoral administration, intrathecal administration, intraperitoneal administration, intracranial administration, or direct administration to the thymus.

In certain embodiments of the methods, the T cell or the immunostimulatory cell is administered in a dose in the range of 10⁴ to 10¹⁰ cells per kilogram of body weight. In certain embodiments, the dose is in the range of 3×10⁵ to 3×10⁶ cells per kilogram of body weight.

### 6. DESCRIPTION OF THE DRAWINGS

FIG. 1. Illustration of a construct that contains a nucleotide sequence encoding a mesothelin (MSLN)-specific chimeric antigen receptor (CAR), a nucleotide sequence encoding a dominant negative form of PD-1, a nucleotide sequence encoding a single chain variable fragment (scFv) that binds to TIM-3, and a nucleotide sequence encoding a LNGFR reporter. The adjacent nucleotide sequences encoding different proteins are separated by a nucleotide sequence encoding a 2A peptide. The CAR contains a CD3ζ endodomain, a CD28 transmembrane (TM) domain, and a CD28 cytoplasmic (CYT) domain (as the costimulatory domain). LTR, long terminal repeat; LS, leader sequence; SA, splice acceptor; SD, splice donor.

FIG. 2. Illustration of a construct that contains a nucleotide sequence encoding a mesothelin-specific CAR, a nucleotide sequence encoding a dominant negative form of PD-1, a nucleotide sequence encoding an scFv that binds to LAG-3, and a nucleotide sequence encoding a LNGFR reporter. The adjacent nucleotide sequences encoding different proteins are separated by a nucleotide sequence encoding a 2A peptide. The CAR contains a CD3ζ endodomain, a CD28 transmembrane domain, and a CD28 cytoplasmic domain (as the costimulatory domain). LTR, long terminal repeat; LS, leader sequence; SA, splice acceptor; SD, splice donor.

FIG. 3. Illustration of a construct that contains a nucleotide sequence encoding a mesothelin-specific CAR, a nucleotide sequence encoding a dominant negative form of PD-1, a nucleotide sequence encoding an scFv that binds to TIM-3, and a nucleotide sequence encoding a LNGFR reporter. The adjacent nucleotide sequences encoding different proteins are separated by a nucleotide sequence encoding a 2A peptide. The CAR contains a CD3ζ endodomain, a CD28 transmembrane domain, and a 4-1BB cytoplasmic domain (as the costimulatory domain). LTR, long terminal repeat; LS, leader sequence; SA, splice acceptor; SD, splice donor.

FIG. 4. Illustration of a construct that contains a nucleotide sequence encoding a mesothelin-specific CAR, a nucleotide sequence encoding a dominant negative form of PD-1, a nucleotide sequence encoding an scFv that binds to LAG-3, and a nucleotide sequence encoding a LNGFR reporter. The adjacent nucleotide sequences encoding different proteins are separated by a nucleotide sequence encoding a 2A peptide. The CAR contains a CD3ζ endodomain, a CD28 transmembrane domain, and a 4-1BB cytoplasmic domain (as the costimulatory domain). LTR, long terminal repeat; LS, leader sequence; SA, splice acceptor; SD, splice donor.

FIG. 5. Illustration of a construct that contains a nucleotide sequence encoding a dominant negative form of PD-1, a nucleotide sequence encoding an scFv that binds to TIM-3, and a nucleotide sequence encoding an mCherry reporter. The adjacent nucleotide sequences encoding different proteins are separated by a nucleotide sequence encoding a 2A peptide. LTR, long terminal repeat; LS, leader sequence; SA, splice acceptor; SD, splice donor.

FIG. 6. Illustration of a construct that contains a nucleotide sequence encoding a dominant negative form of PD-1, a nucleotide sequence encoding an scFv that binds to LAG-3, and a nucleotide sequence encoding an mCherry reporter. The adjacent nucleotide sequences encoding different proteins are separated by a nucleotide sequence encoding a 2A peptide. LTR, long terminal repeat; LS, leader sequence; SA, splice acceptor; SD, splice donor.

FIG. 7. Illustration of a construct that contains a nucleotide sequence encoding a dominant negative form of PD-1, whose expression is under control of an inducible promoter containing NFAT responsive elements, and a nucleotide sequence encoding an mCherry reporter. The two nucleotide sequences are separated by a nucleotide sequence encoding a 2A peptide. LTR, long terminal repeat; LS, leader sequence; SA, splice acceptor; SD, splice donor.

FIG. 8. Illustration of a construct that contains a nucleotide sequence encoding an scFv that binds to TIM-3, whose expression is under control of an inducible promoter containing NFAT responsive elements, and a nucleotide sequence encoding an mCherry reporter. The two nucleotide sequences are separated by a nucleotide sequence encoding a 2A peptide. LTR, long terminal repeat; LS, leader sequence; SA, splice acceptor; SD, splice donor.

FIG. 9. Illustration of a construct that contains a nucleotide sequence encoding an scFv that binds to LAG-3, whose expression is under control of an inducible promoter containing NFAT responsive elements, and a nucleotide sequence encoding an mCherry reporter. The two nucleotide sequences are separated by a nucleotide sequence encoding a 2A peptide. LTR, long terminal repeat; LS, leader sequence; SA, splice acceptor; SD, splice donor.

FIG. 10. Illustration of a construct that contains a nucleotide sequence encoding a mesothelin-specific CAR, a nucleotide sequence encoding a dominant negative form of PD-1, and a nucleotide sequence encoding a membrane IL-12. The membrane IL-12 contains p40 and p35 subunits separated by a linker, and the p35 subunit is fused to the transmembrane domain of CD8. The adjacent nucleotide sequences encoding different proteins are separated by a nucleotide sequence encoding a P2A peptide. The CAR contains a CD3ζ endodomain, a CD28 transmembrane domain, and a CD28 or 4-1BB cytoplasmic domain (as the costimulatory domain).

FIG. 11. Illustration of a first construct that contains a nucleotide sequence encoding a mesothelin-specific CAR, and a nucleotide sequence encoding a dominant negative form of PD-1, and a second construct containing a nucleotide sequence encoding a membrane IL-12, whose expression is under control of an inducible promoter containing NFAT responsive elements. The membrane IL-12 contains p40 and p35 subunits separated by a linker, and the p35 subunit is fused to the transmembrane domain of CD8. The two nucleotide sequences on the first construct are separated by a nucleotide sequence encoding a P2A peptide. The CAR contains a CD3ζ endodomain, a CD28 transmembrane domain, and a CD28 or 4-1BB cytoplasmic domain (as the costimulatory domain).

FIG. 12A-FIG. 12B. FIG. 12A Illustration of a first construct that contains a nucleotide sequence encoding a mesothelin-specific CAR, and a nucleotide sequence encoding a receptor-synthetic Notch fusion protein (which contains an extracellular domain of PD-1, the transmembrane core domain of Notch C-terminal to the extracellular domain, and a transcription factor C-terminal to the transmembrane core domain of Notch), and a second construct containing a nucleotide sequence encoding a membrane IL-12, whose expression is under control of an inducible promoter containing responsive elements of the transcription factor. The membrane IL-12 contains p40 and p35 subunits separated by a linker, and the p35 subunit is fused to the transmembrane domain of CD8. The two nucleotide sequences on the first construct are separated by a nucleotide sequence encoding a P2A peptide. The CAR contains a CD3ζ endodomain, a CD28 transmembrane domain, and a CD28 or 4-1BB cytoplasmic domain (as the costimulatory domain). FIG. 12B Scheme illustrating expression of membrane IL-12 induced by the transcription factor released from the receptor-synthetic Notch fusion protein.

FIG. 13. Illustration of a construct that contains a nucleotide sequence encoding a dominant negative form of PD-1, an internal ribosome entry site (IRES), and a nucleotide sequence encoding soluble IL-12. LTR, long terminal repeat.

FIG. 14. Illustration of a construct that contains a nucleotide sequence encoding a dominant negative form of PD-1, and a nucleotide sequence encoding soluble IL-12. The expression of soluble IL-12 is under control of an inducible promoter containing NFAT responsive elements. LTR, long terminal repeat.

FIG. 15. Illustration of a construct that contains a nucleotide sequence encoding a mesothelin-specific CAR, a nucleotide sequence encoding a dominant negative form of TGF-β receptor, a nucleotide sequence encoding an scFv that binds to TIM-3, and a nucleotide sequence encoding a LNGFR reporter. The adjacent nucleotide sequences encoding different proteins are separated by a nucleotide sequence encoding a 2A peptide. The CAR contains a CD3ζ endodomain, a CD28 transmembrane domain, and a CD28 cytoplasmic domain (as the costimulatory domain). LTR, long terminal repeat; LS, leader sequence; SA, splice acceptor; SD, splice donor.

FIG. 16. Illustration of a construct that contains a nucleotide sequence encoding a mesothelin-specific CAR, a nucleotide sequence encoding a dominant negative form of TGF-β receptor, a nucleotide sequence encoding an scFv that binds to LAG-3, and a nucleotide sequence encoding a LNGFR reporter. The adjacent nucleotide sequences encoding different proteins are separated by a nucleotide sequence encoding a 2A peptide. The CAR contains a CD3ζ endodomain, a CD28 transmembrane domain, and a CD28 cytoplasmic domain (as the costimulatory domain). LTR, long terminal repeat; LS, leader sequence; SA, splice acceptor; SD, splice donor.

FIG. 17. Illustration of a construct that contains a nucleotide sequence encoding a mesothelin-specific CAR, a nucleotide sequence encoding a dominant negative form of TGF-β receptor, a nucleotide sequence encoding an scFv that binds to TIM-3, and a nucleotide sequence encoding a LNGFR reporter. The adjacent nucleotide sequences encoding different proteins are separated by a nucleotide sequence encoding a 2A peptide. The CAR contains a CD3ζ endodomain, a CD28 transmembrane domain, and a 4-1BB cytoplasmic domain (as the costimulatory domain). LTR, long terminal repeat; LS, leader sequence; SA, splice acceptor; SD, splice donor.

FIG. 18. Illustration of a construct that contains a nucleotide sequence encoding a mesothelin-specific CAR, a nucleotide sequence encoding a dominant negative form of TGF-β receptor, a nucleotide sequence encoding an scFv that binds to LAG-3, and a nucleotide sequence encoding a LNGFR reporter. The adjacent nucleotide sequences encoding different proteins are separated by a nucleotide sequence encoding a 2A peptide. The CAR contains a CD3ζ endodomain, a CD28 transmembrane domain, and a 4-1BB cytoplasmic domain (as the costimulatory domain). LTR, long terminal repeat; LS, leader sequence; SA, splice acceptor; SD, splice donor.

### 7. DETAILED DESCRIPTION OF THE INVENTION

The present invention relates to compositions and methods for treating cancer and pathogen infections (for example, viral infections). It is known that malignant cells and infected cells can adapt to generate an immunosuppressive microenvironment to protect the cells from immune recognition and elimination. The immunosuppressive microenvironment provides a mechanism for cancer cells, tumors, and infected cells to inhibit the effects of a patient's immune system to avoid their elimination. This microenvironment poses a challenge to methods of treatment involving stimulation of an immune response, including immunotherapy methods such as targeted T cell therapies. Although inhibition of certain inhibitors of cell-mediated immune response, such as immune checkpoint inhibitors, has been explored to overcome the immunosuppressive microenvironment, the overcoming effect is usually transient because inhibition of one inhibitor of cell-mediated immune response can result in upregulation of another inhibitor of cell-mediated immune response. According to the present invention, the effectiveness of cell-based immunotherapy methods can be enhanced by modifying the cells used in immunotherapy to express certain combinations of proteins to enhance or prolong the effect of overcoming the immunosuppressive microenvironment. As described herein, immunotherapy cells can be genetically engineered to intrinsically express a dominant negative form of an inhibitor of a cell-mediated immune response and an immunomodulatory agent that inhibits an immune checkpoint inhibitor. In addition, immunotherapy cells can be genetically engineered to intrinsically express a dominant negative form of an inhibitor of a cell-mediated immune response or an immunomodulatory agent that inhibits an immune checkpoint inhibitor, and an interleukin-12 (IL-12) protein (in particular, a membrane-bound IL-12 protein, which has reduced side effects as compared with secreted IL-12). Furthermore, the expression of the dominant negative form of an inhibitor of a cell-mediated immune response, the immunomodulatory agent that inhibits an immune checkpoint inhibitor, and/or the IL-12 protein (for example, a membrane-bound IL-12 protein) can be under the control of an inducible promoter to limit their immunostimulatory effects to activated immune cells. By expressing the combination of proteins, immune cells can provide a more effective immune response against the cancer or the infection. By limiting the immunostimulatory effects to activated immune cells, the side effects associated with immunostimulation can be reduced or avoided.

### 7.1 Cells

In one aspect, the invention provides immunostimulatory cells comprising in one or more transgenes: (a) a nucleotide sequence encoding a dominant negative form of an inhibitor of a cell-mediated immune response of the immunostimulatory cell, and (b) a nucleotide sequence encoding an immunomodulatory agent, wherein the immunomodulatory agent is a single chain variable fragment (scFv) or peptide antibody, which immunomodulatory agent binds to and inhibits an immune checkpoint inhibitor, and wherein the immune checkpoint inhibitor is different from the inhibitor of a cell-mediated immune response.

The nucleotide sequence encoding a dominant negative form and the nucleotide sequence encoding an immunomodulatory agent can be present in two different transgenes or preferably in one single transgene. In specific embodiments, they are present in one single transgene (i.e., the immunostimulatory cells comprise a transgene comprising: the nucleotide sequence encoding a dominant negative form of an inhibitor of a cell-mediated immune response of the immunostimulatory cell, and the nucleotide sequence encoding an immunomodulatory agent, wherein expression of the transgene is under control of a promoter (for example, a constitutive promoter) such that the transgene is expressible in the immunostimulatory cell to produce the dominant negative form and the immunomodulatory agent).

In various embodiments of the aspect, the immunostimulatory cell further comprises a nucleotide sequence encoding a reporter. The nucleotide sequence encoding a reporter, the nucleotide sequence encoding a dominant negative form, and the nucleotide sequence encoding an immunomodulatory agent can be present in two different transgenes, in three different transgenes, or preferably in one single transgene. In specific embodiments, they are present in one single transgene (i.e., the immunostimulatory cell comprises a transgene comprising: the nucleotide sequence encoding the dominant negative form of an inhibitor of a cell-mediated immune response of the immunostimulatory cell, the nucleotide sequence encoding an immunomodulatory agent, and the nucleotide sequence encoding a reporter, wherein the transgene is expressible in the immunostimulatory cell to produce the dominant negative form, the immunomodulatory agent, and the reporter).

In various embodiments of the aspect, the immunostimulatory cell further comprises a nucleotide sequence encoding a chimeric antigen receptor (CAR), wherein the CAR binds to a target antigen associated with a mammalian disease or disorder (i.e., a cancer or an infection with a pathogen). In some embodiments, the immunostimulatory cell further comprises a nucleotide sequence encoding a chimeric antigen receptor (CAR), wherein the CAR binds to a target antigen that is a cancer antigen. In other embodiments, the immunostimulatory cell further comprises a nucleotide sequence encoding a chimeric antigen receptor (CAR), wherein the CAR binds to a target antigen of a pathogen. The nucleotide sequence encoding a CAR, the nucleotide sequence encoding a dominant negative form, and the nucleotide sequence encoding an immunomodulatory agent can be present in two different transgenes, in three different transgenes, or preferably in one single transgene. In specific embodiments, they are present in one single transgene (i.e., the immunostimulatory cell comprises a transgene comprising: the nucleotide sequence encoding the dominant negative form of an inhibitor of a cell-mediated immune response of the immunostimulatory cell, the nucleotide sequence encoding an immunomodulatory agent, and the nucleotide sequence encoding a chimeric antigen receptor (CAR), wherein the transgene is expressible in the immunostimulatory cell to produce the dominant negative form, the immunomodulatory agent, and the CAR). In specific embodiments, the immunostimulatory cell further comprises a nucleotide sequence encoding a reporter. The nucleotide sequence encoding a reporter, the nucleotide sequence encoding a CAR, the nucleotide sequence encoding a dominant negative form, and the nucleotide sequence encoding an immunomodulatory agent can be present in two different transgenes, in three different transgenes, in four different transgenes, or preferably in one single transgene. In specific embodiments, they are present in one single transgene (i.e., the immunostimulatory cell comprises a transgene comprising: the nucleotide sequence encoding the dominant negative form of an inhibitor of a cell-mediated immune response of the immunostimulatory cell, the nucleotide sequence encoding an immunomodulatory agent, the nucleotide sequence encoding a chimeric antigen receptor (CAR), and the nucleotide sequence encoding a reporter, wherein the transgene is expressible in the immunostimulatory cell to produce the dominant negative form, the immunomodulatory agent, the CAR and the reporter).

In preferred embodiments of the aspect, adjacent occurrences of nucleotide sequences encoding different proteins that are present in the same transgene are separated from each other by a nucleotide sequence encoding a cleavable linker. Nucleotide sequences encoding different cleavable linkers may be used to separate different pairs of adjacent occurrences of nucleotide sequences encoding different proteins that are present in the same transgene. In a specific embodiment, adjacent occurrences of nucleotide sequences encoding different proteins that are present in the same transgene are separated from each other by an internal ribosomal entry site (IRES). In another specific embodiment, adjacent occurrences of nucleotide sequences encoding different proteins that are present in the same transgene are separated from each other by a nucleotide sequence encoding a 2A peptide.

When the nucleotide sequences encoding the different proteins are present in different transgenes, the different transgenes can be present on different vectors or the same vector.

In another aspect, the invention provides immunostimulatory cells comprising a transgene, which transgene comprises a nucleotide sequence encoding a dominant negative form of an inhibitor of a cell-mediated immune response of the immunostimulatory cell, wherein expression of the transgene is under control of an inducible promoter, which inducible promoter is induced upon activation of the immunostimulatory cell (thus, allowing expression of the inhibitor of a cell-mediated immune response only in an activated immunostimulatory cell). In specific embodiments, the inducible promoter is induced by nuclear factor of activated T cells (NFAT) binding. In a specific embodiment, the immunostimulatory cell is a T cell and the promoter is induced by nuclear factor of activated T cells (NFAT) binding.

In various embodiments of the aspect, the immunostimulatory cell further comprises a nucleotide sequence encoding a reporter. The nucleotide sequence encoding a reporter and the nucleotide sequence encoding a dominant negative form can be present in two different transgenes, or preferably in one single transgene. In specific embodiments, they are present in one single transgene (i.e., the immunostimulatory cell comprises a transgene comprising: the nucleotide sequence encoding a dominant negative form of an inhibitor of a cell-mediated immune response of the immunostimulatory cell, and the nucleotide sequence encoding a reporter, wherein the transgene is expressible in the immunostimulatory cell to produce the dominant negative form and the reporter).

In various embodiments of the aspect, the immunostimulatory cell further comprises a nucleotide sequence encoding a chimeric antigen receptor (CAR), wherein the CAR binds to a target antigen associated with a mammalian disease or disorder (i.e., a cancer or an infection with a pathogen). In some embodiments, the immunostimulatory cell further comprises a nucleotide sequence encoding a chimeric antigen receptor (CAR), wherein the CAR binds to a target antigen that is a cancer antigen. In other embodiments, the immunostimulatory cell further comprises a nucleotide sequence encoding a chimeric antigen receptor (CAR), wherein the CAR binds to a target antigen of a pathogen. The nucleotide sequence encoding a CAR and the nucleotide sequence encoding a dominant negative form can be present in two different transgenes, or preferably in one single transgene. In specific embodiments, they are present in one single transgene (i.e., the immunostimulatory cell comprises a transgene comprising: the nucleotide sequence encoding a dominant negative form of an inhibitor of a cell-mediated immune response of the immunostimulatory cell and the nucleotide sequence encoding a chimeric antigen receptor (CAR), wherein the transgene is expressible in the immunostimulatory cell to produce the dominant negative form and the CAR). In specific embodiments, the immunostimulatory cell further comprises a nucleotide sequence encoding a reporter. The nucleotide sequence encoding a reporter, the nucleotide sequence encoding a CAR, and the nucleotide sequence encoding a dominant negative form can be present in two different transgenes, in three different transgenes, or preferably in one single transgene. In specific embodiments, they are present in one single transgene (i.e., the immunostimulatory cell comprises a transgene comprising: the nucleotide sequence encoding a dominant negative form of an inhibitor of a cell-mediated immune response of the immunostimulatory cell, the nucleotide sequence encoding a chimeric antigen receptor (CAR), and the nucleotide sequence encoding a reporter, wherein the transgene is expressible in the immunostimulatory cell to produce the dominant negative form, the CAR and the reporter).

In preferred embodiments of the aspect, adjacent occurrences of nucleotide sequences encoding different proteins that are present in the same transgene are separated from each other by a nucleotide sequence encoding a cleavable linker. Nucleotide sequences encoding different cleavable linkers may be used to separate different pairs of adjacent occurrences of nucleotide sequences encoding different proteins that are present in the same transgene. In a specific embodiment, adjacent occurrences of nucleotide sequences encoding different proteins that are present in the same transgene are separated from each other by an internal ribosomal entry site (IRES). In another specific embodiment, adjacent occurrences of nucleotide sequences encoding different proteins that are present in the same transgene are separated from each other by a nucleotide sequence encoding a 2A peptide.

When the nucleotide sequences encoding the different proteins are present in different transgenes, the different transgenes can be present on different vectors or the same vector.

In another aspect, the invention provides immunostimulatory cells comprising a transgene, which transgene comprises a nucleotide sequence encoding an immunomodulatory agent, wherein expression of the transgene is under control of an inducible promoter, which inducible promoter is induced upon activation of the immunostimulatory cell (thus, allowing expression of the immunomodulatory agent only in an activated immunostimulatory cell), wherein the immunomodulatory agent is a single chain variable fragment (scFv) or peptide antibody, which immunomodulatory agent binds to and inhibits an immune checkpoint inhibitor. In specific embodiments, the inducible promoter is induced by nuclear factor of activated T cells (NFAT) binding. In a specific embodiment, the immunostimulatory cell is a T cell and the inducible promoter is induced by nuclear factor of activated T cells (NFAT) binding.

In various embodiments of the aspect, the immunostimulatory cell further comprises a nucleotide sequence encoding a reporter. The nucleotide sequence encoding a reporter and the nucleotide sequence encoding an immunomodulatory agent can be present in two different transgenes, or preferably in one single transgene. In specific embodiments, they are present in one single transgene (i.e., the immunostimulatory cell comprises a transgene comprising: the nucleotide sequence encoding an immunomodulatory agent and the nucleotide sequence encoding a reporter, wherein the transgene is expressible in the immunostimulatory cell to produce the immunomodulatory agent and the reporter).

In various embodiments of the aspect, the immunostimulatory cell further comprises a nucleotide sequence encoding a chimeric antigen receptor (CAR), wherein the CAR binds to a target antigen associated with a mammalian disease or disorder (i.e., a cancer or an infection with a pathogen). In some embodiments, the immunostimulatory cell further comprises a nucleotide sequence encoding a chimeric antigen receptor (CAR), wherein the CAR binds to a target antigen that is a cancer antigen. In other embodiments, the immunostimulatory cell further comprises a nucleotide sequence encoding a chimeric antigen receptor (CAR), wherein the CAR binds to a target antigen of a pathogen. The nucleotide sequence encoding a CAR and the nucleotide sequence encoding an immunomodulatory agent can be present in two different transgenes, or preferably in one single transgene. In specific embodiments, they are present in one single transgene (i.e., the immunostimulatory cell comprises a transgene comprising: the nucleotide sequence encoding an immunomodulatory agent and the nucleotide sequence encoding a chimeric antigen receptor (CAR), wherein the transgene is expressible in the immunostimulatory cell to produce the immunomodulatory agent and the CAR). In specific embodiments, the immunostimulatory cell further comprises a nucleotide sequence encoding a reporter. The nucleotide sequence encoding a reporter, the nucleotide sequence encoding a CAR, and the nucleotide sequence encoding an immunomodulatory agent can be present in two different transgenes, in three different transgenes, or preferably in one single transgene. In specific embodiments, they are present in one single transgene (i.e., the immunostimulatory cell comprises a transgene comprising: the nucleotide sequence encoding an immunomodulatory agent, the nucleotide sequence encoding a chimeric antigen receptor (CAR), and the nucleotide sequence encoding a reporter, wherein the transgene is expressible in the immunostimulatory cell to produce the immunomodulatory agent, the CAR and the reporter).

In preferred embodiments of the aspect, adjacent occurrences of nucleotide sequences encoding different proteins that are present in the same transgene are separated from each other by a nucleotide sequence encoding a cleavable linker. Nucleotide sequences encoding different cleavable linkers may be used to separate different pairs of adjacent occurrences of nucleotide sequences encoding different proteins that are in the same transgene. In a specific embodiment, adjacent occurrences of nucleotide sequences encoding different proteins that are present in the same transgene are separated from each other by an internal ribosomal entry site (IRES). In another specific embodiment, adjacent occurrences of nucleotide sequences encoding different proteins that are present in the same transgene are separated from each other by a nucleotide sequence encoding a 2A peptide.

When the nucleotide sequences encoding the different proteins are present in different transgenes, the different transgenes can be present on different vectors or the same vector.

In another aspect, the invention provides immunostimulatory cells comprising: (a) a nucleotide sequence encoding a dominant negative form of an inhibitor of a cell-mediated immune response of the immunostimulatory cell, and (b) a nucleotide sequence encoding an immunomodulatory agent, wherein the immunomodulatory agent is a single chain variable fragment (scFv) or peptide antibody, which immunomodulatory agent binds to and inhibits an immune checkpoint inhibitor, wherein the immune checkpoint inhibitor is different from the inhibitor of a cell-mediated immune response, and wherein expression of the dominant negative form and/or the immunomodulatory agent is under control of an inducible promoter, which inducible promoter is induced upon activation of the immunostimulatory cell (thus, allowing expression of the dominant negative form and/or the immunomodulatory agent only in an activated immunostimulatory cell). In specific embodiments, the inducible promoter is induced by nuclear factor of activated T cells (NFAT) binding. In a specific embodiment, the immunostimulatory cell is a T cell and the inducible promoter is induced by nuclear factor of activated T cells (NFAT) binding.

The nucleotide sequence encoding a dominant negative form and the nucleotide sequence encoding an immunomodulatory agent can be present in two different transgenes or preferably in one single transgene. In specific embodiments, they are present in one single transgene (i.e., the immunostimulatory cells comprise a transgene comprising: the nucleotide sequence encoding a dominant negative form of an inhibitor of a cell-mediated immune response of the immunostimulatory cell, and the nucleotide sequence encoding an immunomodulatory agent).

In various embodiments of the aspect, the immunostimulatory cell further comprises a nucleotide sequence encoding a reporter. The nucleotide sequence encoding a reporter, the nucleotide sequence encoding a dominant negative form, and the nucleotide sequence encoding an immunomodulatory agent can be present in two different transgenes, in three different transgenes, or preferably in one single transgene. In specific embodiments, they are present in one single transgene (i.e., the immunostimulatory cell comprises a transgene comprising: the nucleotide sequence encoding a dominant negative form of an inhibitor of a cell-mediated immune response of the immunostimulatory cell, the nucleotide sequence encoding an immunomodulatory agent, and the nucleotide sequence encoding a reporter, wherein the transgene is expressible in the immunostimulatory cell to produce the dominant negative form, the immunomodulatory agent, and the reporter).

In various embodiments of the aspect, the immunostimulatory cell further comprises a nucleotide sequence encoding a chimeric antigen receptor (CAR), wherein the CAR binds to a target antigen associated with a mammalian disease or disorder (i.e., a cancer or an infection with a pathogen). In some embodiments, the immunostimulatory cell further comprises a nucleotide sequence encoding a chimeric antigen receptor (CAR), wherein the CAR binds to a target antigen that is a cancer antigen. In other embodiments, the immunostimulatory cell further comprises a nucleotide sequence encoding a chimeric antigen receptor (CAR), wherein the CAR binds to a target antigen of a pathogen. The nucleotide sequence encoding a CAR, the nucleotide sequence encoding a dominant negative form, and the nucleotide sequence encoding an immunomodulatory agent can be present in two different transgenes, in three different transgenes, or preferably in one single transgene. In specific embodiments, they are present in one single transgene (i.e., the immunostimulatory cell comprises a transgene comprising: the nucleotide sequence encoding the nucleotide sequence encoding a dominant negative form of an inhibitor of a cell-mediated immune response of the immunostimulatory cell, the nucleotide sequence encoding an immunomodulatory agent, and the nucleotide sequence encoding a chimeric antigen receptor (CAR), wherein the transgene is expressible in the immunostimulatory cell to produce the dominant negative form, the immunomodulatory agent, and the CAR). In specific embodiments, the immunostimulatory cell further comprises a nucleotide sequence encoding a reporter. The nucleotide sequence encoding a reporter, the nucleotide sequence encoding a CAR, the nucleotide sequence encoding a dominant negative form, and the nucleotide sequence encoding an immunomodulatory agent can be present in two different transgenes, in three different transgenes, in four different transgenes, or preferably in one single transgene. In specific embodiments, they are present in one single transgene (i.e., the immunostimulatory cell comprises a transgene comprising: the nucleotide sequence encoding a dominant negative form of an inhibitor of a cell-mediated immune response of the immunostimulatory cell, the nucleotide sequence encoding an immunomodulatory agent, the nucleotide sequence encoding a chimeric antigen receptor (CAR), and the nucleotide sequence encoding a reporter, wherein the transgene is expressible in the immunostimulatory cell to produce the dominant negative form, the immunomodulatory agent, the CAR and the reporter).

In preferred embodiments of the aspect, adjacent occurrences of nucleotide sequences encoding different proteins that are present in the same transgene are separated from each other by a nucleotide sequence encoding a cleavable linker. Nucleotide sequences encoding different cleavable linkers may be used to separate different pairs of adjacent occurrences of nucleotide sequences encoding different proteins that are present in the same transgene. In a specific embodiment, adjacent occurrences of nucleotide sequences encoding different proteins that are present in the same transgene are separated from each other by an internal ribosomal entry site (IRES). In another specific embodiment, adjacent occurrences of nucleotide sequences encoding different proteins that are present in the same transgene are separated from each other by a nucleotide sequence encoding a 2A peptide.

When the nucleotide sequences encoding the different proteins are present in different transgenes, the different transgenes can be present on different vectors or the same vector.

In another aspect, the invention provides immunostimulatory cells in one or more transgenes comprising: (a) a nucleotide sequence encoding a chimeric antigen receptor (CAR), wherein the CAR binds to a target antigen associated with a mammalian disease or disorder (i.e., a cancer or an infection with a pathogen), (b) a nucleotide sequence encoding a dominant negative form of an inhibitor of a cell-mediated immune response of the immunostimulatory cell, and (c) a nucleotide sequence encoding a membrane bound form of interleukin 12 (membrane IL-12). Membrane IL-12 has reduced side effects as compared with secreted IL-12. In a specific embodiment, the membrane IL-12 is as described in Pan et al., Molecular Therapy 20(5): 927-937 (2012). In certain embodiments, the membrane IL-12 comprises a p40 subunit and a p35 subunit separated by a linker, wherein the p35 subunit is fused to a transmembrane domain (for example, a transmembrane domain of CD8). The linker can by any peptide sequence known in the art to be used to link a heavy chain variable region and a light chain variable region in a scFv. In specific embodiments, the immunostimulatory cell is a T cell, a macrophage, a dendritic cell, or a Natural Killer (NK) cell.

The nucleotide sequence encoding a CAR, the nucleotide sequence encoding a dominant negative form, and the nucleotide sequence encoding a membrane IL-12 can be present in two different transgenes, in three different transgenes, or in one single transgene.

In specific embodiments of the aspect, the immunostimulatory cells comprise a transgene comprising: (a) the nucleotide sequence encoding a chimeric antigen receptor (CAR), (b) the nucleotide sequence encoding a dominant negative form of an inhibitor of a cell-mediated immune response of the immunostimulatory cell, and (c) the nucleotide sequence encoding a membrane bound form of interleukin 12 (membrane IL-12), wherein expression of the transgene is under control of a promoter such that the transgene is expressible in the immunostimulatory cell to produce the CAR, the dominant negative form and the membrane IL-12.

In specific embodiments of the aspect, the immunostimulatory cells comprise: (1) a first transgene, which first transgene comprises: (a) the nucleotide sequence encoding a chimeric antigen receptor (CAR), and (b) the nucleotide sequence encoding a dominant negative form of an inhibitor of a cell-mediated immune response of the immunostimulatory cell, and (2) a second transgene, which second transgene comprises (c) the nucleotide sequence encoding a membrane bound form of interleukin 12 (membrane IL-12), wherein expression of the first transgene is under control of a promoter (for example, a constitutive promoter) such that the first transgene is expressible in the immunostimulatory cell to produce the CAR and the dominant negative form, and wherein expression of the second transgene is under control of an inducible promoter, which inducible promoter is induced upon activation of the immunostimulatory cell. In specific embodiments, the inducible promoter is induced by nuclear factor of activated T cells (NFAT) binding. In a specific embodiment, the immunostimulatory cell is a T cell and the inducible promoter is induced by nuclear factor of activated T cells (NFAT) binding.

In specific embodiments of the aspect, the immunostimulatory cells comprise: (1) a first transgene, which first transgene comprises (a) the nucleotide sequence encoding a chimeric antigen receptor (CAR), (2) a second transgene, which second transgene comprises (b) the nucleotide sequence encoding a dominant negative form of an inhibitor of a cell-mediated immune response of the immunostimulatory cell, and (3) a third transgene, which third transgene comprises (c) the nucleotide sequence encoding a membrane bound form of interleukin 12 (membrane IL-12), wherein expression of the third transgene is under control of an inducible promoter, which inducible promoter is induced upon activation of the immunostimulatory cell. In specific embodiments, the inducible promoter is induced by nuclear factor of activated T cells (NFAT) binding. In a specific embodiment, the immunostimulatory cell is a T cell and the inducible promoter is induced by nuclear factor of activated T cells (NFAT) binding. In a specific embodiment, the first and the second transgenes are under control of constitutive promoters.

In various embodiments of the aspect, the immunostimulatory cell further comprises a nucleotide sequence encoding a reporter. The nucleotide sequence encoding a reporter can be present in the same transgene as or on a different transgene from the transgene(s) comprising the nucleotide sequence encoding a CAR, the nucleotide sequence encoding a dominant negative form, and/or the nucleotide sequence encoding a membrane IL-12. If the nucleotide sequence encoding a CAR, the nucleotide sequence encoding a dominant negative form, and the nucleotide sequence encoding a membrane IL-12 are present in one single transgene, preferably the nucleotide sequence encoding a reporter is also present in the same transgene. If the nucleotide sequence encoding a CAR, the nucleotide sequence encoding a dominant negative form, and the nucleotide sequence encoding a membrane IL-12 are present in two or three transgenes (in particular if they are each present on a different vector), preferably each of the two or three transgenes comprises a nucleotide sequence encoding a different reporter (for example, a red fluorescence reporter on one transgene and a green fluorescence reporter on a different transgene).

In preferred embodiments of the aspect, adjacent occurrences of nucleotide sequences encoding different proteins that are present in the same transgene are separated from each other by a nucleotide sequence encoding a cleavable linker. Nucleotide sequences encoding different cleavable linkers may be used to separate different pairs of adjacent occurrences of nucleotide sequences encoding different proteins that are present in the same transgene. In a specific embodiment, adjacent occurrences of nucleotide sequences encoding different proteins that are present in the same transgene are separated from each other by an internal ribosomal entry site (IRES). In another specific embodiment, adjacent occurrences of nucleotide sequences encoding different proteins that are present in the same transgene are separated from each other by a nucleotide sequence encoding a 2A peptide.

When the nucleotide sequences encoding the different proteins are present in different transgenes, the different transgenes can be present on different vectors or the same vector.

In another aspect, the invention provides immunostimulatory cells comprising: (1) in one or more transgenes: (a) a nucleotide sequence encoding a chimeric antigen receptor (CAR), wherein the CAR binds to a target antigen associated with a mammalian disease or disorder, (b) a nucleotide sequence encoding a dominant negative form of an inhibitor of a cell-mediated immune response of the immunostimulatory cell, that is a receptor-synthetic Notch fusion protein comprising (i) an extracellular domain of the inhibitor of a cell-mediated immune response of the immunostimulatory cell, (ii) the transmembrane core domain of Notch (which mediates control of proteolysis) C-terminal to the extracellular domain, and (iii) a transcription factor C-terminal to the transmembrane core domain of Notch, and (2) in a different transgene (c) a nucleotide sequence encoding a membrane bound form of interleukin 12 (membrane IL-12), wherein expression of the membrane IL-12 is under control of an inducible promoter, which inducible promoter is induced upon binding of the transcription factor, and wherein the transcription factor is cleaved from the receptor-synthetic Notch fusion protein intracellularly upon binding of the extracellular domain to its ligand. The receptor-synthetic Notch fusion protein can be generated as described in, for example, Example 1, Morsut et al., Cell, 164(4): 780-791 (2016), or Roybal et al., Cell, 167: 419-432 (2016). Membrane IL-12 has reduced side effects as compared with secreted IL-12. In a specific embodiment, the membrane IL-12 is as described in Pan et al., Molecular Therapy 20(5): 927-937 (2012). In certain embodiments, the membrane IL-12 comprises a p40 subunit and a p35 subunit separated by a linker, wherein the p35 subunit is fused to a transmembrane domain (for example, the transmembrane domain of CD8). The linker can by any peptide sequence known in the art to be used to link a heavy chain variable region and a light chain variable region in a scFv. In specific embodiments, the immunostimulatory cell is a T cell, a macrophage, a dendritic cell, or a Natural Killer (NK) cell.

The nucleotide sequence encoding a CAR and the nucleotide sequence encoding a receptor-synthetic Notch fusion protein can be present in two different transgenes, or preferably in one single transgene.

In specific embodiments of the aspect, the immunostimulatory cells comprise: (1) a first transgene, which first transgene comprises: (a) the nucleotide sequence encoding a chimeric antigen receptor (CAR), and (b) the nucleotide sequence encoding a receptor-synthetic Notch fusion protein, and (2) a second transgene, which second transgene comprises (c) the nucleotide sequence encoding a membrane bound form of interleukin 12 (membrane IL-12), wherein expression of the first transgene is under control of a promoter (for example, a constitutive promoter) such that the first transgene is expressible in the immunostimulatory cell to produce the CAR and the receptor-synthetic Notch fusion protein, and wherein expression of the membrane IL-12 is under control of an inducible promoter, which inducible promoter is induced upon binding of the transcription factor, and wherein the transcription factor is cleaved from the receptor-synthetic Notch fusion protein intracellularly upon binding of the extracellular domain to its ligand.

In specific embodiments of the aspect, the immunostimulatory cells comprise: (1) a first transgene, which first transgene comprises (a) the nucleotide sequence encoding a chimeric antigen receptor (CAR), (2) a second transgene, which second transgene comprises (b) the nucleotide sequence encoding a receptor-synthetic Notch fusion protein, and (3) a third transgene, which third transgene comprises (c) the nucleotide sequence encoding a membrane bound form of interleukin 12 (membrane IL-12), wherein expression of the membrane IL-12 is under control of an inducible promoter, which inducible promoter is induced upon binding of the transcription factor, and wherein the transcription factor is cleaved from the receptor-synthetic Notch fusion protein intracellularly upon binding of the extracellular domain to its ligand.

In various embodiments of the aspect, the immunostimulatory cell further comprises a nucleotide sequence encoding a reporter. The nucleotide sequence encoding a reporter can be present in the same transgene as or on a different transgene from the transgene(s) comprising the nucleotide sequence encoding a CAR, the nucleotide sequence encoding a receptor-synthetic Notch fusion protein, and/or the nucleotide sequence encoding a membrane IL-12. If the nucleotide sequence encoding a CAR and the nucleotide sequence encoding a receptor-synthetic Notch fusion protein are present in one single transgene, preferably the nucleotide sequence encoding a reporter is also present in the same transgene. If the nucleotide sequence encoding a CAR, the nucleotide sequence encoding a receptor-synthetic Notch fusion protein, and the nucleotide sequence encoding a membrane IL-12 are present in three transgenes (in particular if they are each present on a different vector), preferably each of the three transgenes comprises a nucleotide sequence encoding a different reporter (for example, a red fluorescence reporter on the first transgene, a green fluorescence reporter on the second transgene, and a blue fluorescence reporter on the third transgene).

In preferred embodiments of the aspect, adjacent occurrences of nucleotide sequences encoding different proteins that are present in the same transgene are separated from each other by a nucleotide sequence encoding a cleavable linker. Nucleotide sequences encoding different cleavable linkers may be used to separate different pairs of adjacent occurrences of nucleotide sequences encoding different proteins that are present in the same transgene. In a specific embodiment, adjacent occurrences of nucleotide sequences present in the same transgene are separated from each other by an internal ribosomal entry site (IRES). In another specific embodiment, adjacent occurrences of nucleotide sequences present in the same transgene are separated from each other by a nucleotide sequence encoding a 2A peptide.

When the nucleotide sequences encoding the different proteins are present in different transgenes, the different transgenes can be present on different vectors or the same vector.

In another aspect, the invention provides immunostimulatory cells comprising in one or more transgenes: (a) a nucleotide sequence encoding a dominant negative form of an inhibitor of a cell-mediated immune response of the immunostimulatory cell, and (b) a nucleotide sequence encoding interleukin 12 (IL-12), wherein IL-12 when expressed by the immunostimulatory is secreted from the immunostimulatory cell. In specific embodiments, the immunostimulatory cell is a T cell, a macrophage, a dendritic cell, or a Natural Killer (NK) cell.

The nucleotide sequence encoding a dominant negative form and the nucleotide sequence encoding IL-12 can be present in two different transgenes or in one single transgene.

In specific embodiments of the aspect, the immunostimulatory cell comprises a transgene comprising: (a) the nucleotide sequence encoding a dominant negative form of an inhibitor of a cell-mediated immune response of the immunostimulatory cell, and (b) the nucleotide sequence encoding interleukin 12 (IL-12), wherein the nucleotide sequence encoding the dominant negative form and the nucleotide sequence encoding the IL-12 are separated by an internal ribosome entry site (IRES), wherein expression of the transgene is under control of a promoter (for example, a constitutive promoter) such that the transgene is expressible in the immunostimulatory cell to produce the dominant negative form and the IL-12.

In specific embodiments of the aspect, the immunostimulatory cell comprises: (1) a first transgene comprising (a) the nucleotide sequence encoding a dominant negative form of an inhibitor of a cell-mediated immune response of the immunostimulatory cell, and (2) a second transgene comprising (b) the nucleotide sequence encoding interleukin 12 (IL-12), wherein expression of the dominant negative form is under control of a promoter (for example, a constitutive promoter) such that the first transgene is expressible in the immunostimulatory cell to produce the dominant negative form, wherein expression of the IL-12 is under control of an inducible promoter, which inducible promoter is induced upon activation of the immunostimulatory cell, and wherein the IL-12 when expressed by the immunostimulatory is secreted from the immunostimulatory cell. In specific embodiments, the inducible promoter is induced by nuclear factor of activated T cells (NFAT) binding. In a specific embodiment, the immunostimulatory cell is a T cell and the inducible promoter is induced by nuclear factor of activated T cells (NFAT) binding.

In various embodiments of the aspect, the immunostimulatory cell further comprises a nucleotide sequence encoding a reporter. The nucleotide sequence encoding a reporter can be present in the same transgene as or on a different transgene from the transgene(s) comprising the nucleotide sequence encoding a dominant negative form and/or the nucleotide sequence encoding a secreted IL-12. If the nucleotide sequence encoding a dominant negative form and the nucleotide sequence encoding a secreted IL-12 are present in one single transgene, preferably the nucleotide sequence encoding a reporter is also present in the same transgene. If the nucleotide sequence encoding a dominant negative form and the nucleotide sequence encoding a secreted IL-12 are present in two transgenes (and in particular if the two transgenes are present on two vectors), preferably each of the two transgenes comprises a nucleotide sequence encoding a different reporter (for example, a red fluorescence reporter on one transgene and a green fluorescence reporter on the other transgene).

In preferred embodiments of the aspect, adjacent occurrences of nucleotide sequences encoding different proteins that are present in the same transgene are separated from each other by a nucleotide sequence encoding a cleavable linker. Nucleotide sequences encoding different cleavable linkers may be used to separate different pairs of adjacent occurrences of nucleotide sequences encoding different proteins that are present in the same transgene. In a specific embodiment, adjacent occurrences of nucleotide sequences present in the same transgene are separated from each other by an internal ribosomal entry site (IRES). In another specific embodiment, adjacent occurrences of nucleotide sequences present in the same transgene are separated from each other by a nucleotide sequence encoding a 2A peptide.

When the nucleotide sequences encoding the different proteins are present in different transgenes, the different transgenes can be present on different vectors or the same vector.

In a preferred embodiment of the invention, the immunomodulatory agent is a scFv that binds to an immune checkpoint inhibitor. Preferably, the scFv binds to and inhibits (e.g., by blocking ligand binding or inhibiting signaling of) the immune checkpoint inhibitor.

In a specific embodiment of the invention, the immunomodulatory agent is a peptide antibody that binds to an immune checkpoint inhibitor. Preferably, the peptide antibody binds to and inhibits (e.g., by blocking ligand binding or inhibiting signaling of) the immune checkpoint inhibitor. A peptide antibody that binds to an immune checkpoint inhibitor can be an antigen-binding fragment (for example, a variable region or part of a variable region) of an antibody that binds to and inhibits the immune checkpoint inhibitor, or a ligand that binds to and inhibits the immune checkpoint inhibitor (when the immune checkpoint inhibitor is a receptor).

In specific embodiments of the invention, the immunomodulatory agent is secreted from the immunostimulatory cell.

In specific embodiments of the invention, the immune checkpoint inhibitor to which the immunomodulatory agent binds is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a specific embodiment, the immune checkpoint inhibitor to which the immunomodulatory agent binds is TIM-3. In another specific embodiment, the immune checkpoint inhibitor to which the immunomodulatory agent binds is LAG-3.

In some embodiments of the invention, the inhibitor of a cell-mediated immune response is an immune checkpoint inhibitor. In specific embodiments, the inhibitor of a cell-mediated immune response is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160. In a preferred embodiment, the inhibitor of a cell-mediated immune response is PD-1.

In other embodiments of the invention, the inhibitor of a cell-mediated immune response is transforming growth factor β (TGF- (3) receptor.

In specific embodiments of the invention, the dominant negative form of the inhibitor of a cell-mediate immune response is expressed as a membrane protein on the surface of the immunostimulatory cell.

The reporter as described herein can be any protein that can be used as an indication of gene expression in the immunostimulatory cell, and makes the cells expressing it easily identified, measured, and/or selected. Preferably, the reporter is non-toxic to the immunostimulatory cell, and its expression can be detected in live cells. In specific embodiments, the reporter is a fluorescent protein, such as red fluorescent protein (for example, mCherry, RFP, tdTomato, or DsRed), green fluorescent protein (for example, GFP or EGFR), green fluorescent protein (for example, EBFP, EBFP2, Azurite, or mKalamal), cyan fluorescent protein (for example, ECFP, Cerulean, or CyPet), or yellow fluorescent protein (for example, YFP, Citrine, Venus, or YPet). In a specific embodiment, the reporter is mCherry. In a specific embodiment, the reporter is low-affinity nerve growth factor receptor (LNGFR). In a specific embodiment, the reporter is a truncated mutant of LNGFR (for example, a mutant LNGFR lacking the cytoplasmic domain, such as one described in Lauer et al., Cancer Gene Ther. 7:430-437 (2000)).

The “cleavable linker” as used herein includes any linker sequence that allows for bicistronic or multicistronic expression from the same promoter, such as a 2A peptide, by providing for separation of the multiple proteins encoded by the RNA molecule transcribed under control of the promoter. Such separation can occur regardless of mechanism, e.g., by cleavage, ribosome skipping, separate ribosome entry site, etc. Non-limiting exemplary 2A peptides that can be used in the invention include P2A peptides, T2A peptides, E2A peptides, and F2A peptides (see, for example, Szymczak et al., Expert Opin. Biol. Therapy 5(5):627-638 (2005), Ibrahimi et al., Hum Gene Ther. 20(8):845-860 (2009), Kim et al., PLoS One 6(4):e18556 (2011)). Alternatively, an internal ribosomal entry site (IRES) can be used as the nucleotide sequence encoding the cleavable linker to allow for bicistronic or multicistronic expression from the same promoter. As will be clear, an IRES as used herein in the context of a transgene or any DNA nucleic acid sequence is the DNA sequence corresponding to an RNA IRES element.

The immunostimulatory cells can be used to enhance or provide an immune response against a target antigen such as a cancer antigen or an antigen of a pathogen. Preferably, the immunostimulatory cells are derived from a human (are of human origin prior to being made recombinant) (and human-derived immunostimulatory cells are particularly preferred for administration to a human in the methods of treatment of the invention).

The immunostimulatory cells of the invention are immune cells that stimulate or promote immune response or their precursor cells, such as cells of the lymphoid lineage. Non-limiting examples of cells of the lymphoid lineage that can be used as immunostimulatory cells include T cells and Natural Killer (NK) cells. In a preferred embodiment, the immunostimulatory cells are T cells. T cells express the T cell receptor (TCR), with most cells expressing α and β chains and a smaller population expressing γ and δ chains. T cells useful as immunostimulatory cells of the invention can be CD4⁺ or CD8⁺ and can include, but are not limited to, T helper cells (CD4⁺), cytotoxic T cells (also referred to as cytotoxic T lymphocytes, CTL; CD8⁺ T cells), and memory T cells, including central memory T cells, stem-cell-like memory T cells (or stem-like memory T cells), and effector memory T cells, for example, TEM cells and T_{EMRA} (CD45RA⁺) cells, natural killer T cells, mucosal associated invariant T cells (MAIT), and γδ T cells. In a specific embodiment, the T cell is CD4⁺. In another specific embodiment, the T cell is CD8⁺. In another specific embodiment, the T cell is a CTL. In a specific embodiment, the immunostimulatory cell is a NK cell. Other exemplary immunostimulatory cells include, but are not limited to, macrophages, antigen presenting cells (APCs) such as dendritic cells, or any immune cell that expresses an inhibitor of a cell-mediated immune response, for example, an immune checkpoint inhibitor pathway receptor, e.g., PD-1 (in such instance expression of the dominant negative form in the cell inhibits the inhibitor of the cell-mediated immune response to promote sustained activation of the cell). Precursor cells of immune cells that can be used according to the invention are, by way of example, hematopoietic stem and/or progenitor cells. Hematopoietic stem and/or progenitor cells can be derived from bone marrow, umbilical cord blood, adult peripheral blood after cytokine mobilization, and the like, by methods known in the art. Particularly useful precursor cells are those that can differentiate into the lymphoid lineage, for example, hematopoietic stem cells or progenitor cells of the lymphoid lineage.

Immune cells and precursor cells thereof can be isolated by methods well known in the art, including commercially available isolation methods (see, for example, Rowland-Jones et al., Lymphocytes: A Practical Approach, Oxford University Press, New York (1999)). Sources for the immune cells or precursor cells thereof include, but are not limited to, peripheral blood, umbilical cord blood, bone marrow, or other sources of hematopoietic cells. Various techniques can be employed to separate the cells to isolate or enrich for desired immune cells. For instance, negative selection methods can be used to remove cells that are not the desired immune cells. Additionally, positive selection methods can be used to isolate or enrich for desired immune cells or precursor cells thereof, or a combination of positive and negative selection methods can be employed. Monoclonal antibodies (MAbs) are particularly useful for identifying markers associated with particular cell lineages and/or stages of differentiation for both positive and negative selections. If a particular type of cell is to be isolated, for example, a particular type of T cell, various cell surface markers or combinations of markers, including but not limited to, CD3, CD4, CD8, CD34 (for hematopoietic stem and progenitor cells) and the like, can be used to separate the cells, as is well known in the art (see Kearse, T Cell Protocols: Development and Activation, Humana Press, Totowa N.J. (2000); De Libero, T Cell Protocols, Vol. 514 of Methods in Molecular Biology, Humana Press, Totowa N.J. (2009)).

Procedures for separation of cells include, but are not limited to, density gradient centrifugation, coupling to particles that modify cell density, magnetic separation with antibody-coated magnetic beads, affinity chromatography; cytotoxic agents joined to or used in conjunction with a monoclonal antibody (mAb), including, but not limited to, complement and cytotoxins, and panning with an antibody attached to a solid matrix, for example, a plate or chip, elutriation, flow cytometry, or any other convenient technique (see, for example, Recktenwald et al., Cell Separation Methods and Applications, Marcel Dekker, Inc., New York (1998)).

The immune cells or precursor cells thereof can be autologous or non-autologous to the subject to which they are administered in the methods of treatment of the invention. Autologous cells are isolated from the subject to which the immunostimulatory cells are to be administered. Optionally, the cells can be obtained by leukapheresis, where leukocytes are selectively removed from withdrawn blood, made recombinant, and then retransfused into the donor. Alternatively, allogeneic cells from a non-autologous donor that is not the subject can be used. In the case of a non-autologous donor, the cells are typed and matched for human leukocyte antigen (HLA) to determine an appropriate level of compatibility, as is well known in the art. For both autologous and non-autologous cells, the cells can optionally be cryopreserved until ready to be used for genetic manipulation and/or administration to a subject using methods well known in the art.

Various methods for isolating immune cells that can be used for recombinant engineering have been described previously, and can be used, including but not limited to, using peripheral donor lymphocytes (Sadelain et al., Nat. Rev. Cancer 3:35-45 (2003); Morgan et al., Science 314:126-129 (2006), using lymphocyte cultures derived from tumor infiltrating lymphocytes (TILs) in tumor biopsies (Panelli et al., J Immunol. 164:495-504 (2000); Panelli et al., J. Immunol. 164:4382-4392 (2000)), and using selectively in vitro-expanded antigen-specific peripheral blood leukocytes employing artificial antigen-presenting cells (AAPCs) or dendritic cells (Dupont et al., Cancer Res. 65:5417-5427 (2005); Papanicolaou et al., Blood 102:2498-2505 (2003)). In the case of using stem cells, the cells can be isolated by methods well known in the art (see, for example, Klug et al., Hematopoietic Stem Cell Protocols, Humana Press, N.J. (2002); Freshney et al., Culture of Human Stem Cells, John Wiley & Sons (2007)).

The immunostimulatory cells can be genetically engineered for recombinant expression to introduce the one or more nucleotide sequences described above by methods well known in the art.

In certain embodiments of the invention, the immunostimulatory cells according to the invention (for example, T cells) recognize and are sensitized to a target antigen associated with a mammalian disease or disorder (i.e., cancer or infection with a pathogen). In some embodiments, the immunostimulatory cells according to the invention (for example, T cells) recognize and are sensitized to a target antigen that is a cancer antigen. In other embodiments, the immunostimulatory cells according to the invention (for example, T cells) recognize and are sensitized to a target antigen of a pathogen. Such immunostimulatory cells (for example, T cells) can but need not express a CAR that binds to a target antigen, since the cells already are target antigen-specific so that their immune response (for example, cytotoxicity) is stimulated specifically by such target antigen (generally in the form of a cell expressing the target antigen on its cell surface). Such immunostimulatory cells (for example, T cells) that recognize and are sensitized to a target antigen can be obtained by known methods, by way of example, in vitro sensitization methods using naive T cells (see, for example, Wolfl et al., Nat. Protocols 9:950-966 (2014)) or hematopoietic progenitor cells (see van Lent et al., J. Immunol. 179:4959-4968 (2007)); or obtained from a subject that has been exposed to and is mounting an immune response against the target antigen. Methods for isolating an antigen-specific T cell from a subject are well known in the art. Such methods include, but are not limited to, a cytokine capture system or cytokine secretion assay, which is based on the secretion of cytokines from antigen stimulated T cells that can be used to identify and isolate antigen-specific, and expansion of cells in vitro (see Assenmacher et al., Cytometric Cytokine Secretion Assay, in Analyzing T Cell Responses: How to Analyze Cellular Immune Responses Against Tumor Associated Antigens, Nagorsen et al., eds., Chapter 10, pp. 183-195, Springer, The Netherlands (2005); Haney et al., J. Immunol. Methods 369:33-41 (2011); Bunos et al., Vox Sanguinis DOI: 10.1111/vox.12291 (2015); Montes et al., Clin. Exp. Immunol. 142:292-302 (2005); Adusumilli et al., Sci Transl Med. 6:261ra151 (2014)). Such cytokines include, but are not limited to interferon-γ and tumor necrosis factor-α. The antigen-specific T cells can be isolated using well known techniques as described above for isolating immune cells, which include, but are not limited to, flow cytometry, magnetic beads, panning on a solid phase, and so forth. Antigen-specific T cell isolation techniques are also commercially available, which can be used or adapted for clinical applications (see, for example, Miltenyi Biotec, Cambridge, Mass.; Proimmune, Oxford, UK; and the like).

In an embodiment where target antigen sensitized immunostimulatory cells (for example, T cells) are used, and wherein such cells are obtained by in vitro sensitization, the sensitization can occur before or after the immunostimulatory cells (for example, T cells) are genetically engineered to introduce the one or more nucleotide sequences described above. In an embodiment where the sensitized immunostimulatory cells (for example T cells) are isolated from in vivo sources, it will be self-evident that genetic engineering occurs of the already-sensitized immunostimulatory cells (for example, T cells).

The immune cells or precursor cells thereof can be subjected to conditions that favor maintenance or expansion of the immune cells or precursor cells thereof (see Kearse, T Cell Protocols: Development and Activation, Humana Press, Totowa N.J. (2000); De Libero, T Cell Protocols, Vol. 514 of Methods in Molecular Biology, Humana Press, Totowa N.J. (2009); Parente-Pereira et al., J. Biol. Methods 1(2) e7 (doi 10.14440/jbm.2014.30) (2014); Movassagh et al., Hum. Gene Ther. 11:1189-1200 (2000); Rettig et al., Mol. Ther. 8:29-41 (2003); Agarwal et al., J. Virol. 72:3720-3728 (1998); Pollok et al., Hum. Gene Ther. 10:2221-2236 (1999); Quinn et al., Hum. Gene Ther. 9:1457-1467 (1998); see also commercially available methods such as Dynabeads™ human T cell activator products, Thermo Fisher Scientific, Waltham, Mass.)). The immunostimulatory cells, or target antigen sensitized immunostimulatory cells (for example, T cells), can optionally be expanded prior to or after ex vivo genetic engineering. Expansion of the cells is particularly useful to increase the number of cells for administration to a subject. Such methods for expansion of immune cells are well known in the art (see Kaiser et al., Cancer Gene Therapy 22:72-78 (2015); Wolfl et al., Nat. Protocols 9:950-966 (2014)). Furthermore, the cells can optionally be cryopreserved after isolation and/or genetic engineering, and/or expansion of genetically engineered cells (see Kaiser et al., supra, 2015)). Methods for cyropreserving cells are well known in the art (see, for example, Freshney, Culture of Animal Cells: A Manual of Basic Techniques, 4th ed., Wiley-Liss, New York (2000); Harrison and Rae, General Techniques of Cell Culture, Cambridge University Press (1997)).

In a preferred embodiment, a transgene as described herein includes the necessary elements providing for expression of the protein(s) encoded by the transgene in an immunostimulatory cell of the invention.

The invention also provides one or more transgenes as described above. In a specific embodiment, the transgene comprises the nucleotide sequences as described above. In another embodiment, the transgene consists essentially of the nucleotide sequences as described above. In another embodiment, the transgene consists of the nucleotide sequences as described above, plus any additional nucleotide sequences that may be necessary for the protein coding sequence(s) of the transgene to be expressible or expressed in the immunostimulatory cell. The invention further provides a vector comprising a transgene as described above. The invention further provides one or more vectors comprising one or more transgenes as described above. Preferably, the vectors are purified.

The one or more nucleotide sequences described above can be introduced into the immunostimulatory cell using one or more suitable expression vectors (i.e., transgenes), for example, by transduction. The nucleotide sequences can be on separate vectors or on the same vector, as desired. For example, a nucleotide sequence described herein can be cloned into a suitable vector, such as a retroviral vector, and introduced into the immunostimulatory cell using well known molecular biology techniques (see Ausubel et al., Current Protocols in Molecular Biology, John Wiley and Sons, Baltimore, Md. (1999)). Any vector suitable for expression in a cell of the invention, particularly a human immunostimulatory cell, can be employed. The vectors contain suitable expression elements such as promoters that provide for expression of the encoded nucleic acids in the immunostimulatory cell. In the case of a retroviral vector, cells can optionally be activated to increase transduction efficiency (see Parente-Pereira et al., J. Biol. Methods 1(2) e7 (doi 10.14440/jbm.2014.30) (2014); Movassagh et al., Hum. Gene Ther. 11:1189-1200 (2000); Rettig et al., Mol. Ther. 8:29-41 (2003); Agarwal et al., J. Virol. 72:3720-3728 (1998); Pollok et al., Hum. Gene Ther. 10:2221-2236 (1998); Quinn et al., Hum. Gene Ther. 9:1457-1467 (1998); see also commercially available methods such as Dynabeads™ human T cell activator products, Thermo Fisher Scientific, Waltham, Mass.).

In a specific embodiment, one or more nucleic acids are used to transduce both CD4⁺ and CD8⁺ T cells. In such an embodiment, administration of the transduced T cells to a subject should generate both helper and cytotoxic T lymphocyte (CTL) responses in the subject, resulting in a sustained anti-tumor or anti-infection response.

In one embodiment, the vector is a retroviral vector, for example, a gamma retroviral or lentiviral vector, which is employed for the introduction of one or more nucleotide sequences into the immunostimulatory cell. For genetic modification of the cells, a retroviral vector is generally employed for transduction. However, it is understood that any suitable viral vector or non-viral delivery system can be used. Combinations of a retroviral vector and an appropriate packaging line are also suitable, where the capsid proteins will be functional for infecting human cells. Various amphotropic virus-producing cell lines are known, including, but not limited to, PA12 (Miller et al., Mol. Cell. Biol. 5:431-437 (1985)); PA317 (Miller et al., Mol. Cell. Biol. 6:2895-2902(1986)); and CRIP (Danos et al., Proc. Natl. Acad. Sci. USA 85:6460-6464 (1988)). Non-amphotropic particles are suitable too, for example, particles pseudotyped with VSVG, RD114 or GALV envelope and any other known in the art (Relander et al., Mol. Therap. 11:452-459 (2005)). Possible methods of transduction also include direct co-culture of the cells with producer cells (for example, Bregni et al., Blood 80:1418-1422 (1992)), or culturing with viral supernatant alone or concentrated vector stocks with or without appropriate growth factors and polycations (see, for example, Xu et al., Exp. Hemat. 22:223-230 (1994); Hughes, et al. J. Clin. Invest. 89:1817-1824 (1992)).

Generally, the chosen vector exhibits high efficiency of infection and stable integration and expression (see, for example, Cayouette et al., Human Gene Therapy 8:423-430 (1997); Kido et al., Current Eye Research 15:833-844 (1996); Bloomer et al., J. Virol. 71:6641-6649 (1997); Naldini et al., Science 272:263 267 (1996); and Miyoshi et al., Proc. Natl. Acad. Sci. U.S.A. 94:10319-10323 (1997)). Other viral vectors that can be used include, for example, adenoviral, lentiviral, and adeno-associated viral vectors, vaccinia virus, a bovine papilloma virus derived vector, or a herpes virus, such as Epstein-Barr Virus (see, for example, Miller, Hum. Gene Ther. 1(1):5-14 (1990); Friedman, Science 244:1275-1281 (1989); Eglitis et al., BioTechniques 6:608-614 (1988); Tolstoshev et al., Current Opin. Biotechnol. 1:55-61 (1990); Sharp, Lancet 337:1277-1278 (1991); Cornetta et al., Prog. Nucleic Acid Res. Mol. Biol. 36:311-322 (1989); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S- 83S (1995)). Retroviral vectors are particularly well developed and have been used in clinical settings (Rosenberg et al., N. Engl. J. Med. 323:370 (1990); Anderson et al., U.S. Pat. No. 5,399,346).

Particularly useful vectors for use according to the invention include vectors that have been used in human gene therapy. In one non-limiting embodiment, a vector is a retroviral vector. The use of retroviral vectors for expression in T cells or other immune cells, including engineered CART cells, has been described (see Scholler et al., Sci. Transl. Med. 4:132-153 (2012; Parente-Pereira et al., J. Biol. Methods 1(2):e7 (1-9)(2014); Lamers et al., Blood 117(1):72-82 (2011); Reviere et al., Proc. Natl. Acad. Sci. USA 92:6733-6737 (1995)). In one embodiment, the vector is an SGF retroviral vector such as an SGF γ-retroviral vector, which is Moloney murine leukemia-based retroviral vector. SGF vectors have been described previously (see, for example, Wang et al., Gene Therapy 15:1454-1459 (2008)).

The vectors of the invention employ suitable promoters for expression in a particular host cell. The promoter can be an inducible promoter or a constitutive promoter. In a particular embodiment, the promoter of an expression vector provides expression in an immunostimulatory cell, such as a T cell. Non-viral vectors can be used as well, so long as the vector contains suitable expression elements for expression in the immunostimulatory cell. Some vectors, such as retroviral vectors, can integrate into the host genome (thus, such vectors can be used to integrate the nucleotide sequences or transgenes described herein into the genome of the immunostimulatory cells described above). If desired, targeted integration can be implemented using technologies such as a nuclease, transcription activator-like effector nucleases (TALENs), Zinc-finger nucleases (ZFNs), and/or clustered regularly interspaced short palindromic repeats (CRISPRs), by homologous recombination, and the like (Gersbach et al., Nucl. Acids Res. 39:7868-7878 (2011); Vasileva, et al. Cell Death Dis. 6:e1831. (Jul. 23, 2015); Sontheimer, Hum. Gene Ther. 26(7):413-424 (2015)).

The vectors and constructs can optionally be designed to include a reporter. For example, the vector can be designed to express a reporter protein, which can be useful to identify cells comprising the vector or nucleic acids provided on the vector, such as nucleic acids that have integrated into the host chromosome. In one embodiment, the reporter can be expressed from a bicistronic or multicistronic expression construct with the CAR, dominant negative form, immunomodulatory agent, and/or IL-12. Exemplary reporter proteins include, but are not limited to, fluorescent proteins, such as mCherry, green fluorescent protein (GFP), blue fluorescent protein, for example, EBFP, EBFP2, Azurite, and mKalamal, cyan fluorescent protein, for example, ECFP, Cerulean, and CyPet, and yellow fluorescent protein, for example, YFP, Citrine, Venus, and YPet. In an additional embodiment, a vector construct can comprise a P2A sequence, which provides for optional co-expression of a reporter molecule. P2A is a self-cleaving peptide sequence, which can be used for bicistronic or multicistronic expression of protein sequences (see Szymczak et al., Expert Opin. Biol. Therapy 5(5):627-638 (2005)).

Assays can be used to determine the transduction efficiency using routine molecular biology techniques. If a marker has been included in the construct, such as a fluorescent protein, gene transfer efficiency can be monitored by FACS analysis to quantify the fraction of transduced (for example, GFP⁺) immunostimulatory cells, such as T cells, and/or by quantitative PCR. Using a well-established cocultivation system (Gade et al., Cancer Res. 65:9080-9088 (2005); Gong et al., Neoplasia 1:123-127 (1999); Latouche et al., Nat. Biotechnol. 18:405-409 (2000)) it can be determined whether fibroblast AAPCs expressing cancer antigen (vs. controls) direct cytokine release from transduced immunostimulatory cells, such as T cells, expressing a CAR (cell supernatant LUMINEX (Austin TX) assay for IL-2, IL-4, IL-10, IFN-γ, TNF-α, and GM-CSF), T cell proliferation (by carboxyfluorescein succinimidyl ester (CF SE) labeling), and T cell survival (by Annexin V staining). The influence of CD80 and/or 4-1BBL on T cell survival, proliferation, and efficacy can be evaluated. T cells can be exposed to repeated stimulation by target antigen positive target cells, and it can be determined whether T cell proliferation and cytokine response remain similar or diminished with repeated stimulation. The target antigen CAR constructs can be compared side by side under equivalent assay conditions. Cytotoxicity assays with multiple E:T ratios can be conducted using chromium-release assays.

In addition to providing a nucleic acid encoding a polypeptide in a vector for expression in an immunostimulatory cell, a nucleic acid encoding the polypeptide can also be provided in other types of vectors more suitable for genetic manipulation, such as for expression of various constructs in a bacterial cell such as E. coli. Such vectors can be any of the well known expression vectors, including commercially available expression vectors (see in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, Md. (1999).

If desired, a nucleic acid encoding a polypeptide for genetic engineering of a cell of the invention, can be codon optimized to increase efficiency of expression in an immunostimulatory cell thereof. Codon optimization can be used to achieve higher levels of expression in a given cell. Factors that are involved in different stages of protein expression include codon adaptability, mRNA structure, and various cis-elements in transcription and translation. Any suitable codon optimization methods or technologies that are known to one skilled in the art can be used to modify the polynucleotides encoding the polypeptides. Such codon optimization methods are well known, including commercially available codon optimization services, for example, OptimumGene™ (GenScript; Piscataway, N.J.), Encor optimization (EnCor Biotechnology; Gainseville Fla.), Blue Heron (Blue Heron Biotech; Bothell, Wash.), and the like. Optionally, multiple codon optimizations can be performed based on different algorithms, and the optimization results blended to generate a codon optimized nucleic acid encoding a polypeptide.

Further modification can be introduced to the immunostimulatory cells of the invention. For example, the cells can be modified to address immunological complications and/or targeting by the CAR to healthy tissues that express the same target antigens as the tumor cells or infected cells. For example, a suicide gene can be introduced into the cells to provide for depletion of the cells when desired. In a specific embodiment, the immunostimulatory cell further recombinantly expresses a suicide gene. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In a specific embodiment, the suicide gene comprises inducible Caspase 9. Agents are administered to the subject to which the cells containing the suicide genes have been administered, including but not limited to, gancilovir (GCV) for hsv-tk (Greco et al., Frontiers Pharmacol. 6:95 (2015); Barese et al., Mol. Therapy 20:1932-1943 (2012)), AP1903 for iCasp-9 (Di Stasi et al., N. Engl. J. Med. 365:1673-1683 (2011), and cetuximab for EGFRt (U.S. Pat. No. 8,802,374), to promote cell death. In one embodiment, administration of a prodrug designed to activate the suicide gene, for example, a prodrug such as AP1903 that can activate iCasp-9, triggers apoptosis in the suicide gene-activated cells. In one embodiment, iCasp9 consists of the sequence of the human FK506-binding protein (FKBP12; GenBank number, AH002818 (AH002818.1, M92422.1, GI:182645; AH002818.2, GI:1036032368)) with an F36V mutation, connected through a Ser-Gly-Gly-Gly-Ser linker (SEQ ID NO:48) to the gene encoding human caspase 9 (CASP9; GenBank number, NM_001229 (NM 001229.4, GI:493798577)), which has had its endogenous caspase activation and recruitment domain deleted. FKBP12-F36V binds with high affinity to an otherwise bioinert small-molecule dimerizing agent, AP1903. In the presence of AP1903, the iCasp9 promolecule dimerizes and activates the intrinsic apoptotic pathway, leading to cell death (Di Stasi et al., N. Engl. J. Med. 365:1673-1683 (2011)). In another embodiment, the suicide gene is an EGFRt polypeptide. The EGFRt polypeptide can provide for cell elimination by administering anti-EGFR monoclonal antibody, for example, cetuximab. The suicide gene can be expressed on a separate vector or, optionally, expressed within the vector encoding one or more nucleotide sequences described herein, and can be a bicistronic or multicistronic construct joined to one or more nucleotide sequences described herein.

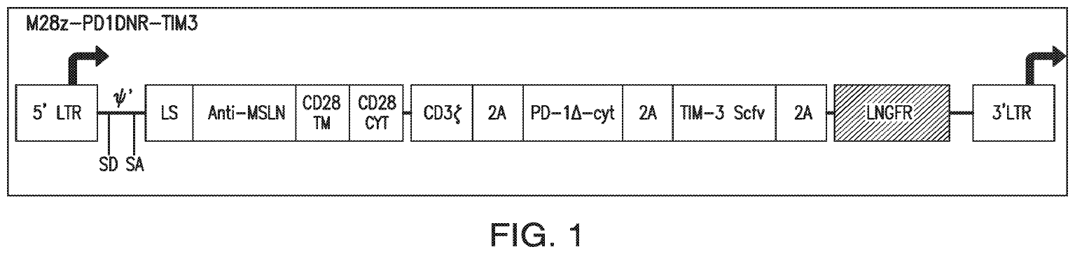

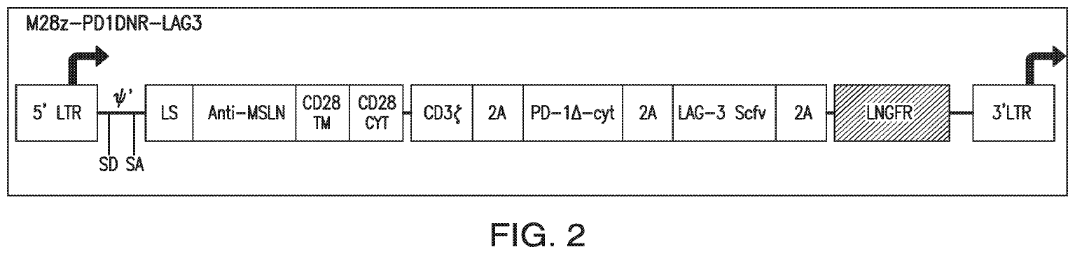

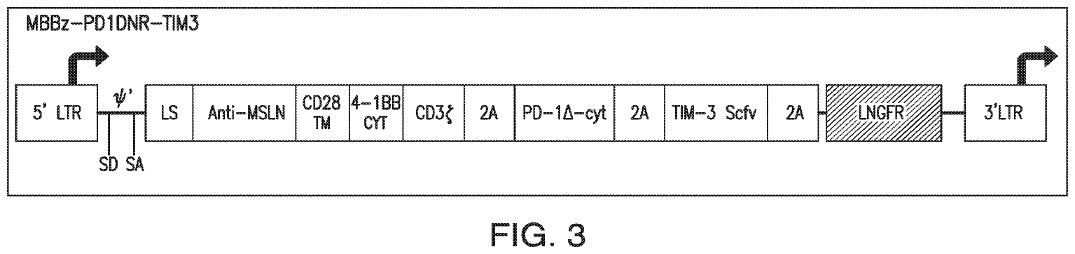

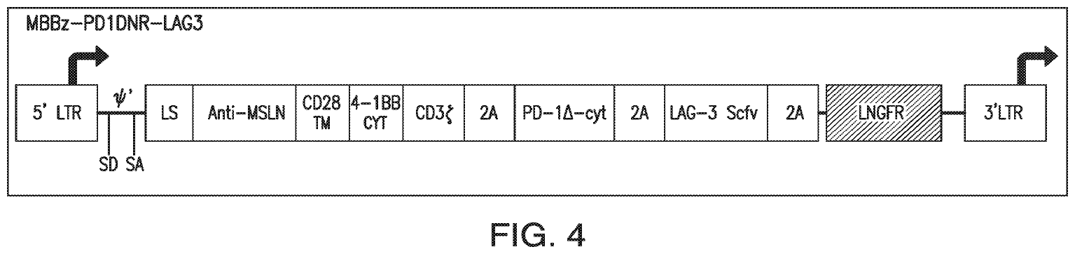

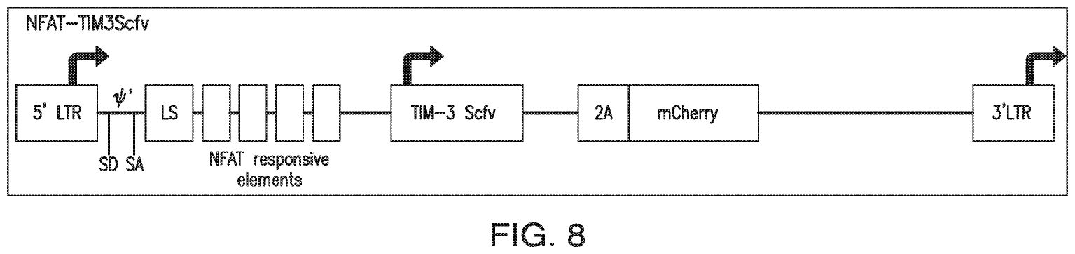

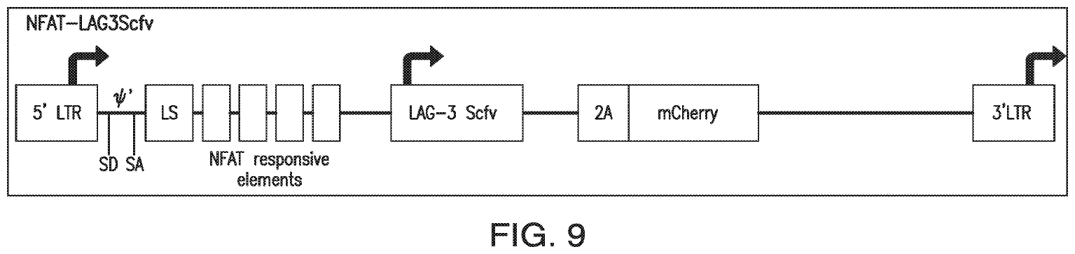

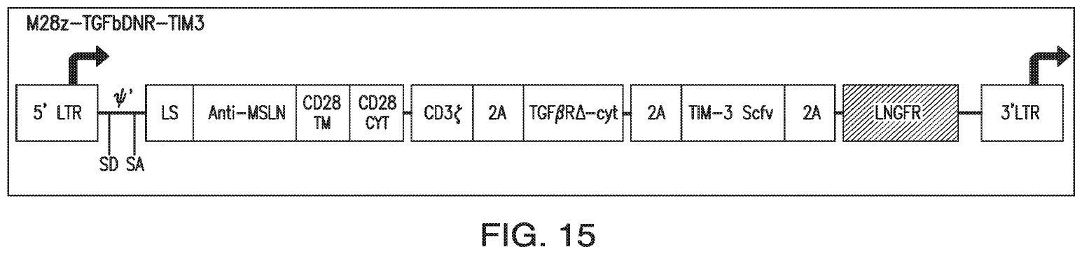

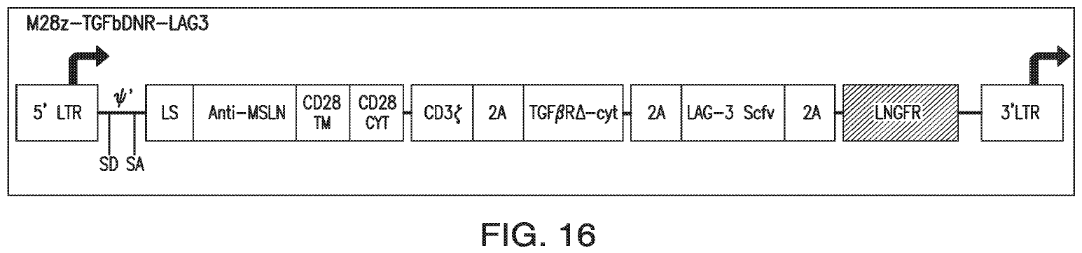

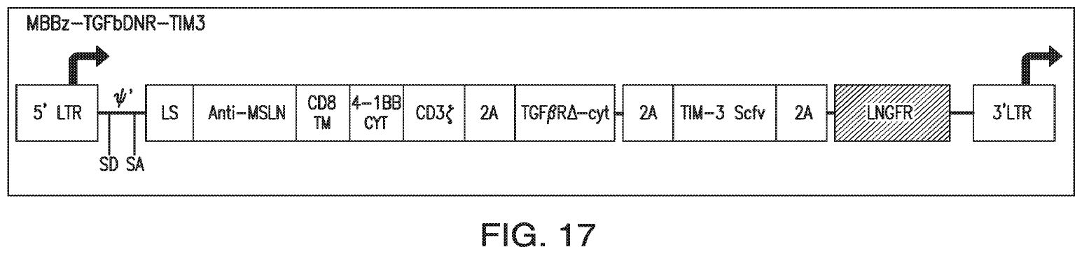

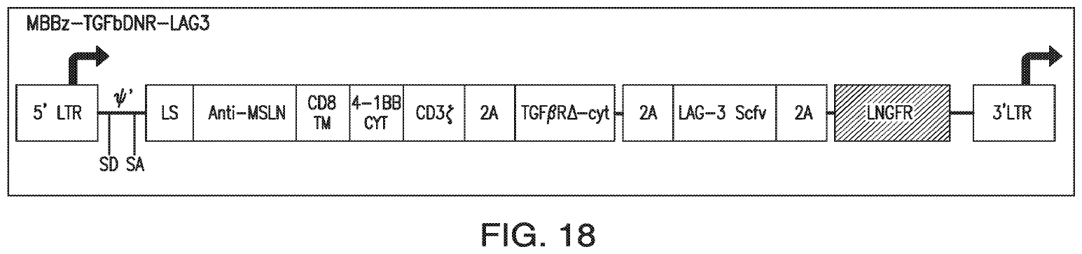

In a specific embodiment of the invention, the immunostimulatory cell comprises a transgene(s) as illustrated in any of the FIGS. 1-18.

In a specific embodiment, the invention provides an immunostimulatory cell as described herein, wherein the cancer antigen is mesothelin and the inhibitor of a cell-mediated immune response is PD-1.

In a specific embodiment, the invention provides an immunostimulatory cell as described herein, wherein the cancer antigen is mesothelin, the inhibitor of a cell-mediated immune response is PD-1, and the immune checkpoint inhibitor to which the immunomodulatory agent binds is TIM-3.

In a specific embodiment, the invention provides an immunostimulatory cell as described herein, wherein the cancer antigen is mesothelin, the inhibitor of a cell-mediated immune response is PD-1, and the immune checkpoint inhibitor to which the immunomodulatory agent binds is LAG-3.

In a specific embodiment, the invention provides an immunostimulatory cell as described herein, wherein the cancer antigen is mesothelin and the inhibitor of a cell-mediated immune response is transforming growth factor β (TGF-β) receptor.

In a specific embodiment, the invention provides an immunostimulatory cell as described herein, wherein the cancer antigen is mesothelin, the inhibitor of a cell-mediated immune response is TGF-β receptor, and the immune checkpoint inhibitor to which the immunomodulatory agent binds is TIM-3.

In a specific embodiment, the invention provides an immunostimulatory cell as described herein, wherein the cancer antigen is mesothelin, the inhibitor of a cell-mediated immune response is TGF-β receptor, and the immune checkpoint inhibitor to which the immunomodulatory agent binds is LAG-3.

The invention provides immunostimulatory cells comprising the nucleotide sequence(s), transgene(s), construct(s), or vector(s) described herein. In specific embodiments of the invention, the immunostimulatory cells express the transgene(s) or protein(s) encoded by the nucleotide sequence(s) described herein.

### 7.2 Chimeric Antigen Receptors (CARs)

The CAR that is recombinantly expressed by a cell of the invention has an antigen binding domain that binds to a target antigen associated with a mammalian disease or disorder (i.e., a cancer or an infection with a pathogen). In specific embodiments, the CAR can be a “first generation,” “second generation” or “third generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. 21(2):215-223 (2009); Hollyman et al., J. Immunother. 32:169-180 (2009)).

“First generation” CARs are typically composed of an extracellular antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3ζ-chain, which is the primary transmitter of signals from endogenous T cell receptors (TCRs). “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second-generation” CARs for use in the invention comprise a target antigen-binding domain fused to an intracellular signaling domain capable of activating immune cells such as T cells and a co-stimulatory domain designed to augment immune cell, such as T cell, potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell. “Second generation” CARs provide both co-stimulation, for example, by CD28 or 4-1BB domains, and activation, for example, by a CD3 signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)). “Third generation” CARs provide multiple co-stimulation, for example, by comprising both CD28 and 4-1BB domains, and activation, for example, by comprising a CD3ζ activation domain.

In the embodiments disclosed herein, the CARs generally comprise an extracellular antigen binding domain, a transmembrane domain and an intracellular domain, as described above, where the extracellular antigen binding domain binds to a target antigen. In a particular non-limiting embodiment, the extracellular antigen-binding domain is an scFv.

The extracellular antigen-binding domain of a CAR is usually derived from a monoclonal antibody (mAb) or from receptors or their ligands.

The design of CARs is well known in the art (see, for example, reviews by Sadelain et al., Cancer Discov. 3(4):388-398 (2013); Jensen et al., Immunol. Rev. 257:127-133 (2014); Sharpe et al., Dis. Model Mech. 8(4):337-350 (2015), and references cited therein). A CAR directed to a desired target antigen can be generated using well known methods for designing a CAR, including those as described herein. A CAR, whether a first, second or third generation CAR, can be readily designed by fusing a target antigen binding activity, for example, an scFv antibody directed to the target antigen, to an immune cell signaling domain, such as a T cell receptor cytoplasmic/intracellular domain. As described above, the CAR generally has the structure of a cell surface receptor, with the target antigen binding activity, such as an scFv, as at least a portion of the extracellular domain, fused to a transmembrane domain, which is fused to an intracellular domain that has cell signaling activity in an immunostimulatory cell, such as a T cell. The target antigen CAR can include co-stimulatory molecules, as described herein. One skilled in the art can readily select appropriate transmembrane domains, as described herein and known in the art, and intracellular domains to provide the desired signaling capability in the immunostimulatory cell, such as a T cell.

A CAR for use in the present invention comprises an extracellular domain that includes an antigen binding domain that binds to a target antigen. The antigen binding domain binds to an antigen on the target cancer or infected cell or tissue. Such an antigen binding domain is generally derived from an antibody. In one embodiment, the antigen binding domain can be an scFv or a Fab, or any suitable antigen binding fragment of an antibody (see Sadelain et al., Cancer Discov. 3:388-398 (2013)). Many antibodies or antigen binding domains derived from antibodies that bind to a target antigen are known in the art. Alternatively, such antibodies or antigen binding domains can be produced by routine methods. Methods of generating an antibody are well known in the art, including methods of producing a monoclonal antibody or screening a library to obtain an antigen binding polypeptide, including screening a library of human Fabs (Winter and Harris, Immunol. Today 14:243-246 (1993); Ward et al., Nature 341:544-546 (1989); Harlow and Lane, Antibodies: A Laboratory Manual, Cold Spring Harbor Laboratory Press (1988); Hilyard et al., Protein Engineering: A practical approach (IRL Press 1992); Borrabeck, Antibody Engineering, 2nd ed. (Oxford University Press 1995); Huse et al., Science 246:1275-1281 (1989)). For the CAR, the antigen binding domain derived from an antibody can be human, humanized, chimeric, CDR-grafted, and the like, as desired. For example, if a mouse monoclonal antibody is a source antibody for generating the antigen binding domain of a CAR, such an antibody can be humanized by grafting CDRs of the mouse antibody onto a human framework (see Borrabeck, supra, 1995), which can be beneficial for administering the CAR to a human subject. In a preferred embodiment, the antigen binding domain is an scFv. The generation of scFvs is well known in the art (see, for example, Huston, et al., Proc. Nat. Acad. Sci. USA 85:5879-5883 (1988); Ahmad et al.,Clin. Dev. Immunol. 2012: ID980250 (2012); U.S. Pat. Nos. 5,091,513, 5,132,405 and 4,956,778; and U.S. Patent Publication Nos. 20050196754 and 20050196754)).

With respect to obtaining a target antigen binding activity, one skilled in the art can readily obtain a suitable target antigen binding activity, such as an antibody, using any of the well known methods for generating and screening for an antibody that binds to a desired antigen, as disclosed herein, including the generation of an scFv that binds to a target antigen, which is particularly useful in a CAR. In addition, a number target antigen antibodies, in particular monoclonal antibodies, are commercially available and can also be used as a source for a target antigen binding activity, such as an scFv, to generate a CAR.

Alternative to using an antigen binding domain derived from an antibody, a CAR extracellular domain can comprise a ligand or extracellular ligand binding domain of a receptor (see Sadelain et al., Cancer Discov. 3:388-398 (2013); Sharpe et al., Dis. Model Mech. 8:337-350 (2015)). In this case, the ligand or extracellular ligand binding domain of a receptor provides to the CAR the ability to target the cell expressing the CAR to the corresponding receptor or ligand. The ligand or extracellular ligand binding domain is selected such that the cell expressing the CAR is targeted to a cancer cell, tumor, or infected cell (see Sadelain et al., Cancer Discov. 3:388-398 (2013); Sharpe et al., Dis. Model Mech. 8:337-350 (2015), and references cited therein). In an embodiment of the invention, the ligand or extracellular ligand binding domain is selected to bind to a target antigen that is the corresponding receptor or ligand (see Sadelain et al, Cancer Discov. 3:388-398 (2013)).

As described above, a CAR also contains a signaling domain that functions in the immunostimulatory cell, expressing the CAR. Such a signaling domain can be, for example, derived from CDC or Fc receptor y (see Sadelain et al., Cancer Discov. 3:388-398 (2013)). In general, the signaling domain will induce persistence, trafficking and/or effector functions in the transduced immunostimulatory cells such as T cells (Sharpe et al., Dis. Model Mech. 8:337-350 (2015); Finney et al., J. Immunol. 161:2791-2797 (1998); Krause et al., J. Exp. Med. 188:619-626 (1998)). In the case of CDζ or Fc receptor γ, the signaling domain corresponds to the intracellular domain of the respective polypeptides, or a fragment of the intracellular domain that is sufficient for signaling. Exemplary signaling domains are described below in more detail.

Exemplary polypeptides are described herein with reference to GenBank numbers, GI numbers and/or SEQ ID NOS. It is understood that one skilled in the art can readily identify homologous sequences by reference to sequence sources, including but not limited to GenBank (ncbi.nlm.nih.gov/genbank/) and EMBL (embl.org/).

CD3ζ. In a non-limiting embodiment, a CAR can comprise a signaling domain derived from a CD3ζ polypeptide, for example, a signaling domain derived from the intracellular domain of CD3ζ, which can activate or stimulate an immune cell, for example, a T cell, or precursor cell thereof. CD3ζ comprises 3 Immune-receptor-Tyrosine-based-Activation-Motifs (ITAMs), and transmits an activation signal to the cell, for example, a cell of the lymphoid lineage such as a T cell, after antigen is bound. A CD3ζ polypeptide can have an amino acid sequence corresponding to the sequence having GenBank No. NP_932170 (NP_932170.1, GI:37595565; see below), or fragments thereof. In one embodiment, the CD3ζ polypeptide has an amino acid sequence of amino acids 52 to 164 of the CD3ζ polypeptide sequence provided below, or a fragment thereof that is sufficient for signaling activity. An exemplary CAR is Mz, which has an intracellular domain comprising a CD3ζ polypeptide comprising amino acids 52 to 164 of the CD3ζ polypeptide sequence provided below. Another exemplary CAR is M28z, which has an intracellular domain comprising a CD3ζ polypeptide comprising amino acids 52 to 164 of the CD3ζ polypeptide provided below. Still another exemplary CAR is MBBz, which has an intracellular domain comprising a CD3ζ polypeptide comprising amino acids 52 to 164 of the CD3ζ polypeptide provided below. Yet another exemplary CAR is P28z, which has an intracellular domain derived from a CD3ζ polypeptide. See GenBank NP_932170 for reference to domains within CD3ζ, for example, signal peptide, amino acids 1 to 21; extracellular domain, amino acids 22 to 30; transmembrane domain, amino acids 31 to 51; intracellular domain, amino acids 52 to 164.

| (NP_932170; SEQ ID NO: 1) | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

It is understood that a “CD3ζ nucleic acid molecule” refers to a polynucleotide encoding a CD3ζ polypeptide. In one embodiment, the CD3ζ nucleic acid molecule encoding the CD3ζ polypeptide comprised in the intracellular domain of a CAR, including exemplary CARs Mz, M28z, or MBBz, comprises a nucleotide sequence as set forth below.

(SEQ ID NO: 2)

AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGCTAA

In certain non-limiting embodiments, an intracellular domain of a CAR can further comprise at least one co-stimulatory signaling domain. Such a co-stimulatory signaling domain can provide increased activation of an immunostimulatory cell. A co-stimulatory signaling domain can be derived from a CD28 polypeptide, a 4-1BB polypeptide, an OX40 polypeptide, an ICOS polypeptide, a DAP10 polypeptide, a 2B4 polypeptide, and the like. CARs comprising an intracellular domain that comprises a co-stimulatory signaling region comprising 4-1BB, ICOS or DAP-10 have been described previously (see U.S. 7,446,190, which is incorporated herein by reference, which also describes representative sequences for 4-1BB, ICOS and DAP-10). In some embodiments, the intracellular domain of a CAR can comprise a co-stimulatory signaling region that comprises two co-stimulatory molecules, such as CD28 and 4-1BB (see Sadelain et al., Cancer Discov. 3(4):388-398 (2013)), or CD28 and OX40, or other combinations of co-stimulatory ligands, as disclosed herein.

CD28. Cluster of Differentiation 28 (CD28) is a protein expressed on T cells that provides co-stimulatory signals for T cell activation and survival. CD28 is the receptor for CD80 (B7.1) and CD86 (B7.2) proteins. In one embodiment, a CAR can comprise a co-stimulatory signaling domain derived from CD28. For example, as disclosed herein, a CAR can include at least a portion of an intracellular/cytoplasmic domain of CD28, for example an intracellular/cytoplasmic domain that can function as a co-stimulatory signaling domain. A CD28 polypeptide can have an amino acid sequence corresponding to the sequence having GenBank No. P10747 (P10747.1, GI:115973) or NP_006130 (NP_006130.1, GI:5453611), as provided below, or fragments thereof If desired, CD28 sequences additional to the intracellular domain can be included in a CAR of the invention. For example, a CAR can comprise the transmembrane of a CD28 polypeptide. In one embodiment, a CAR can have an amino acid sequence comprising the intracellular domain of CD28 corresponding to amino acids 180 to 220 of CD28, or a fragment thereof. In another embodiment, a CAR can have an amino acid sequence comprising the transmembrane domain of CD28 corresponding to amino acids 153 to 179, or a fragment thereof. M28z is an exemplary CAR, which comprises a co-stimulatory signaling domain corresponding to an intracellular domain of CD28. M28z also comprises a transmembrane domain derived from CD28. Thus, M28z exemplifies a CAR that comprises two domains from CD28, a co-stimulatory signaling domain and a transmembrane domain. In one embodiment, a CAR has an amino acid sequence comprising the transmembrane domain and the intracellular domain of CD28 and comprises amino acids 153 to 220 of CD28. In another embodiment, a CAR is exemplified by M28z CAR and comprises amino acids 117 to 220 of CD28. Another exemplary CAR having a transmembrane domain and intracellular domain of CD28 is P28z. In one embodiment, a CAR can comprise a transmembrane domain derived from a CD28 polypeptide comprising amino acids 153 to 179 of the CD28 polypeptide provided below. See GenBank NP_006130 for reference to domains within CD28, for example, signal peptide, amino acids 1 to 18; extracellular domain, amino acids 19 to 152; transmembrane domain, amino acids 153 to 179; intracellular domain, amino acids 180 to 220. It is understood that sequences of CD28 that are shorter or longer than a specific delineated domain can be included in a CAR, if desired.

| (NP_006130; SEQ ID NO: 3) | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

It is understood that a “CD28 nucleic acid molecule” refers to a polynucleotide encoding a CD28 polypeptide. In one embodiment, the CD28 nucleic acid molecule encoding the CD28 polypeptide of M28z comprising the transmembrane domain and the intracellular domain, for example, the co-stimulatory signaling region, comprises a nucleotide sequence as set forth below.

(SEQ ID NO: 4)

ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAG
GAGTAAGAGGAGCAGGCTCCTGCACAGTGACTACATGAACATGACTCCCC
GCCGCCCCGGGCCCACCCGCAAGCATTACCAGCCCTATGCCCCACCACGC
GACTTCGCAGCCTATCGCTCC

4-1BB. 4-1BB, also referred to as tumor necrosis factor receptor superfamily member 9, can act as a tumor necrosis factor (TNF) ligand and have stimulatory activity. In one embodiment, a CAR can comprise a co-stimulatory signaling domain derived from 4-1BB. A 4-1BB polypeptide can have an amino acid sequence corresponding to the sequence having GenBank No. P41273 (P41273.1, GI:728739) or NP_001552 (NP_001552.2, GI:5730095) or fragments thereof. In one embodiment, a CAR can have a co-stimulatory domain comprising the intracellular domain of 4-1BB corresponding to amino acids 214 to 255, or a fragment thereof. In another embodiment, a CAR can have a transmembrane domain of 4-1BB corresponding to amino acids 187 to 213, or a fragment thereof. An exemplary CAR is MBBz, which has an intracellular domain comprising a 4-1BB polypeptide (for example, amino acids 214 to 255 of NP_001552, SEQ ID NO:5). See GenBank NP_001552 for reference to domains within 4-1BB, for example, signal peptide, amino acids 1 to 17; extracellular domain, amino acids 18 to 186; transmembrane domain, amino acids 187 to 213; intracellular domain, amino acids 214 to 255. It is understood that sequences of 4-1BB that are shorter or longer than a specific delineated domain can be included in a CAR, if desired. It is also understood that a “4-1BB nucleic acid molecule” refers to a polynucleotide encoding a 4-1BB polypeptide.

| (NP_001552; SEQ ID NO: 5) | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

OX40. OX40, also referred to as tumor necrosis factor receptor superfamily member 4 precursor or CD134, is a member of the TNFR-superfamily of receptors. In one embodiment, a CAR can comprise a co-stimulatory signaling domain derived from OX40. An OX40 polypeptide can have an amino acid sequence corresponding to the sequence having GenBank No. P43489 (P43489.1, GI:1171933) or NP_003318 (NP_003318.1, GI:4507579), provided below, or fragments thereof In one embodiment, a CAR can have a co-stimulatory domain comprising the intracellular domain of OX40 corresponding to amino acids 236 to 277, or a fragment thereof. In another embodiment, a CAR can have an amino acid sequence comprising the transmembrane domain of OX40 corresponding to amino acids 215 to 235 of OX40, or a fragment thereof. See GenBank NP_003318 for reference to domains within OX40, for example, signal peptide, amino acids 1 to 28; extracellular domain, amino acids 29 to 214; transmembrane domain, amino acids 215 to 235; intracellular domain, amino acids 236 to 277. It is understood that sequences of OX40 that are shorter or longer than a specific delineated domain can be included in a CAR, if desired. It is also understood that an “0×40 nucleic acid molecule” refers to a polynucleotide encoding an 0×40 polypeptide.

| (NP_003318; SEQ ID NO: 6) | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

ICOS. Inducible T-cell costimulator precursor (ICOS), also referred to as CD278, is a CD28-superfamily costimulatory molecule that is expressed on activated T cells. In one embodiment, a CAR can comprise a co-stimulatory signaling domain derived from ICOS. An ICOS polypeptide can have an amino acid sequence corresponding to the sequence having GenBank No. NP_036224 (NP_036224.1, GI:15029518), provided below, or fragments thereof. In one embodiment, a CAR can have a co-stimulatory domain comprising the intracellular domain of ICOS corresponding to amino acids 162 to 199 of ICOS. In another embodiment, a CAR can have an amino acid sequence comprising the transmembrane domain of ICOS corresponding to amino acids 141 to 161 of ICOS, or a fragment thereof. See GenBank NP_036224 for reference to domains within ICOS, for example, signal peptide, amino acids 1 to 20; extracellular domain, amino acids 21 to 140; transmembrane domain, amino acids 141 to 161; intracellular domain, amino acids 162 to 199. It is understood that sequences of ICOS that are shorter or longer than a specific delineated domain can be included in a CAR, if desired. It is also understood that an “ICOS nucleic acid molecule” refers to a polynucleotide encoding an ICOS polypeptide.

| (NP_036224; SEQ ID NO: 7) | |
|---|---|
| 1 | MKSGLWYFFL FCLRIKVLTG EINGSANYEM FIFHNGGVQI LCKYPDIVQQ FKMQLLKGGQ |
| 61 | ILCDLTKTKG SGNTVSIKSL KFCHSQLSNN SVSFFLYNLD HSHANYYFCN LSIFDPPPFK |
| 121 | VTLTGGYLHI YESQLCCQLK FWLPIGCAAF VVVCILGCIL ICWLTKKKYS SSVHDPNGEY |
| 181 | MFMRAVNTAK KSRLTDVTL |

DAP10. DAP10, also referred to as hematopoietic cell signal transducer, is a signaling subunit that associates with a large family of receptors in hematopoietic cells. In one embodiment, a CAR can comprise a co-stimulatory domain derived from DAP10. A DAP10 polypeptide can have an amino acid sequence corresponding to the sequence having GenBank No. NP_055081.1 (GI:15826850), provided below, or fragments thereof. In one embodiment, a CAR can have a co-stimulatory domain comprising the intracellular domain of DAP10 corresponding to amino acids 70 to 93, or a fragment thereof. In another embodiment, a CAR can have a transmembrane domain of DAP10 corresponding to amino acids 49 to 69, or a fragment thereof. See GenBank NP_055081.1 for reference to domains within DAP10, for example, signal peptide, amino acids 1 to 19; extracellular domain, amino acids 20 to 48; transmembrane domain, amino acids 49 to 69; intracellular domain, amino acids 70 to 93. It is understood that sequences of DAP10 that are shorter or longer than a specific delineated domain can be included in a CAR, if desired. It is also understood that a “DAP10 nucleic acid molecule” refers to a polynucleotide encoding an DAP10 polypeptide.

| (NP_055081.1; SEQ ID NO: 8) | |
|---|---|
| 1 | MIHLGHILFL LLLPVAAAQT TPGERSSLPA FYPGTSGSCS GCGSLSLPLL AGLVAADAVA |
| 61 | SLLIVGAVFL CARPRRSPAQ EDGKVYINMP GRG |

The extracellular domain of a CAR can be fused to a leader or a signal peptide that directs the nascent protein into the endoplasmic reticulum and subsequent translocation to the cell surface. It is understood that, once a polypeptide containing a signal peptide is expressed at the cell surface, the signal peptide has generally been proteolytically removed during processing of the polypeptide in the endoplasmic reticulum and translocation to the cell surface. Thus, a polypeptide such as a CAR is generally expressed at the cell surface as a mature protein lacking the signal peptide, whereas the precursor form of the polypeptide includes the signal peptide. A signal peptide or leader can be essential if a CAR is to be glycosylated and/or anchored in the cell membrane. The signal sequence or leader is a peptide sequence generally present at the N-terminus of newly synthesized proteins that directs their entry into the secretory pathway. The signal peptide is covalently joined to the N-terminus of the extracellular antigen-binding domain of a CAR as a fusion protein. In one embodiment, the signal peptide comprises a CD8 polypeptide comprising amino acids MALPVTALLLPLALLLHAARP (SEQ ID NO:9). It is understood that use of a CD8 signal peptide is exemplary. Any suitable signal peptide, as are well known in the art, can be applied to a CAR to provide cell surface expression in an immunostimulatory cell (see Gierasch Biochem. 28:923-930 (1989); von Heijne, J. Mol. Biol. 184 (1):99-105 (1985)). Particularly useful signal peptides can be derived from cell surface proteins naturally expressed in the immunostimulatory cell, including any of the signal peptides of the polypeptides disclosed herein. Thus, any suitable signal peptide can be utilized to direct a CAR to be expressed at the cell surface of an immunostimulatory cell.

In certain non-limiting embodiments, an extracellular antigen-binding domain of a CAR can comprise a linker sequence or peptide linker connecting the heavy chain variable region and light chain variable region of the extracellular antigen-binding domain. In one non-limiting example, the linker comprises amino acids having the sequence set forth in GGGGSGGGGSGGGGS (SEQ ID NO:10).

In certain non-limiting embodiments, a CAR can also comprise a spacer region or sequence that links the domains of the CAR to each other. For example, a spacer can be included between a signal peptide and an antigen binding domain, between the antigen binding domain and the transmembrane domain, between the transmembrane domain and the intracellular domain, and/or between domains within the intracellular domain, for example, between a stimulatory domain and a co-stimulatory domain. The spacer region can be flexible enough to allow interactions of various domains with other polypeptides, for example, to allow the antigen binding domain to have flexibility in orientation in order to facilitate antigen recognition. The spacer region can be, for example, the hinge region from an IgG, the CH₂CH₃ (constant) region of an immunoglobulin, and/or portions of CD3 (cluster of differentiation 3) or some other sequence suitable as a spacer.

The transmembrane domain of a CAR generally comprises a hydrophobic alpha helix that spans at least a portion of the membrane. Different transmembrane domains result in different receptor stability. After antigen recognition, receptors cluster and a signal is transmitted to the cell. In an embodiment, the transmembrane domain of a CAR can be derived from another polypeptide that is naturally expressed in the immunostimulatory cell. In one embodiment, a CAR can have a transmembrane domain derived from CD8, CD28, CD3, CD4, 4-1BB, OX40, ICOS, CTLA-4, PD-1, LAG-3, 2B4, BTLA, or other polypeptides expressed in the immunostimulatory cell having a transmembrane domain, including others as disclosed herein. Optionally, the transmembrane domain can be derived from a polypeptide that is not naturally expressed in the immunostimulatory cell, so long as the transmembrane domain can function in transducing signal from antigen bound to the CAR to the intracellular signaling and/or co-stimulatory domains. It is understood that the portion of the polypeptide that comprises a transmembrane domain of the polypeptide can include additional sequences from the polypeptide, for example, additional sequences adjacent on the N-terminal or C-terminal end of the transmembrane domain, or other regions of the polypeptide, as desired.

CD8. Cluster of differentiation 8 (CD8) is a transmembrane glycoprotein that serves as a co-receptor for the T cell receptor (TCR). CD8 binds to a major histocompatibility complex (MHC) molecule and is specific for the class I MHC protein. In one embodiment, a CAR can comprise a transmembrane domain derived from CD8. A CD8 polypeptide can have an amino acid sequence corresponding to the sequence having GenBank No. NP_001139345.1 (GI:225007536), as provided below, or fragments thereof. In one embodiment, a CAR can have an amino acid sequence comprising the transmembrane domain of CD8 corresponding to amino acids 183 to 203, or fragments thereof In one embodiment, an exemplary CAR is Mz, which has a transmembrane domain derived from a CD8 polypeptide. In another embodiment, an exemplary CAR is MBBz, which has a transmembrane domain derived from a CD8 polypeptide. In one non-limiting embodiment, a CAR can comprise a transmembrane domain derived from a CD8 polypeptide comprising amino acids 183 to 203. In addition, a CAR can comprise a hinge domain comprising amino acids 137-182 of the CD8 polypeptide provided below. In another embodiment, a CAR can comprise amino acids 137-203 of the CD8 polypeptide provided below. In yet another embodiment, a CAR can comprise amino acids 137 to 209 of the CD8 polypeptide provided below. See GenBank NP_001139345.1 for reference to domains within CD8, for example, signal peptide, amino acids 1 to 21; extracellular domain, amino acids 22 to 182; transmembrane domain amino acids, 183 to 203; intracellular domain, amino acids 204 to 235. It is understood that additional sequence of CD8 beyond the transmembrane domain of amino acids 183 to 203 can be included in a CAR, if desired. It is further understood that sequences of CD8 that are shorter or longer than a specific dilineated domain can be included in a CAR, if desired. It also is understood that a “CD8 nucleic acid molecule” refers to a polynucleotide encoding a CD8 polypeptide.

| (NP_001139345.1; SEQ ID NO: 11) | |
|---|---|
| 1 | MALPVTALLL PLALLLHAAR PSQFRVSPLD RTWNLGETVE LKCQVLLSNP TSGCSWLFQP |
| 61 | RGAAASPTFL LYLSQNKPKA AEGLDTQRFS GKRLGDTFVL TLSDFRRENE GYYFCSALSN |
| 121 | SIMYFSHFVP VFLPAKPTTT PAPRPPTPAP TIASQPLSLR PEACRPAAGG AVHTRGLDFA |
| 181 | CDIYIWAPLA GTCGVLLLSL VITLYCNHRN RRRVCKCPRP VVKSGDKPSL SARYV |

CD4. Cluster of differentiation 4 (CD4), also referred to as T-cell surface glycoprotein CD4, is a glycoprotein found on the surface of immune cells such as T helper cells, monocytes, macrophages, and dendritic cells. In one embodiment, a CAR can comprise a transmembrane domain derived from CD4. CD4 exists in various isoforms. It is understood that any isoform can be selected to achieve a desired function. Exemplary isoforms include isoform 1 (NP_000607.1, GI:10835167), isoform 2 (NP_001181943.1, GI:303522479), isoform 3 (NP_001181944.1, GI:303522485; or NP_001181945.1, GI:303522491; or NP_001181946.1, GI:303522569), and the like. One exemplary isoform sequence, isoform 1, is provided below. In one embodiment, a CAR can have an amino acid sequence comprising the transmembrane domain of CD4 corresponding to amino acids 397 to 418, or fragments thereof. See GenBank NP_000607.1 for reference to domains within CD4, for example, signal peptide, amino acids 1 to 25; extracellular domain, amino acids 26 to 396; transmembrane domain amino acids, 397 to 418; intracellular domain, amino acids 419 to 458. It is understood that additional sequence of CD4 beyond the transmembrane domain of amino acids 397 to 418 can be included in a CAR, if desired. It is further understood that sequences of CD4 that are shorter or longer than a specific dilineated domain can be included in a CAR, if desired. It also is understood that a “CD4 nucleic acid molecule” refers to a polynucleotide encoding a CD4 polypeptide.

| (NP_000607.1; SEQ ID NO: 12) | |
|---|---|
| 1 | MNRGVPFRHL LLVLQLALLP AATQGKKVVL GKKGDTVELT CTASQKKSIQ FHWKNSNQIK |
| 61 | ILGNQGSFLT KGPSKLNDRA DSRRSLWDQG NFPLIIKNLK IEDSDTYICE VEDQKEEVQL |
| 121 | LVFGLTANSD THLLQGQSLT LTLESPPGSS PSVQCRSPRG KNIQGGKTLS VSQLELQDSG |
| 181 | TWTCTVLQNQ KKVEFKIDIV VLAFQKASSI VYKKEGEQVE FSFPLAFTVE KLTGSGELWW |
| 241 | QAERASSSKS WITFDLKNKE VSVKRVTQDP KLQMGKKLPL HLTLPQALPQ YAGSGNLTLA |
| 301 | LEAKTGKLHQ EVNLVVMRAT QLQKNLTCEV WGPTSPKLML SLKLENKEAK VSKREKAVWV |
| 361 | LNPEAGMWQC LLSDSGQVLL ESNIKVLPTW STPVQPMALI VLGGVAGLLL FIGLGIFFCV |
| 421 | RCRHRRRQAE RMSQIKRLLS EKKTCQCPHR FQKTCSPI |

As disclosed herein, mesothelin CARs exemplify CARs that can target a cancer antigen, and CARs directed to other cancer antigens or antigens of pathogens can be generated using similar methods and others well known in the art, as described above. It is understood that domains of the polypeptides described herein can be used in a cancer antigen or pathogen antigen-specific CAR, as useful to provide a desired function such as a signal peptide, antigen binding domain, transmembrane domain, intracellular signaling domain and/or co-stimulatory domain. For example, a domain can be selected such as a signal peptide, a transmembrane domain, an intracellular signaling domain, or other domain, as desired, to provide a particular function to a CAR of the invention. Possible desirable functions can include, but are not limited to, providing a signal peptide and/or transmembrane domain.

In one embodiment, the invention provides CARs directed to mesothelin. In certain non-limiting embodiments, MSLN is human mesothelin having the sequence with an NCBI Reference No: AAV87530.1 (GI:56406362), or fragments thereof, as provided below:

(GenBank AAV87530.1; SEQ ID NO: 13)
MALPTARPLL GSCGTPALGS LLFLLFSLGW VQPSRTLAGE TGQEAAPLDG VLANPPNISS
LSPRQLLGFP CAEVSGLSTE RVRELAVALA QKNVKLSTEQ LRCLAHRLSE PPEDLDALPL
DLLLFLNPDA FSGPQACTHF FSRITKANVD LLPRGAPERQ RLLPAALACW GVRGSLLSEA
DVRALGGLAC DLPGRFVAES AEVLLPRLVS CPGPLDQDQQ EAARAALQGG GPPYGPPSTW
SVSTMDALRG LLPVLGQPII RSIPQGIVAA WRQRSSRDPS WRQPERTILR PRFRREVEKT
ACPSGKKARE IDESLIFYKK WELEACVDAA LLATQMDRVN AIPFTYEQLD VLKHKLDELY
PQGYPESVIQ HLGYLFLKMS PEDIRKWNVT SLETLKALLE VNKGHEMSPQ VATLIDRFVK
GRGQLDKDTL DTLTAFYPGY LCSLSPEELS SVPPSSIWAV RPQDLDTCDP RQLDVLYPKA
RLAFQNMNGS EYFVKIQSFL GGAPTEDLKA LSQQNVSMDL ATFMKLRTDA VLPLTVAEVQ
KLLGPHVEGL KAEERHRPVR DWILRQRQDD LDTLGLGLQG GIPNGYLVLD LSVQEALSGT
PCLLGPGPVL TVLALLLAST LA

In certain embodiments, the extracellular antigen-binding domain of the anti-mesothelin CAR comprises a human anti-mesothelin antibody or an antigen-binding portion thereof described in U.S. Pat. No. 8,357,783, which is herein incorporated by reference in its entirety. In some embodiments, the extracellular antigen-binding domain is derived from a heavy chain variable region and a light chain variable region of an antibody that binds to human mesothelin, for example, antibody m912 as disclosed in Feng et al., Mol. Cancer Therapy 8(5):1113-1118 (2009), which is herein incorporated by reference in its entirety. Antibody m912 was isolated from a human Fab library by panning against recombinant mesothelin. In other embodiments, the extracellular antigen-binding domain is derived from an Fab, for example, from human or mouse Fab libraries.

In certain embodiments, the extracellular antigen-binding domain or an MSLN CAR comprises a heavy chain variable region comprising amino acids having the sequence set forth below.

(SEQ ID NO: 14)

QVQLQESGPGLVKPSETLSLTCTVSGGSVSSGSYYWSWIRQPPGKGLE
WIGYIYYSGSTNYNPSLKSRVTISVDTSKNQFSLKLSSVTAADTAVYY
CAREGKNGAFDIWGQGTMVTVSSASTKGPSVFPLAPSSKSTSGGTAAL
GCLVKDYFPEPVTVSWNSGALTSGVHTFPAVLQSSGLYSLSSVVTVPS
SSLGTQTYICNVNHKPSNTKVDKKVEPKSCDKTSGQAG

The nucleic acid sequence encoding the amino acid sequence above is set forth below.

| (SEQ ID NO: 15) | |
|---|---|
| caggtgcagctgcaggagtccggcccaggactggtgaagccttcggagaccctgtccctc | 60 |
| acctgcactgtctctggtggctccgtcagcagtggtagttactactggagctggatccgg | 120 |
| cagcccccagggaagggactggagtggattgggtatatctattacagtgggagcaccaac | 180 |
| tacaacccctccctcaagagtcgagtcaccatatcagtagacacgtccaagaaccagttc | 240 |
| tccctgaagctgagctctgtgaccgctgcggacacggccgtgtattactgtgcgagagag | 300 |
| gggaagaatggggcttttgatatctggggccaagggacaatggtcaccgtctcttcagcc | 360 |
| tccaccaagggcccatcggtcttccccctggcaccctcctccaagagcacctctgggggc | 420 |
| acagcggccctgggctgcctggtcaaggactacttccccgaaccggtgacggtgtcgtgg | 480 |
| aactcaggcgccctgaccagcggcgtgcacaccttcccggctgtcctacagtcctcagga | 540 |
| ctctactccctcagcagcgtggtgaccgtgccctccagcagcttgggcacccagacctac | 600 |
| atctgcaacgtgaatcacaagcccagcaacaccaaggtggacaagaaagttgagcccaaa | 660 |
| tcttgtgacaaaactagtggccaggccggccac | 693 |

In some embodiments, the extracellular antigen-binding domain comprises a light chain variable region comprising amino acids having the sequence set forth below.

(SEQ ID NO: 16)

DIQMTQSPSSLSASVGDRVTITCRASQSISSYLNWYQQKPGKAPKLLI
YAASSLQSGVPSGFSGSGSGTDFTLTISSLQPEDFATYYCQQSYSTPL
TFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC

The nucleic acid sequence encoding the amino acid sequence above is set forth below.

| (SEQ ID NO: 17) | |
|---|---|
| gacatccagatgacccagtctccatcctccctgtctgcatctgtaggagacagagtcacc | 60 |
| atcacttgccgggcaagtcagagcattagcagctatttaaattggtatcagcagaaacca | 120 |
| gggaaagcccctaagctcctgatctatgctgcatccagtttgcaaagtggggtcccatca | 180 |
| gggttcagtggcagtggatctgggacagatttcactctcaccatcagcagtctgcaacct | 240 |
| gaagattttgcaacttactactgtcaacagagttacagtaccccgctcactttcggcgga | 300 |
| gggaccaaggtggagatcaaacgaactgtggctgcaccatctgtcttcatcttcccgcca | 360 |
| tctgatgagcagttgaaatctggaactgcctctgttgtgtgcctgctgaataacttctat | 420 |
| cccagagaggccaaagtacagtggaaggtggataacgccctccaatcgggtaactcccag | 480 |
| gagagtgtcacagagcaggacagcaaggacagcacctactgcctcagcagcaccctgacg | 540 |
| ctgagcaaagcagactacgagaaacacaaactctacgcctgcgaagtcacccatcagggc | 600 |
| ctgagctcgcccgtcacaaagagcttcaacaggggagagt | |

In some embodiments, the extracellular antigen-binding domain comprises a light chain variable region comprising amino acids having the sequence set forth below.

(SEQ ID NO: 18)

RHQMTQSPSSLSASVGDRVTITCRASQSISSYLNWYQQKPGKAPKLLIY
AASSLQSGVPSGFSGSGSGTDFTLTISSLQPEDFATYYCQQSYSTPLT
FGGGTKVEIKRTVAAPSVFIFPPSDEQLKSGTASVVCLLNNFYPREAKV
QWKVDNALQSGNSQESVTEQDSKDSTYSLSSTLTLSKADYEKHKVY
ACEVTHQGLSSPVTKSFNRGEC

In certain embodiments, the extracellular antigen-binding domain of an MSLN CAR comprises a single-chain variable fragment (scFv). In one specific embodiment, the extracellular antigen-binding domain of a CAR comprises a human scFv. In one embodiment, the human scFv comprises a heavy chain variable region comprising amino acids 1-119 of the MSLN CAR described above (SEQ ID NO:14). In another embodiment, the human scFv of an MSLN CAR comprises a heavy chain variable region comprising amino acids having the sequence set forth below.

(SEQ ID NO: 19)

QVQLQESGPGLVKPSETLSLTCTVSGGSVSSGSYYWSWIRQPPGKGLE
WIGYIYYSGSTNYNPSLKSRVTISVDTSKNQFSLKLSSVTAADTAVYYC
AREGKNGAFDIWGQGTMVTVSSS

In one embodiment, the human scFv comprises a light chain variable region comprising amino acids 1-107 of SEQ ID NO:16. In one embodiment, the human scFv comprises a light chain variable region comprising amino acids 1-107 of SEQ ID NO:18.

In certain embodiments, the human scFv comprises amino acids having the sequence set forth below.

(SEQ ID NO: 20)
Q V Q L Q E S G P G L V K P S E T L S L T C T V S G G S V S S G S Y Y
W S W I R Q P P G K G L E W I G Y I Y Y S G S T N Y N P S L K S R V T
I S V D T S K N Q F S L K L S S V T A A D T A V Y Y C A R E G K N G A
F D I W G Q G T M V T V S S S G G G G S G G G G S G G G G S R H Q M T
Q S P S S L S A S V G D R V T I T C R A S Q S I S S Y L N W Y Q Q K P
G K A P K L L I Y A A S S L Q S G V P S R F S G S G S G T D F T L T I
S S L Q P E D F A T Y Y C Q Q S Y S T P L T F G G G T K V E I K G Q A
G H H H H H H G D Y K D D D D K G

In one embodiment, the nucleic acid sequence encoding the amino acid sequence above is set forth below.

(SEQ ID NO: 21)

atggccttaccagtgaccgccttgctcctgccgctggccttgctgctcca
cgccgccaggccgcaggtgcagctgcaggagtccggcccaggactggtga
agccttcggagaccctgtccctcacctgcactgtctctggtggctccgtc
agcagtggtagttactactggagctggatccggcagcccccagggaaggg
actggagtggattgggtatatctattacagtgggagcaccaactacaacc
cctccctcaagagtcgagtcaccatatcagtagacacgtccaagaaccag
ttctccctgaagctgagctctgtgaccgctgcggacacggccgtgtatta
ctgtgcgagagaggggaagaatggggcttttgatatctggggccaaggga
caatggtcaccgtctcttcaggtggaggcggttcaggcggaggtggctct
ggcggtggcggatcacgacatcagatgacccagtctccatcctccctgtc
tgcatctgtaggagacagagtcaccatcacttgccgggcaagtcagagca
ttagcagctatttaaattggtatcagcagaaaccagggaaagcccctaag
ctcctgatctatgctgcatccagtttgcaaagtggggtcccatcaaggtt
cagtggcagtggatctgggacagatttcactctcaccatcagcagtctgc
aacctgaagattttgcaacttactactgtcaacagagttacagtaccccg
ctcactttcggcggagggaccaaggtggagatcaaacggactgcggc
cgca

In another embodiment, a nucleic acid sequence encoding the amino acid sequence of SEQ ID NO:20 is as provided below. The nucleic acid sequence set forth below is synthetically optimized for codon usage, which can increase the expression of the CAR, as disclosed herein.

(SEQ ID NO: 22)

ATGGCGCTGCCGGTGACCGCGCTGCTGCTGCCGCTGGCGCTGCTGCTGC
ATGCGGCGCGCCCGCAGGTGCAGCTGCAGGAAAGCGGCCCGGGCCTGGT
GAAACCGAGCGAAACCCTGAGCCTGACCTGCACCGTGAGCGGCGGCAGC
GTGAGCAGCGGCAGCTATTATTGGAGCTGGATTCGCCAGCCGCCGGGCA
AAGGCCTGGAATGGATTGGCTATATTTATTATAGCGGCAGCACCAACTA
TAACCCGAGCCTGAAAAGCCGCGTGACCATTAGCGTGGATACCAGCAAA
AACCAGTTTAGCCTGAAACTGAGCAGCGTGACCGCGGCGGATACCGCGG
TGTATTATTGCGCGCGCGAAGGCAAAAACGGCGCGTTTGATATTTGGGG
CCAGGGCACCATGGTGACCGTGAGCAGCGGCGGCGGCGGCAGCGGCGGC
GGCGGCAGCGGCGGCGGCGGCAGCCGCCATCAGATGACCCAGAGCCCGA
GCAGCCTGAGCGCGAGCGTGGGCGATCGCGTGACCATTACCTGCCGCGC
GAGCCAGAGCATTAGCAGCTATCTGAACTGGTATCAGCAGAAACCGGGC
AAAGCGCCGAAACTGCTGATTTATGCGGCGAGCAGCCTGCAGAGCGGCG
TGCCGAGCCGCTTTAGCGGCAGCGGCAGCGGCACCGATTTTACCCTGAC
CATTAGCAGCCTGCAGCCGGAAGATTTTGCGACCTATTATTGCCAGCAG
AGCTATAGCACCCCGCTGACCTTTGGCGGCGGCACCAAAGTGGAAATTA
AACGCACCGCGGCGGCG

In yet another embodiment, a nucleic acid sequence encoding the amino acid sequence of SEQ ID NO:20 is as provided below. The nucleic acid sequence as set forth below is synthetically optimized for codon usage, which can increase the expression of the CAR.

(SEQ ID NO: 23)

atggccCTCCCGGTAACGGCTCTGCTGCTTCCACTCGCACTGCTCTTGC
ATGCTGCCAGACCACAGGTCCAGCTGCAGGAGAGTGGGCCTGGACTGGT
TAAGCCGAGTGAGACACTTTCCTTGACGTGCACTGTGAGCGGGGGAAGT
GTGTCCTCAGGTAGTTATTACTGGTCCTGGATTCGCCAGCCACCAGGAA
AGGGACTGGAGTGGATAGGTTATATCTATTATTCTGGCAGCACTAATTA
CAATCCTTCTCTCAAAAGTAGGGTGACAATTTCAGTGGATACTTCCAAA
AATCAGTTTAGTCTGAAGCTCAGCTCTGTGACAGCTGCTGATACTGCAG
TTTACTACTGCGCCAGGGAGGGGAAGAATGGCGCCTTCGATATTTGGGG
ACAGGGCACTATGGTGACTGTATCAAGCGGAGGCGGTGGCAGCGGCGGG
GGAGGGAGTGGAGGCGGCGGGTCTCGACATCAGATGACACAGAGCCCAT
CATCACTTAGCGCCAGCGTTGGCGACCGGGTTACGATAACATGCAGGGC
TTCCCAATCTATCAGTTCTTATCTGAACTGGTATCAGCAGAAACCAGGT
AAGGCCCCCAAGCTGCTCATCTACGCAGCCTCATCCCTGCAGAGCGGCG
TCCCTAGTCGATTTTCCGGTAGTGGGTCAGGGACAGATTTTACCCTGAC
TATCAGTTCACTGCAGCCCGAGGACTTCGCGACATACTATTGCCAACAG
TCCTATAGTACACCCTTGACATTTGGCGGCGGGACTAAAGTAGAAATTA
AACGCACCgcggccgca

In certain embodiments, the extracellular antigen-binding domain of a CAR comprises a heavy chain variable region CDR1 comprising the amino acids GGSVSSGSYY (SEQ ID NO:24), a heavy chain variable region CDR2 comprising the amino acids IYYSGST (SEQ ID NO:25), and a heavy chain variable region CDR3 comprising the amino acids AREGKNGAFDIW (SEQ ID NO:26). In some embodiments, the extracellular antigen-binding domain comprises a light chain variable region CDR1 comprising the amino acids QSISSY (SEQ ID NO:27), a light chain variable region CDR2 comprising the amino acids AASS (SEQ ID NO:28), and a light chain variable region CDR3 comprising the amino acids QQSYSTPLTF (SEQ ID NO:29). In one non-limiting, exemplary embodiment, the extracellular antigen-binding domain is a human scFv derived from a fully human anti-MSLN antibody m912 as disclosed in Feng et al., Mol. Cancer Therapy 8(5):1113-1118 (2009), which is incorporated herein by reference.

In one embodiment, an exemplary CAR is Mz, which comprises an extracellular antigen binding domain that specifically binds to human mesothelin, a transmembrane domain comprising a CD8 polypeptide, and an intracellular domain comprising a CD3ζ polypeptide. Mz also comprises a signal peptide covalently joined to the N-terminus of the extracellular antigen-binding domain. The signal peptide comprises a CD8 polypeptide comprising amino acids having the sequence MALPVTALLLPLALLLHAARP (SEQ ID NO:30).

In one embodiment, an exemplary CAR is M28z, which comprises an extracellular antigen binding domain that specifically binds to human mesothelin, a transmembrane domain comprising a CD28 polypeptide, and an intracellular domain comprising a CD3ζ polypeptide and a co-stimulatory signaling region comprising a CD28 polypeptide. M28z also comprises a signal peptide covalently joined to the N-terminus of the extracellular antigen-binding domain. The signal peptide comprises a CD8 polypeptide comprising amino acids having the sequence MALPVTALLLPLALLLHAARP (SEQ ID NO:31).

In one embodiment, an exemplary CAR is MBBz, which comprises an extracellular antigen binding domain that specifically binds to human mesothelin, a transmembrane domain comprising a CD8 polypeptide, and an intracellular domain comprising a CD3ζ polypeptide and a co-stimulatory signaling region comprising a 4-1BB polypeptide. MBBz also comprises a signal peptide covalently joined to the N-terminus of the extracellular antigen-binding domain. The signal peptide comprises a CD8 polypeptide comprising amino acids having the sequence MALPVTALLLPLALLLHAARP (SEQ ID NO:32).

### 7.3 Target Antigens

The target antigen is associated with a mammalian disease or disorder. The mammalian disease or disorder as described herein is a cancer or an infection with a pathogen (i.e., pathogen infection). Thus, the target antigen as described herein is a cancer antigen or an antigen of a pathogen (i.e., pathogen antigen).

In certain embodiments of the invention, the mammalian disease or disorder is a cancer and the target antigen is a cancer antigen. A cancer antigen can be uniquely expressed on a cancer cell, or the cancer antigen can be overexpressed in a cancer cell relative to noncancerous cells or tissues. In specific embodiments, the cancer antigen to be bound by a CAR is chosen to provide targeting of the cell expressing the CAR over noncancerous cells or tissues. In one embodiment of the methods of the invention for treating a cancer, an immunostimulatory cell is designed to treat a cancer patient by expressing in the cell a CAR that binds to a suitable cancer antigen of the patient's cancer, as described herein.

The cancer antigen can be a tumor antigen. Any suitable cancer antigen can be chosen based on the type of cancer exhibited by a subject (cancer patient) to be treated. In specific embodiments, the selected cancer antigen is expressed in a manner such that the cancer antigen is accessible for binding by a CAR. Generally, the cancer antigen to be targeted by a cell expressing a CAR is expressed on the cell surface of a cancer cell. However, it is understood that any cancer antigen that is accessible for binding to a CAR is suitable for targeting the CAR expressing cell to the cancer cell. Exemplary cancer antigens and exemplary cancers are provided below in Table 1.

TABLE 1
Cancer Antigens and Corresponding Cancer Targets.

| Antigen targeted | Tumors investigated | References¹ |
|---|---|---|
| B7-H3 | Sarcoma and Neuroblastoma | (1) |
| CD276 | | |
| B7-H6 | Ovarian and several solid cancers | (2-4) |
| Nkp30 | | |
| CAIX | Renal cell carcinoma | (5) |
| Carbonic Anhydrase IX | | |
| CEA | Liver metastasis from Colon cancer, Colon, Pancreas, | (6-20) |
| Carcinoembryonic Antigen | Gastric and Lung cancers | |
| CSPG4 | Melanoma, Mesothelioma, Glioblastoma, | (21-24) |
| Chondroitin sulfate proteoglycan-4 | Osteosarcoma, Breast, Head and Neck cancers | |
| DNAM-1 | Melanoma | (25) |
| DNAX Accessory Molecule | | |
| EpHA2 | Glioblastoma and Lung cancer | (26, 27) |
| Ephrin type A Receptor 2 | | |
| EpCAM | Prostate cancer | (28, 29) |
| Epithelial Cell Adhesion Molecule | | |
| ERBB family | Head and Neck and Breast cancers | (30, 31) |
| ERBB2 | Prostate, Breast, Ovarian and Pancreatic cancers, | (32-48) |
| | Glioblastoma, Meduloblastoma, Osteosarcoma, | |
| | Ewing sarcoma, Neuroectodermal tumor, | |
| | Desmoplastic small round cell tumor and | |
| | Fibrosarcoma | |
| EGFRvIII | Glioma/Glioblastoma | (49-56) |
| Epidermal Growth Factor Receptor | | |
| vIII | | |
| FAP | Tumor associated fibroblast in Lung cancer, | (27, 57-59) |
| Fibroblast Associated Protein | Mesothelioma, Breast and Pancreatic cancers | |
| FRα and β | Ovarian cancer | (60-64) |
| Folate Receptor | | |
| GD2 | Neuroblastoma, Edwing sarcoma, Melanoma | (65-71) |
| Disialoganglioside | | |
| GD3 | Melanoma and other Neuroectodermal tumors | (72, 73) |
| Gp100/HLA-A2 | Melanoma | (74, 75) |
| GPC3 | Hepatocellular carcinoma | (76) |
| Glypican 3 | | |
| HERK-V | Melanoma | (77) |
| MAGE-1/HLA-A1 | Melanoma | (78, 79) |
| Melanoma Antigen E | | |
| IL-11Rα | Osteosarcoma | (80) |
| IL-13Rα2 | Glioma/Glioblastoma | (81-87) |
| | Medullobastoma | |
| Lewis-Y | Ovarian | (88) (89, 90) |
| LMP1 | Nasopharyngeal cancer | (91) |
| Latent Membrane Protein 1 | | |
| L1-CAM | Glioblastoma, Neuroblastoma, Ovarian, Lung and | (92, 93) |
| CD271 L1-Cellular Adhesion | Renal carcinoma | |
| Molecule | | |
| Muc-1 | Prostate and Breast cancers | (43, 94-96) |
| Mucin-1 | | |
| Muc-16 | Ovarian cancer | (97, 98) |
| Mucin-16 | | |
| MSLN | Ovarian, Mesothelioma, Lung cancers | (99-107) |
| Mesothelin | | |
| N-cam | Neuroblastoma | (108) |
| CD56 Neural cell-adhesion | | |
| molecule1 | | |
| NKG2DL | Ovarian | (109, 110) |
| NKG2D Ligands | | |
| PSCA | Prostate cancer | (111-113) |
| Prostate Stem cell Antigen | | |
| PSMA | Prostate | (114-117) |
| Prostate Specific Membrane Antigen | | |
| ROR1 | Epithelial solid tumors | (117, 118) |
| Receptor tyrosine kinase-like Orphan | | |
| Receptor | | |
| TAG72 | Gastrointestinal, Colon and Breast cancers | (119-122) |
| Tumor Associated Glycoprotein 72 | | |
| TRAIL R | Various type of cancer | (123) |
| Trail Receptor | | |
| VEGFR2 | Tumor associated vasculature | (124-127) |
| Vascular Endothelial Growth Factor | | |
| Receptor-2 | | |

¹1. Cheung et al., Hybrid Hybridomics, 22: 209-18 (2003); 2. Zhang et al., J Immunol., 189: 2290-9 (2012); 3. Wu et al., Gene Ther., 22: 675-684 (2015); 4. Wu et al., J Immunol., 194: 5305-11 (2015); 5. Lamers et al., Mol Ther., 21: 904-12 (2013); 6. Darcy et al., Eur J Immunol., 28: 1663-72 (1998); 7. Nolan et al., Clin Cancer Res., 5: 3928-41 (1999); 8. Darcy et al., J Immunol., 164: 3705-12 (2000); 9. Hombach et al., Gene Ther., 6: 300-4 (1999); 10. Haynes et al., J Immunol., 166: 182-7 (2001); 11. Haynes et al., J Immunol., 169: 5780-6 (2002); 12. Schirrmann et al., Cancer Gene Ther., 9: 390-8 (2002); 13. Arakawa et al., Anticancer Res. 2002; 22: 4285-9. 14. Gyobu et al., Cancer Res., 64: 1490-5 (2004); 15. Shibaguchi et al., Anticancer Res., 26: 4067-72 (2006); 16. Emtage et al., Clin Cancer Res. 14: 8112-22 (2008); 17. Chmielewski et al., Gastroenterology, 143: 1095-107 e2 (2012); 18. Chmielewski et al., Gene Ther., 20: 177-86 (2013); 19. Burga et al., Cancer Immunol Immunother., 64: 817-29 (2015); 20. Katz et al. Clin Cancer Res., 21: 3149-59 (2015); 21. Beard et al., J Immunother Cancer, 2: 25 (2014); 22. Burns et al., Cancer Res., 70: 3027-33 (2010); 23. Geldres et al., Clin Cancer Res., 20: 962-71 (2014); 24. Schmidt et al., Proc Natl Acad Sci USA, 108: 2474-9(2011); 25. Wu et al., Cancer Immunol Immunother., 64: 409-18 (2015); 26. Chow et al., Mol Ther., 21: 629-37 (2013); 27. Kakarla et al., Mol Ther., 21: 1611-20 (2013); 28. Shirasu et al., J Biomed Biotechnol., 2012: 853879 (2012); 29. Deng et al., BMC Immunol., 16: 1 (2015); 30. Davies et al., Mol Med., 18: 565-76 (2012); 31. Papa et al., Methods Mol Biol., 1317: 365-82 (2015); 32. Stancovski et al., J Immunol., 151: 6577-82 (1993); 33. Moritz et al., Proc Natl Acad Sci USA, 91: 4318-22 (1994); 34. Haynes et al., Cancer Immunol Immunother., 47: 278-86 (1999); 35. Pinthus et al., Cancer Res., 63: 2470-6 (2003); 36. Ahmed et al., Cancer Res., 67: 5957-64 (2007); 37. Li et al., Cancer Gene Ther. 15: 382-92 (2008); 38. Wang et al., Clin Cancer Res., 15: 943-50 (2009); 39. Ahmed et al., Mol Ther., 17: 1779-87 (2009); 40. Zhao et al., J Immunol., 183: 5563-74 (2009); 41. Ahmed et al., Clin Cancer Res., 16: 474-85 (2010); 42. Duong et al., Immunotherapy, 3: 33-48 (2011); 43. Wilkie et al., J Clin Immunol., 32: 1059-70 (2012); 44. Lanitis et al., PLoS One, 7: e49829 (2012); 45. Maliar et al., Gastroenterology, 143: 1375-84 e1-5 (2012); 46. Rainusso et al., Cancer Gene Ther., 19: 212-7 (2012); 47. Sun et al., Breast Cancer Res., 16: R61 (2014); 48. Ahmed et al., J Clin Oncol., 33: 1688-96 (2015); 49. Ohno et al., Cancer Sci., 101: 2518-24 (2010); 50. Morgan et al., Hum Gene Ther., 23: 1043-53 (2012); 51. Choi et al., J Clin Neurosci., 21: 189-90 (2014); 52. Ohno et al., J Immunother Cancer, 1: 21 (2013); 53. Shen et al., J Hematol Oncol., 6: 33 (2013); 54. Sampson et al., Clin Cancer Res., 20: 972-84 (2014); 55. Miao et al., PLoS One, 9: e94281 (2014); 56. Johnson et al., Sci Transl Med., 7: 275ra22 (2015); 57. Petrausch et al., BMC Cancer, 12: 615 (2012); 58. Schuberth et al., J Transl Med., 11: 187 (2013); 59. Wang et al., Cancer Immunol Res., 2: 154-66 (2014); 60. Parker et al., Hum Gene Ther., 11: 2377-87 (2000); 61. Kershaw et al., Clin Cancer Res., 12: 6106-15 (2006); 62. Song et al., Cancer Res., 71: 4617-27 (2011); 63. Kandalaft et al., J Transl Med., 10: 157 (2012); 64. Song et al., Oncotarget, (2015); 65. Krause et al., J Exp Med., 188: 619-26 (1998); 66. Rossig et al., Int J Cancer, 94: 228-36 (2001); 67. Pule et al., Nat Med., 14: 1264-70 (2008); 68. Yvon et al., Clin Cancer Res., 15: 5852-60 (2009); 69. Louis et al., Blood, 118: 6050-6 (2011); 70. Kailayangiri et al., Br J Cancer, 106: 1123-33 (2012); 71. Singh et al., Cancer Immunol Res., 2: 1059-70 (2014); 72. Yun et al., Neoplasia, 2: 449-59 (2000); 73. Lo et al., Clin Cancer Res., 16: 2769-80 (2010); 74. Zhang et al., Immunol Cell Biol., 91: 615-24 (2013); 75. Zhang et al., Sci Rep., 4: 3571 (2014); 76. Gao et al., Clin Cancer Res., 20: 6418-28 (2014); 77. Krishnamurthy et al., Clin Cancer Res., 21: 3241-51 (2015); 78. Willemsen et al., Gene Ther., 8: 1601-8 (2001); 79. Willemsen et al., J Immunol., 174: 7853-8 (2005); 80. Huang et al., Cancer Res., 72: 271-81 (2012); 81. Stastny et al., J Pediatr Hematol Oncol., 29: 669-77 (2007); 82. Chang et al., Cytotherapy, 9: 771-84 (2007); 83. Lazovic et al., Clin Cancer Res., 14: 3832-9 (2008); 84. Kong et al., Clin Cancer Res., 18: 5949-60 (2012); 85. Hegde et al., Mol Ther., 21: 2087-101 (2013); 86. Krebs et al., Cytotherapy, 16: 1121-31 (2014); 87. Brown et al., Clin Cancer Res., (2015); 88. Westwood et al., Proc Natl Acad Sci USA, 102: 19051-6 (2005); 89. Westwood et al., J Immunother., 32: 292-301 (2009); 90. Neeson et al., Gene Ther., 17: 1105-16 (2010); 91. Tang et al., J Biomed Res., 28: 468-75 (2014); 92. Park et al., Mol Ther., 15: 825-33 (2007); 93. Hong et al., J Immunother., 37: 93-104 (2014); 94. Wilkie et al., J Immunol., 180: 4901-9 (2008); 95. Bakhtiari et al., Hybridoma (Larchmt)., 28: 85-92 (2009); 96. Sanchez et al., Prostate Cancer Prostatic Dis., 16: 123-31, S1 (2013); 97. Chekmasova et al., Discov Med., 9: 62-70 (2010); 98. Koneru et al., Oncoimmunology, 4: e994446 (2015); 99. Carpenito et al., Proc Natl Acad Sci USA, 106: 3360-5 (2009); 100. Zhao et al., Cancer Res., 70: 9053-61 (2010); 101. Lanitis et al., Mol Ther., 20: 633-43 (2012); 102. Riese et al., Cancer Res., 73: 3566-77 (2013); 103. Moon et al., Clin Cancer Res., 20: 4262-73 (2014); 104. Guedan et al., Blood, 124: 1070-80 (2014); 105. Beatty, Oncoimmunology, 3: e28327 (2014); 106. Adusumilli et al., Sci Transl Med., 6: 261ra151 (2014); 107. Wang et al., Cancer Immunol Res., 3: 815-26 (2015); 108. Gilham et al., J Immunother., 25: 139-51 (2002); 109. Barber et al., J Immunol., 183: 6939-47 (2009); 110. Song et al., Hum Gene Ther., 24: 295-305 (2013); 111. Morgenroth et al., Prostate, 67: 1121-31 (2007); 112. Hillerdal et al., BMC Cancer, 14: 30 (2014); 113. Abate-Daga et al., Hum Gene Ther., 25: 1003-12 (2014); 114. Maher et al., Nat Biotechnol., 20: 70-5 (2002); 115. Ma et al., Prostate, 61: 12-25 (2004); 116. Gade et al., Cancer Res., 65: 9080-8 (2005); 117. Hudecek et al., Clin Cancer Res., 19: 3153-64 (2013); 118. Deniger et al., PLoS One, 10: e0128151 (2015); 119. Hombach et al., Gastroenterology, 113: 1163-70 (1997); 120. McGuinness et al., Hum Gene Ther., 10: 165-73 (1999); 121. Patel et al., Cancer Gene Ther. 7: 1127-34 (2000); 122. Sharifzadeh et al., Cancer Lett., 334: 237-44 (2013); 123. Kobayashi et al., Biochem Biophys Res Commun. 453: 798-803 (2014); 124. Chinnasamy et al., Clin Cancer Res., 18: 1672-83 (2012); 125. Kanagawa et al., Cancer Gene Ther., 20: 57-64 (2013); 126. Chinnasamy et al., Cancer Res., 73: 3371-80 (2013); 127. Wang et al., Gene Ther., 20: 970-8 (2013); references 128-175, additional references of cancer antigens and corresponding cancer antigen targets; 128. Ordonez, Am J Surg Pathol., 27: 1418-28 (2003); 129. Dennis et al., Clin Cancer Res., 11: 3766-72 (2005); 130. Alvarez et al., Nanomedicine, 4: 295-301 (2008); 131. Rizk et al., Cancer Epidemiol Biomarkers Prev., 21: 482-6 (2012); 132. Ordonez, Mod Pathol., 16: 192-7 (2003); 133. Frierson et al., Hum Pathol., 34: 605-9 (2003); 134. Tchou et al., Breast Cancer Res Treat., 33: 799-804 (2012); 135. Parinyanitikul et al., Clin Breast Cancer, 13: 378-84 (2013); 136. Wang et al., J Int Med Res., 40: 909-16 (2012); 137. Li et al., Breast Cancer Res Treat., 147: 675-84 (2014); 138. Ordonez et al., Hum Pathol., 45: 1529-40 (2014); 139. Tozbikian et al., PLoS One, 9: e114900 (2014); 140. Bayoglu et al., Biomed Pharmacother., 70: 190-5 (2015); 141. Einama et al., Br J Cancer, 107: 137-42 (2012); 142. Baba et al., J Surg Oncol., 105: 195-9 (2012); 143. Ito et al., Oncol Rep., 31: 27-33 (2014); 144. Hassan et al., Am J Clin Pathol., 124: 838-45 (2005); 145. Yu et al., J Cancer, 1: 141-9 (2010); 146. Kawamata et al., Int J Oncol., 41: 2109-18 (2012); 147. Nomura et al., Int Surg., 98: 164-9 (2013); 148. Argani Pedram et al., Clin Cancer Res., 7: 3862-8 (2001); 149. Swierczynski et al., Hum Pathol. 35: 357-66 (2004); 150. Inami et al., Oncol Rep., 20: 1375-80 (2008); 151. Frank et al., Am J Clin Pathol., 142: 313-9 (2014); 152. Scales et al., Mol Cancer Ther., 13: 2630-40 (2014); 153. Liebig et al., Cancer Lett., 223: 159-67 (2005); 154. Kawamata et al., J Gastroenterol., 49: 81-92 (2014); 155. Miettinen et al., Am J Surg Pathol., 27: 150-8 (2003); 156. Ordonez, Am J Surg Pathol., 27: 1031-51 (2003); 157. Ordonez, Mod Pathol. 19: 417-28 (2006); 158. Kushitani et al., Pathol Int., 57: 190-9 (2007); 159. Pu et al., Diagn Cytopathol., 36: 20-5 (2008); 160. Kachala et al., Clin Cancer Res., 20: 1020-8 (2014); 161. Anish et al., Oncotarget, (2015); 162. Pan et al., Hum Pathol., 34: 1155-62 (2003); 163. Yuanbin et al., 2014 ASCO Annual Meeting, (2014); 164. Ordonez, Hum Pathol., 35: 697-710 (2004); 165. Galloway et al., Histopathology, 48: 767-9 (2006); 166. Roe et al., Lung Cancer, 61: 235-43 (2008); 167. Tan et al., Hum Pathol., 41: 1330-8 (2010); 168. Servais et al., Clin Cancer Res., 18: 2478-89 (2012); 169. Drapkin et al., Hum Pathol., 35: 1014-21 (2004); 170. Rosen et al., Gynecol Oncol. 99: 267-77 (2005); 171. Hassan et al., Appl Immunohistochem Mol Morphol. 13: 243-7 (2005); 172. Cao et al., Int J Gynecol Pathol, 24: 67-72 (2005); 173. Yen et al., Clin Cancer Res., 12: 827-31 (2006); 174. Dainty et al., Gynecol Oncol., 105: 563-70 (2007); 175. Obulhasim et al., Eur J Gynaecol Oncol., 31: 63-71 (2010).

Suitable antigens include, but are not limited to, mesothelin (MSLN), prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (LlCAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF- R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Rα, Latent Membrane Protein 1 (LMP1), Neural cell-adhesion molecule (N-CAM/CD56), and Trail Receptor (TRAIL R). It is understood that these or other cancer antigens can be utilized for targeting by a cancer antigen CAR.

In certain embodiments of the invention, the cancer antigen is mesothelin. In some embodiments of the invention, the CAR is designed to bind to and target cancer cells expressing mesothelin. Mesothelin (MSLN) is an immunogenic cell surface antigen (Ho et al., Clin. Cancer Res. 11:3814-3820 (2005); Hassan et al., Eur. J. Cancer 44:46-53 (2008)) that is highly expressed in solid cancers (Hassan et al., R. & Ho, M. Mesothelin targeted cancer immunotherapy. Eur. J. Cancer 44, 46-53 (2008); Zervos et al., Curr. Opin. Pulm. Med. 14:303-309 (2008); Palumbo et al., Curr. Med. Chem. 15:855-867 (2008); Roe et al., Lung Cancer 61:235-243 (2008); Pass et al., Ann. Thorac. Surg. 85:265-272 (2008); Rodriguez Portal et al., Cancer Epidemiol. Biomarkers Prey. 18(2):646-650 (2009)). MSLN is involved in cell proliferation (Bharadwaj et al., Mol. Cancer Res. 6:1755-1765 (2008)), adhesion (Uehara et al., Mol. Cancer Res. 6:186-193 (2008); Kaneko et al., J. Biol. Chem. 284:3739-3749 (2009)), invasion (Servais et al., Clin. Cancer Res. 18:2478-2489 (2012); Wang et al., J. Int. Med. Res. 40:2109-2116 (2012); Wang et al., J. Int. Med. Res. 40:909-916 (2012)), cell signaling (Uehara et al., N., Mol. Cancer Res. 6:186-193 (2008)), and metastasis (Wu et al., Clin. Cancer Res. 14:1938-1946 (2008)). Studies have demonstrated that serum soluble MSLN-related peptide (SMRP) secreted by MSLN-expressing tumors can be measured in both humans (Pass et al., Ann. Thorac. Surg. 85:265-272 (2008); Cancer Epidemiol. Biomarkers Prey. 18(2):646-650 (2009); Robinson et al., Lung Cancer 49 Suppl 1:S109-5111 (2005); Tajima et al., Anticancer Res. 28:3933-3936 (2008); Park et al., Am. J. Respir. Crit. Care Med. 178:832-837 (2008); Segawa et al., Biochem. Biophys. Res. Commun. 369:915-918 (2008); Amati et al., Cancer Epidemiol. Biomarkers Prey. 17:163-170 (2008); van den Heuvel et al., Lung Cancer 59, 350-354 (2008); Rizk et al., Cancer Epidemiol. Biomarkers Prev. 21:482-486 (2012)) and mice, and has been shown to correlate with therapy response and prognosis. In normal tissues, MSLN is expressed only in the pleura, pericardium, and peritoneum, at low levels (Hassan et al., Eur. J. Cancer 44:46-53 (2008); Bera et al., Mol. Cell. Biol. 20:2902-2906 (2000)). The anti-MSLN recombinant immunotoxin SS1P has shown in vivo specificity and significant antitumor activity in patients (Kelly et al., Mol. Cancer Ther. 11:517-525 (2012); Hassan et al., Clin. Cancer Res. 13:5144-5149 (2007)). In a pancreatic cancer vaccine trial, patients with survival advantage had consistent CD8⁺ T cell responses to MSLN associated with vaccine-induced delayed-type hypersensitivity response (Thomas et al., J. Exp. Med. 200:297-306 (2004)). Specific T cell epitopes derived from MSLN were shown to activate human T cells to efficiently lyse human tumors expressing MSLN (Yokokawa et al., Clin. Cancer Res. 11:6342-6351 (2005)).

MSLN-specific CARs have shown efficacy against ovarian cancer, malignant pleural mesothelioma (MPM), and triple-negative breast cancer (TNBC) in both in vitro and in vivo settings (Lanitis et al., Mol. Ther. 20:633-643 (2012); Moon et al., Clin. Cancer Res. 17:4719-4730 (2011); Zhao et al., Cancer Res. 70:9053-9061 (2010); Riese et al., Cancer Res. 73:3566-3577 (2013); Tchou et al., Breast Cancer Res. Treat. 133:799-804 (2012)). Two Phase I clinical trials have used anti-MSLN CAR-transduced T cells. An NCI Phase I clinical trial (ClinicalTrials.gov record NCT01583686) treats metastatic or unresectable cancers that express MSLN with CAR T cells, in combination with myeloablative chemotherapy and/or aldesleukin (an IL-2 analogue) to augment CAR T cell persistence. A University of Pennsylvania Phase I clinical trial (ClinicalTrials.gov record NCT01355965) gives mesothelioma patients 1 to 3 doses of MSLN-targeted CAR T cells. In the latter study, a human anti-mouse antibody (HAMA) response was observed in the third treated patient (Maus et al., Cancer Immunol. Res. 1(1):26-31 (2013)). In one embodiment, a MSLN-targeted CAR is derived from a human Fab (Feng et al., Mol. Cancer Ther. 8:1113-1118 (2009)), and thus, affords a much decreased risk of immunogenicity, compared with CARs derived from murine antibodies (see Maus et al., Cancer Immunol. Res. 1(1):26-31 (2013)).

In certain embodiments of the invention, the mammalian disease or disorder is an infection with a pathogen and the target antigen is an antigen of the pathogen. In specific embodiments, the pathogen is a human pathogen. In specific embodiments, the pathogen is a virus, a bacterium, a fungus, a protozoan, a helminth, or a protist.

Such a pathogen antigen can be uniquely expressed on a pathogen, or the pathogen antigen can be overexpressed on a pathogen or in a pathogen infected tissue or cell relative to cells or tissues that are not infected with the pathogen. Generally, a pathogen antigen is uniquely expressed by the pathogen or a pathogen infected cell or tissue and is not expressed in an uninfected cell or tissue. In specific embodiments, the pathogen antigen to be bound by the CAR is chosen to provide targeting of the cell expressing the CAR over cells or tissues that are not infected with the pathogen. In one embodiment of the methods of the invention for treating a pathogen infection, an immunostimulatory cell is designed to treat a patient with a pathogen infection by expressing in the cell a CAR that binds to an antigen of the pathogen infecting the patient, as described herein.

Any suitable pathogen antigen can be chosen based on the type of pathogen infection exhibited by a subject (patient with a pathogen infection) to be treated. In specific embodiments, the selected pathogen antigen is expressed in a manner such that the pathogen antigen is accessible for binding by the CAR. Generally, the pathogen antigen to be targeted by a cell expressing a CAR is expressed on the surface of the pathogen or the surface of an infected cell or tissue of the subject. However, it is understood that any pathogen antigen that is accessible for binding to a CAR is suitable for targeting the CAR expressing cell to the site of a pathogen infection or infected tissue.

In a specific embodiment, the pathogen is a virus. In a specific embodiment, the target antigen is of a virus that is a human pathogen, and in a particular embodiment, such a viral antigen of a human pathogen is one that can elicit an immune response in a human patient infected with the virus. Exemplary viruses and their viral antigens that can be targeted include, but are not limited to, those provided below in Table 2.

TABLE 2
Viruses and Viral Antigens

| Virus | Viral Antigen | Reference¹ |
|---|---|---|
| human immunodeficiency | group-specific antigen (gag) protein (p55, p24, | Mitsuya, 1990; |
| virus (HIV) | or p18), envelope glycoprotein (env) (gp160, | Fauci, 1998; |
| | gp120 or gp41) or reverse transcriptase (pol) | Fauci, 1988; |
| | (p66 or p31) | Rosenberg, |
| | | 1997 |
| hepatitis B virus (HBV) | HBV envelope protein S, M or L | Krebs, 2013 |
| hepatitis C virus (HCV) | core protein, envelope protein E1 or E2, | Ashfaq (2011); |
| | nostructural protein NS2, NS3, NS4 (NS4A or | Sillanpää |
| | NS4B), NS5 (NS5A or NS5B) | (2009); |
| | | Dawson (2012) |
| herpes simplex virus (HSV) | gE, gI, gB, gD, gH, gL, gC, gG, gK or gM | Polcicova, |
| | | 2005; Bennett, |
| | | 1996 |
| varicella zoster virus or | gE or gI | Polcicova, |
| (VZV) | | 2005 |
| adenovirus | hexon protein or penton protein | Gerdemann, |
| | | 2013 |
| cytomegalovirus (CMV) | pp65, immediate early (IE) antigen or IE1 | Gerdemann, |
| | | 2013; Rooney, |
| | | 2012 |
| Epstein-Barr virus (EBV) | LMP2 (latent membrane protein 2), EBNA1 | Gerdemann, |
| | (Epstein-Barr nuclear antigen 1) or immediate | 2013; Rooney, |
| | early protein BZLF1 (also known as Zta, | 2012 |
| | ZEBRA, EB1) | |

¹Mitsuya et al., Science 249: 1533-1544 (1990); Fauci et al., Harrison's Principles of Internal Medicine, 14th ed., pp. 1814-1816, McGraw-Hill, San Francisco CA (1998); Fauci, Science 239: 617-622 (1988); Rosenberg et al., Science 278: 1447-1450 (1997); Krebs et al., Gastroenterol. 145: 456-465 (2013); Ashfaq et al., Virol. J. 8: 161 (doi: 10.1186/1743-422X-8-161); Sillanpää et al., Virol. J. 6: 84 (doi: 10.1186/1743-422X-6-84); Dawson, Antiviral Therap. 17: 1431-1435 (2012); Polcicova et al., J. Virol. 79: 11990-12001 (2005); Bennett et al., Cecil Textbook of Medicine, 20th ed., p. 1770, W. B. Saunders, Philadelphia PA (1996); Gerdemann et al., Mol. Ther. 21: 2113-2121 (2013); Rooney et al., Mol. Ther. Nucleic Acids 1: e55, doi: 10.1038/mtna.2012.49 (2012)

In a specific embodiment in the case of HBV, the S domain of an S, M or L envelope protein is targeted (see Krebs et al., supra, 2013). In another specific embodiment in the case of HSV, the extracellular domain of gE is targeted (see Polcicova et al., supra, 2005). It is understood that a person skilled in the art can readily determine a viral antigen, or domain of a viral antigen, suitable for targeting by an immunostimulatory cell of the invention.

It is further understood that reference to a virus, such as those listed in Table 2, includes different strains or types of the same virus. For example, HSV exists as herpes simplex virus type 1 (HSV-1) and herpes simplex virus type 2 (HSV-2), which can be distinguished by the respective glycoprotein G (gG) (Bennett et al., Cecil Textbook of Medicine, 20th ed., p. 1770, W. B. Saunders, Philadelphia PA (1996)). In a particular embodiment, the viral antigen can be selected such that the antigen is common to different strains or types of the same virus or is a distinct antigen specific to a particular strain or type of virus, such as for HSV-1 and HSV-2.

In another specific embodiment, the pathogen is a bacterium, such as a mycobacterium or Chlamydia trachomatis.

In another specific embodiment, the pathogen is a fungus, such as Cryptococcus neoformans, Pneumocystis jiroveci, a Candida, or an invasive fungus.

In another specific embodiment, the pathogen is a protozoan, such as Entamoeba histolytica, Plasmodium, Giardia lamblia, or Trypanosoma brucei.

In another specific embodiment, the pathogen is a helminth, such as Ascaris, Trichuris, or hookworm.

In another specific embodiment, the pathogen is a protist, such as Toxoplasma gondii.

### 7.4. Dominant Negative Forms of an Inhibitor of a Cell-Mediated Immune Response

According to some embodiments of the invention, an immunostimulatory cell, such as a T cell, is engineered to express a dominant negative form of an inhibitor of a cell-mediated immune response.

Malignant cells adapt to generate an immunosuppressive microenvironment that protects the cells from immune recognition and elimination (Sharpe et al., Dis. Model Mech. 8:337-350 (2015)). The immunosuppressive microenvironment puts limitations on immunotherapy methods. The present invention addresses this limitation by expressing in an immunostimulatory cell a dominant negative form of an inhibitor of a cell-mediated immune response.

An inhibitor of a cell-mediated immune response of the immunostimulatory cell refers to a molecule that acts to inhibit or suppress the immune response affected by the immunostimulatory cell. In one embodiment, the inhibitor of a cell-mediated immune response is an immune checkpoint inhibitor, also referred to as a checkpoint blockade.

Immune checkpoint pathways are inhibitory pathways that suppress the immune response of an immune cell. The pathways deliver negative signals to the immune cells, such as T cells, and attenuate TCR-mediated signals, leading to decreased cell proliferation, cytokine production and cell cycle progression (see Pardoll, Nat. Rev. 12:252-264 (2012); Wu et al., Int. J. Biol. Sci. 8:1420-1430 (2012)). The immune checkpoint inhibitor pathway generally involves a ligand-receptor pair. Exemplary immune checkpoint inhibitor pathway receptors include, for example, PD-1, CTLA-4, BTLA, TIM-3, LAG-3, CD160, TIGIT, LAIR1, 2B4, and the like (see Chen et al., Nat. Rev. Immunol. 13(4):227-242 (2013)). The corresponding ligands for these receptors include, for example, PD-L1 (for PD-1); PD-L2 (for PD-1); CD80, CD86 (for CTLA-4); HVEM (for BTLA); Galectin-9, HMGB1 (for TIM-3); MHC II (for LAG-3); HVEM (for CD160); CD155, CD112, CD113 (for TIGIT); C1q, collagen (for LAIR1); CD48 (for 2B4), and the like (Chen et al., supra, 2013). Expression of a dominant negative form in the immunostimulatory cell, such as a T cell, provides for inhibition of a checkpoint inhibitor pathway that is intrinsic to the cell.

In one embodiment of the invention, a dominant negative form of an immune checkpoint inhibitor pathway receptor is provided, as disclosed herein.

A dominant negative form of an inhibitor of a cell-mediated immune response that is a cell-surface receptor such as an immune checkpoint inhibitor pathway receptor can be generated by deleting some portion of the receptor to prevent intracellular signaling, thereby suppressing the immune checkpoint pathway and sustaining activation of the immunostimulatory cell, such as a T cell. A dominant negative form of the invention is a polypeptide comprising (a) at least a portion of an extracellular domain of an immune checkpoint inhibitor, where the portion comprises the ligand binding region, and (b) a transmembrane domain, where the polypeptide is a dominant negative form of the immune checkpoint inhibitor. Generally, a dominant negative form of an inhibitor of an immune checkpoint inhibitor pathway receptor retains most or all of an extracellular domain of the receptor such that the extracellular domain retains sufficient protein interaction activity to bind to its respective ligand. Thus, in a specific embodiment, a polypeptide encoding a dominant negative form comprises substantially all of an extracellular domain of an immune checkpoint inhibitor. It is understood that a polypeptide comprising “substantially all” of an extracellular domain includes a polypeptide that comprises the entire extracellular domain or a portion of the extracellular domain in which one to a few amino acids have been deleted from the N-terminus and/or C-terminus of the extracellular domain, for example deletion of 1, 2, 3, 4, or 5 amino acids from the N-terminus and/or C-terminus, so long as the remaining portion of the extracellular domain retains sufficient protein interaction activity to bind to its respective ligand. A dominant negative form of the invention generally also lacks some portion or all of a signaling domain, such as the intracellular/cytoplasmic domain, such that the dominant negative form has reduced activity or is inactive for signaling in the immune checkpoint pathway. Without being bound by a particular mechanism or theory, binding of the ligand to the dominant negative form decreases binding of the ligand to the intact endogenous receptor, and/or the dominant negative form complexes with signaling molecules, including the endogenous receptor, resulting in decreased signaling of an immune checkpoint pathway.

A dominant negative form of the invention generally has certain functional characteristics including, but not limited to, the ability to be expressed at the cell surface of an immunostimulatory cell, such as a T cell, the ability to bind to its respective ligand, and the inability or reduced ability to propagate an intracellular signal of an immune checkpoint pathway. One skilled in the art can readily generate a dominant negative form of an inhibitor of a cell-mediated immune response by engineering the inhibitor to have such functional characteristics. In one embodiment, a dominant negative form is constructed to retain the extracellular domain of inhibitor of a cell-mediated immune response, or at least a sufficient portion of the extracellular domain to retain ligand binding activity. In an exemplary embodiment, a dominant negative form can be constructed using the extracellular domain of an inhibitor of a cell-mediated immune response, including, but not limited to, the extracellular domains of PD-1, CTLA-4, BTLA, TIM-3, LAG-3, CD160, TIGIT, LAIR1, 2B4, as disclosed herein. One skilled in the art will readily understand that it is not required to retain the entire extracellular domain of an inhibitor of a cell-mediated immune response, and that deletions from the N-terminus and/or C-terminus of the extracellular domain can be introduced so long as ligand binding activity is retained. One skilled in the art can readily determine the appropriateness of such N-terminal and/or C-terminal deletions based on the analysis of the receptor sequence to identify protein motifs known to provide ligand binding activity (see, for example, ExPASγ (expasy.org), in particular PROSITE (prosite.expasy.org)). In addition or alternatively, suitable N-terminal and/or C-terminal deletions can be determined empirically by introducing deletions in a polypeptide and measuring binding activity for the respective ligand. Thus, one skilled in the art can readily determine an appropriate sequence of an inhibitor of a cell-mediated immune response to provide ligand binding activity to a dominant negative form of the invention.

It is understood that, whether an entire extracellular domain or a portion of the extracellular domain of a receptor is used in a dominant negative form, additional sequences can optionally be included in the extracellular domain of the dominant negative form. Such additional sequences can be derived from the parent polypeptide of the dominant negative form, or the additional sequences can be derived from a different polypeptide. Such a polypeptide comprising sequences from a parent polypeptide and a different polypeptide is a non-naturally occurring, chimeric polypeptide. For example, a signal peptide or leader peptide is generally included so that the dominant negative form will be expressed at the cell surface of the immunostimulatory cell such as a T cell. It is understood that, once a polypeptide containing a signal peptide is expressed at the cell surface, the signal peptide has generally been proteolytically removed during processing of the polypeptide in the endoplasmic reticulum and translocation to the cell surface. Thus, a polypeptide such as a dominant negative form is generally expressed at the cell surface as a mature protein lacking the signal peptide, whereas the precursor form of the polypeptide includes the signal peptide. The signal peptide can be the naturally occurring signal peptide of the receptor, or alternatively can be derived from a different protein. Exemplary signal peptides are described herein, including those described herein as being suitable for a CAR. To additionally provide expression at the cell surface, the dominant negative form will generally include a transmembrane domain that provides for retention of the dominant negative form at the cell surface. The transmembrane domain can be the naturally occurring transmembrane domain of the receptor, or alternatively can be derived from a different protein. In a particular embodiment, the transmembrane domain derived from another protein is derived from another receptor expressed on the cell surface of the immunostimulatory cell such as a T cell. Exemplary transmembrane domains are described herein, including those described herein as being suitable for a CAR.

In the case of an immune checkpoint pathway receptor, generally the signaling domain resides within the intracellular/cytoplasmic domain. The signaling activity of an immune checkpoint pathway receptor is generally mediated by protein-protein interactions with cell surface receptor(s) and/or intracellular signaling molecules. In one embodiment, a dominant negative form lacks the entire intracellular domain, or a portion thereof, that functions in propagating the signal of an immune checkpoint pathway. It is understood that it is not necessary to delete the entire intracellular domain of the receptor so long as a sufficient portion of the intracellular signaling domain is deleted to inhibit or reduce signaling from the dominant negative form. In addition or alternatively, mutations can be introduced into the intracellular signaling domain to inhibit or reduce signaling from the dominant negative form. In addition or alternatively, a heterologous sequence with no signaling activity can be substituted for the intracellular signaling domain of the receptor to generate a dominant negative form. One skilled in the art will readily understand that these and other well known methods can be utilized to generate a dominant negative form of the invention.

One exemplary embodiment of a dominant negative form of an immune checkpoint inhibitor is a dominant negative form of PD-1. As described in Cherkassky et al., The Journal of Clinical Investigation 126(8):3130-3144 (2016), a dominant negative form of PD-1 was co-expressed in a CAR T cell with a mesothelin CAR and found to increase tumor elimination and prolong mouse survival. A dominant negative form of PD-1 is exemplary of a dominant negative form of an inhibitor of a cell-mediated immune response, including an immune checkpoint inhibitor. The results disclosed therein indicate that co-expressing a dominant negative form of an inhibitor of a cell-mediated immune response can enhance the effectiveness of a CAR T cell, or other immunostimulatory cell, expressing a cancer antigen CAR. It is understood that a PD-1 dominant negative form as disclosed herein is exemplary. Based on the teachings disclosed herein, one skilled in the art can readily prepare a dominant negative form of an inhibitor of a cell-mediated immune response, including an immune checkpoint pathway receptor.

As described herein, a dominant negative form of an inhibitor of a cell-mediated immune response is designed to have reduced or inhibited intracellular signaling. The dominant negative forms of the invention are generally based on inhibiting a receptor of an immune checkpoint pathway, which function to inhibit activation of an immunostimulatory cell, such as T cell, for example, cell proliferation, cytokine production and/or cell cycle progression. The dominant negative forms of the invention are designed to remove the intracellular signaling domain, or a portion thereof, so that the signaling ability of the receptor is reduced or inhibited. The dominant negative form also functions to inhibit signaling of the endogenous receptor. In a particular embodiment, the reduced or inhibited signaling overcomes the checkpoint blockade, resulting in sustained signaling and activation of the immunostimulatory cell, such as a T cell. It is understood that the signaling activity of the dominant negative form can be completely knocked out or partially knocked out, so long as the partial reduction in activity is sufficient for the effect of providing enhanced activation of the immunostimulatory cell, in comparison to the absence of the dominant negative form. Also, the dominant negative form is not required to result in complete inactivation of signaling from the endogenous receptor but can reduce the activation of the endogenous receptor sufficient to overcome the checkpoint blockade and allow activation of the immunostimulatory cell, such as a T cell. One skilled in the art can readily determine the effect of a dominant negative form on the activity of a parent receptor using assay methods well known in the art, including assays using in vivo models, such as animal models, to assess the effect of the dominant negative form on the effectiveness of CAR expressing cells, as disclosed herein.

As with a CAR for use in the invention, optional linker or spacer sequences can be included in a dominant negative form, for example, a linker or spacer between a signal peptide and the extracellular ligand binding domain, particularly when heterologous sequences are fused. A linker or spacer can also optionally be included between the extracellular ligand binding domain and the transmembrane domain. Similarly, a linker or spacer can optionally be included between the transmembrane domain and any remaining intracellular domain. Such optional linkers or spacers are described herein. In addition, such linkers or spacers can be derived from a heterologous sequence. For example, as described above, a transmembrane domain derived from a heterologous polypeptide can optionally include additional sequences at the N-terminus and/or C-terminus derived from the heterologous polypeptide. Such additional sequences can function as a linker or spacer.

In one embodiment, as described above, a dominant negative form can lack any signaling domain carboxy-terminal to the transmembrane domain of the dominant negative form (i.e., the dominant negative form can lack an intracellular signaling domain).

In a different specific embodiment, a dominant negative form of the invention can optionally further comprise a fusion to a co-stimulatory signaling domain, wherein the co-stimulatory signaling domain is carboxy-terminal to the transmembrane domain of the dominant negative form. Such a dominant negative form is also referred to herein as a “switch receptor.” Such a dominant negative form, or switch receptor, comprises at least a ligand binding domain of the extracellular region of an inhibitor of a cell-mediated immune response of the cell, such as an immune checkpoint inhibitor, fused to a transmembrane domain, fused to a co-stimulatory domain (i.e., cytoplasmic signaling domain) of an immunostimulatory molecule, thereby switching the activity upon ligand binding from inhibitory of the cell immune activity to stimulatory of the cell immune activity (see e.g., Liu et al., Cancer Res. 76:1578-1590 (2016)). A dominant negative form further comprising a fusion to a co-stimulatory domain (i.e., switch receptor) also functions as a dominant negative form in such a construct since the signaling domain of the immune checkpoint inhibitor has been deleted. In one embodiment, a dominant negative form further comprising a fusion to a co-stimulatory signaling domain is expressed in an immunostimulatory cell. In one embodiment, a dominant negative form further comprising a fusion to a co-stimulatory signaling domain is expressed in an immunoinhibitory cell. In another embodiment, a dominant negative form further comprising a fusion to a co-stimulatory signaling domain is co-expressed with a CAR in an immunostimulatory cell. In another embodiment, a dominant negative form further comprising a fusion to a co-stimulatory signaling domain is co-expressed with a CAR in an immunostimulatory cell.

A co-stimulatory signaling domain in a dominant negative form fusion polypeptide can be derived, for example, from a cytoplasmic signaling domain of a receptor such as the co-stimulatory molecules described herein for use in a CAR, including but not limited to a 4-1BB polypeptide, a CD28 polypeptide, an OX40 polypeptide, an ICOS polypeptide, a DAP10 polypeptide, and a 2B4 polypeptide. In a dominant negative form comprising a fusion to a co-stimulatory signaling domain, the transmembrane domain can be derived from the polypeptide from which the co-stimulatory domain is derived, from the polypeptide from which the extracellular ligand binding domain of dominant negative form is derived, or it can be a transmembrane domain from another polypeptide, similar to the description herein of the transmembrane domains that can be utilized to generate a CAR or dominant negative form.

In one embodiment, the invention provides an immunostimulatory cell that recombinantly expresses a dominant negative form, wherein the dominant negative form further comprises a fusion to a co-stimulatory signaling domain, wherein the co-stimulatory signaling domain is fused carboxy-terminal to the transmembrane domain of the dominant negative form. In certain embodiments of the invention, the cell or population of the invention recombinantly expresses a dominant negative form of an inhibitor of a cell-mediated immune response of the cell, wherein the dominant negative form further comprises a co-stimulatory signaling domain, wherein the co-stimulatory signaling domain is fused to the transmembrane domain of the dominant negative form (which in turn is fused to the at least a portion of the extracellular domain of an immune checkpoint inhibitor containing the ligand binding region of the dominant negative form). Such cells optionally can co-express a dominant negative form that lacks an intracellular signaling domain. Such cells can be used to treat a cancer or pathogen infection (such as viral infection) as disclosed herein. The invention provides for recombinant expression by an immunostimulatory cell of a switch receptor (i.e., a dominant negative form further comprising a co-stimulatory signaling domain), which switch receptor comprises (i) at least the extracellular ligand binding domain of an immune checkpoint inhibitor, (ii) a transmembrane domain, and (iii) a co-stimulatory signaling domain. Such recombinant cells optionally can co-express a dominant negative form that lacks an intracellular signaling domain. The invention also provides for recombinant expression by an immunostimulatory cell of both a CAR and a dominant negative form, which dominant negative form further comprises a fusion to a co-stimulatory signaling domain (switch receptor), which dominant negative form comprises (i) at least the extracellular ligand binding domain of an immune checkpoint inhibitor, (ii) a transmembrane domain, and (iii) a co-stimulatory signaling domain. Such cells optionally can co-express a dominant negative form that lacks an intracellular signaling domain. It is understood that, in such immunostimulatory cells co-expressing a CAR, and a dominant negative form further comprising a fusion to a co-stimulatory signaling domain (switch receptor), and optionally a dominant negative form lacking an intracellular signaling domain, the CAR binds to an antigen of the cancer or pathogen infection as being treated, i.e., the same pathogen of the pathogen infection. In one embodiment of cells co-expressing a CAR and a dominant negative form comprising a fusion to a co-stimulatory signaling domain, the co-stimulatory signaling domain of the dominant negative form is different from the co-stimulatory signaling domain of the CAR. In a particular embodiment, the co-stimulatory signaling domain of the dominant negative form is the intracellular signaling domain of 4-1BB. In another particular embodiment, in an immunostimulatory cell co-expressing a CAR and a dominant negative form that further comprises a fusion to a co-stimulatory signaling domain, the co-stimulatory signaling domain of the CAR is the intracellular signaling domain of CD28. In another particular embodiment, the invention provides an immunostimulatory cell co-expressing a CAR and a dominant negative form that further comprises a fusion to a co-stimulatory signaling domain, and optionally co-expresses a dominant negative form that lacks an intracellular signaling domain, where the co-stimulatory signaling domain of the dominant negative form is the intracellular signaling domain of 4-1BB and the co-stimulatory signaling domain of the CAR is the intracellular signaling domain of CD28.

Exemplary dominant negative forms of immune checkpoint inhibitors are described below in more detail. Dominant negative forms consisting essentially of the described sequences are also envisioned.

PD-1. Programmed cell death protein 1 (PD-1) is a negative immune regulator of activated T cells upon engagement with its corresponding ligands, PD-L1 and PD-L2, expressed on endogenous macrophages and dendritic cells. PD-1 is a type I membrane protein of 268 amino acids. PD-1 has two ligands, PD-L1 and PD-L2, which are members of the B7 family. The protein's structure comprises an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif. PD-1 negatively regulates TCR signals. SHP-1 and SHP-2 phosphatases bind to the cytoplasmic tail of PD-1 upon ligand binding. Upregulation of PD-L1 is one mechanism tumor cells use to evade the host immune system. In pre-clinical and clinical trials, PD-1 blockade by antagonistic antibodies induced anti-tumor responses mediated through the host endogenous immune system.

A PD-1 polypeptide can have an amino acid corresponding to GenBank No. NP_005009.2 (GI:167857792), as provided below, or fragments thereof. See GenBank NP_005009.2 for reference to domains within PD-1, for example, signal peptide, amino acids 1 to 20; extracellular domain, amino acids 21 to 170; transmembrane domain, amino acids 171 to 191; intracellular domain, amino acids 192 to 288. It is understood that an “PD-1 nucleic acid molecule” refers to a polynucleotide encoding an PD-1 polypeptide.

| (NP_005009.2; SEQ ID NO: 33) | |
|---|---|
| 1 | MQIPQAPWPV VWAVLQLGWR PGWFLDSPDR PWNPPTFSPA LLVVTEGDNA TFTCSFSNTS |
| 61 | ESFVLNWYRM SPSNQTDKLA AFPEDRSQPG QDCRFRVTQL PNGRDFHMSV VRARRNDSGT |
| 121 | YLCGAISLAP KAQIKESLRA ELRVTERRAE VPTAHPSPSP RPAGQFQTLV VGVVGGLLGS |
| 181 | LVLLVWVLAV ICSRAARGTI GARRTGQPLK EDPSAVPVFS VDYGELDFQW REKTPEPPVP |
| 241 | CVPEQTEYAT IVFPSGMGTS SPARRGSADG PRSAQPLRPE DGHCSWPL |

In one embodiment, the inhibitor of a cell-mediated immune response is a PD-1 dominant negative form (dominant negative form). In one embodiment, the PD-1 dominant negative form comprises the extracellular ligand binding domain of PD-1. In one embodiment, the PD-1 dominant negative form comprises the extracellular ligand binding domain of PD-1 and a transmembrane domain (e.g., mature form). In another embodiment, the PD-1 dominant negative form comprises the extracellular ligand binding domain of PD-1, a transmembrane domain and a signal peptide (e.g., precursor form). The invention also provides encoding polypeptides and nucleic acids of the PD-1 dominant negative forms of the invention. In a particular embodiment, the PD-1 extracellular ligand binding domain is fused to one or more heterologous polypeptide sequences, that is, the PD-1 dominant negative form is a chimeric sequence. For example, the PD-1 extracellular ligand binding domain can be fused at its N-terminus to a signal peptide that is optionally a heterologous signal peptide, including various signal peptides described herein. In addition, a PD-1 dominant negative form can comprise a transmembrane domain that is optionally a heterologous transmembrane domain, including any of various transmembrane domains described herein. Although the PD-1 dominant negative form exemplified in the Example herein comprises heterologous sequences fused to the extracellular domain of PD-1, it is understood that a PD-1 dominant negative form can comprise PD-1 sequence only.

In one embodiment, the inhibitor of a cell-mediated immune response is a PD-1 dominant negative form that comprises the extracellular domain, or a ligand binding portion thereof, of PD-1, for example, amino acids 21 to 170 corresponding to the extracellular domain of PD-1 (GenBank NP_005009.2; SEQ ID NO:33). A cell expressing such a PD-1 dominant negative form should lack the ability or have reduced ability to signal in a PD-1 immune checkpoint pathway. In one embodiment, a PD-1 dominant negative form is a deletion mutant having a deletion of the intracellular domain, for example, amino acids 192 to 288 of PD-1 (GenBank NP_005009.2; SEQ ID NO:33), or a portion thereof, such that intracellular signaling of the immune checkpoint pathway mediated by PD-1 is reduced or inhibited. Additional embodiments of a dominant negative form of PD-1 are described below.

In one embodiment, a PD-1 dominant negative form comprises an amino acid sequence comprising the extracellular domain of PD-1 fused to the transmembrane and hinge domains of CD8. In one embodiment, a PD-1 dominant negative form comprises amino acids 21 to 165 of a PD-1 sequence (NP_005009.2; SEQ ID NO:33). Such a PD-1 dominant negative form comprises the extracellular domain of PD-1. In another embodiment, the inhibitor of a cell-mediated immune response is a PD-1 dominant negative form comprising amino acids 1 to 165 (precursor form) or amino acids 21 to 165 (mature form) of a PD-1 sequence (NP_005009.2; SEQ ID NO:33). Such a dominant negative form comprises the signal peptide of PD-1, amino acids 1 to 20, and extracellular domain amino acids 21 to 165, whereas the mature form lacks the signal peptide. In one embodiment, a PD-1 dominant negative form comprises amino acids 21 to 151 of a PD-1 sequence (NP_005009.2; SEQ ID NO:33). In another embodiment, the inhibitor of a cell-mediated immune response is a PD-1 dominant negative form comprising amino acids 1 to 151 (precursor form) or amino acids 21 to 151 (mature form) of a PD-1 sequence (NP_005009.2; SEQ ID NO:33). Optionally, a PD-1 dominant negative form comprises an extracellular ligand binding domain starting at amino acid 21 through an amino acid between amino acids 151 to 165 of a PD-1 sequence (NP_005009.2; SEQ ID NO:33). In another embodiment, a PD-1 dominant negative form comprises the transmembrane domain of CD8, amino acids 183 to 203 of a CD8 sequence (NP_001139345.1; SEQ ID NO:11). Such an embodiment is representative of a chimeric dominant negative form comprising a transmembrane domain from a different (heterologous) polypeptide. As described above, a dominant negative form comprising a heterologous domain such as a transmembrane domain can optionally include additional sequence from the heterologous polypeptide. In one such embodiment, a dominant negative form is provided that comprises additional sequence from the heterologous polypeptide N-terminal of the transmembrane domain. In one embodiment, the dominant negative form comprises the hinge domain of CD8. In a particular embodiment, the heterologous sequence comprises additional N-terminal sequence of amino acids 137 to 182, or optionally starting at amino acids 138 or 139, of a CD8 sequence (NP_001139345.1; SEQ ID NO:11). In another embodiment, a dominant negative form is provided that comprises additional sequence from the heterologous polypeptide C-terminal of the transmembrane domain. In a particular embodiment, the heterologous sequence comprises additional C-terminal sequence from amino acids 204 to 209 of a CD8 sequence (NP_001139345.1; SEQ ID NO:11). In one embodiment, the PD-1 dominant negative form comprises the transmembrane domain of CD8, amino acids 183 to 203, optionally a hinge domain comprising amino acids 137 to 182 (or optionally starting at amino acids 138 or 139), and/or additional C-terminal sequence comprising amino acids 204 to 209. In a particular embodiment of the invention, a PD-1 dominant negative form is provided that comprises amino acids 1 to 165 of a PD-1 sequence (NP_005009.2; SEQ ID NO:33), and amino acids 137 to 209, optionally starting at amino acids 138 or 139, of a CD8 sequence (NP_001139345.1; SEQ ID NO:11).

In a further particular embodiment, the inhibitor of a cell-mediated immune response is a PD-1 dominant negative form comprising the sequence provided below, where the underlined sequence is derived from PD-1 and the italicized sequence is derived from CD8.

(SEQ ID NO: 43)

MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQAAAPTTTPAPRPPTPAPTIASQPLSLRPEAC
RPAAGGAVHTRGLDFACDIYIWAPLAGTCGVLLLSLVITLYCNHRRIQ

In an additional embodiment, a dominant negative form of the invention optionally comprises a P2A sequence, which provides for optional co-expression of a reporter molecule. P2A is a sequence used for bicistronic or multicistronic expression of protein sequences (see Szymczak et al., Expert Opin. Biol. Therapy 5(5):627-638 (2005)). An exemplary P2A sequence is GSGATNFSLLKQAGDVEENPGPM (SEQ ID NO:44). In a further embodiment, a dominant negative form of the invention is co-expressed with a reporter protein. In a particular embodiment, the reporter protein is mCherry fluorescent protein. In a particular embodiment, the mCherry polypeptide sequence is as provided below. It is understood that mCherry is merely exemplary and that any desired reporter molecule, such as a fluorescent protein can be included as a reporter, as described herein.

(SEQ ID NO: 45)

MVSKGEEDNMAIIKEFMRFKVHMEGSVNGHEFEIEGEGEGRPYEGTQTA
KLKVTKGGPLPFAWDILSPQFMYGSKAYVKHPADIPDYLKLSFPEGFKW
ERVMNFEDGGVVTVTQDSSLQDGEFIYKVKLRGTNFPSDGPVMQKKTMG
WEASSERMYPEDGALKGEIKQRLKLKDGGHYDAEVKTTYKAKKPVQLPG
AYNVNIKLDITSHNEDYTIVEQYERAEGRHSTGGMDELYK

In a further particular embodiment, a PD-1 dominant negative form is expressed as a polypeptide construct as provided below, where the underlined sequence is derived from PD-1, the italicized sequence is derived from CD8, the P2A sequence is double underlined, and the mCherry sequence is underlined and italicized.

(SEQ ID NO: 46)

MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQAAAPTTTPAPRPPTPAPTIASQPLSLRPEAC
RPAAGGAVHTRGLDFACDIYIWAPLAGTCGVLLLSLVITLYCNHRRIQG
SGATNFSLLKQAGDVEENPGPMVSKGEEDNMAIIKEFMRFKVHMEGSVN
GHEFEIEGEGEGRPYEGTQTAKLKVTKGGPLPFAWDILSPQFMYGSKAY
VKHPADIPDYLKLSFPEGFKWERVMNFEDGGVVTVTQDSSLQDGEFIYK
VKLRGTNFPSDGPVMQKKTMGWEASSERMYPEDGALKGEIKQRLKLKDG
GHYDAEVKTTYKAKKPVQLPGAYNVNIKLDITSHNEDYTIVEQYERAEG
RHSTGGMDELYK

In a particular embodiment, a nucleic acid encoding a dominant negative form of PD-1 is provided below, where the underlined sequence encodes amino acids derived from PD-1 dominant negative form, the italicized sequence encodes amino acids derived from CD8, the P2A encoding sequence is double underlined, the mCherry encoding sequence is underlined and italicized, a Kozak sequence is bolded with a dashed underline, and restriction sites Age I and Xho I are underlined with a dotted line at the 5′ and 3′ ends, respectively.

(SEQ ID NO: 47)

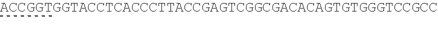

GACACCAGACTAAGAACCTAGAACCTCGCTGGAAAGGACCTTACACAGTC
CTGCTGACCACCCCCACCGCCCTCAAAGTAGACGGCATCGCAGCTTGGAT
ACACGCCGCCCACGTGAAGGCTGCCGACCCCGGGGGTGGACCATCCTCTA

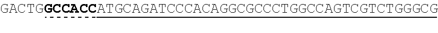

GTGCTACAACTGGGCTGGCGGCCAGGATGGTTCTTAGACTCCCCAGACAG
GCCCTGGAACCCCCCCACCTTCTCCCCAGCCCTGCTCGTGGTGACCGAAG
GGGACAACGCCACCTTCACCTGCAGCTTCTCCAACACATCGGAGAGCTTC
GTGCTAAACTGGTACCGCATGAGCCCCAGCAACCAGACGGACAAGCTGGC
CGCTTTCCCCGAGGACCGCAGCCAGCCCGGCCAGGACTGCCGCTTCCGTG
TCACACAACTGCCCAACGGGCGTGACTTCCACATGAGCGTGGTCAGGGCC
CGGCGCAATGACAGCGGCACCTACCTCTGTGGGGCCATCTCCCTGGCCCC
CAAGGCGCAGATCAAAGAGAGCCTGCGGGCAGAGCTCAGGGTGACAGAGA
GAAGGGCAGAAGTGCCCACAGCCCACCCCAGCCCCTCACCCAGGCCAGCC
GGCCAGGCGGCCGCACCCACCACGACGCCAGCGCCGCGACCACCAACCCC
GGCGCCCACGATCGCGTCGCAGCCCCTGTCCCTGCGCCCAGAGGCGTGCC
GGCCAGCGGCGGGGGGCGCAGTGCACACGAGGGGGCTGGACTTCGCCTGT
GATATCTACATCTGGGCGCCCCTGGCCGGGACTTGTGGGGTCCTTCTCCT
GTCACTGGTTATCACCCTTTACTGCAACCACAGGCGGATCCAAGGATCTG
GAGCAACAAACTTCTCACTACTCAAACAAGCAGGTGACGTGGAGGAGAAT
CCCGGCCCCATGGTGAGCAAGGGCGAGGAGGATAACATGGCCATCATCAA
GGAGTTCATGCGCTTCAAGGTGCACATGGAGGGCTCCGTGAACGGCCACG
AGTTCGAGATCGAGGGCGAGGGCGAGGGCCGCCCCTACGAGGGCACCCAG
ACCGCCAAGCTGAAGGTGACCAAGGGTGGCCCCCTGCCCTTCGCCTGGGA
CATCCTGTCCCCTCAGTTCATGTACGGCTCCAAGGCCTACGTGAAGCACC
CCGCCGACATCCCCGACTACTTGAAGCTGTCCTTCCCCGAGGGCTTCAAG
TGGGAGCGCGTGATGAACTTCGAGGACGGCGGCGTGGTGACCGTGACCCA
GGACTCCTCCCTGCAGGACGGCGAGTTCATCTACAAGGTGAAGCTGCGCG
GCACCAACTTCCCCTCCGACGGCCCCGTAATGCAGAAGAAGACCATGGGC
TGGGAGGCCTCCTCCGAGCGGATGTACCCCGAGGACGGCGCCCTGAAGGG
CGAGATCAAGCAGAGGCTGAAGCTGAAGGACGGCGGCCACTACGACGCTG
AGGTCAAGACCACCTACAAGGCCAAGAAGCCCGTGCAGCTGCCCGGCGCC
TACAACGTCAACATCAAGTTGGACATCACCTCCCACAACGAGGACTACAC
CATCGTGGAACAGTACGAACGCGCCGAGGGCCGCCACTCCACCGGCGGCA

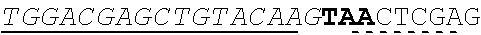

CTLA-4. Cytotoxic T-lymphocyte-associated protein 4 (CTLA-4) is an inhibitory receptor expressed by activated T cells, which when engaged by its corresponding ligands (CD80 and CD86; B7-1 and B7-2, respectively), mediates activated T cell inhibition or anergy. In both preclinical and clinical studies, CTLA-4 blockade by systemic antibody infusion enhanced the endogenous anti-tumor response albeit, in the clinical setting, with significant unforeseen toxicities. CTLA-4 contains an extracellular V domain, a transmembrane domain, and a cytoplasmic tail. Alternate splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM (SEQ ID NO:49) motif able to bind PI3K, PP2A and SHP-2 and one proline-rich motif able to bind SH3 containing proteins. One role of CTLA-4 in inhibiting T cell responses seems to be directly via SHP-2 and PP2A dephosphorylation of TCR-proximal signaling proteins such as CD3 and LAT. CTLA-4 can also affect signaling indirectly via competing with CD28 for CD80/86 binding. CTLA-4 has also been shown to bind and/or interact with PI3K, CD80, AP2M1, and PPP2R5A.

A CTLA-4 polypeptide can have an amino acid sequence corresponding to GenBank No. AAH69566.1 (GI:46854814) or NP_005205.2 (GI:21361212), sequence as provided below, or fragments thereof. See GenBank NP_005205.2 for reference to domains within CTLA-4, for example, signal peptide, amino acids 1 to 35; extracellular domain, amino acids 36 to 161; transmembrane domain, amino acids 162 to 182; intracellular domain, amino acids 183 to 223. It is understood that a “CTLA-4 nucleic acid molecule” refers to a polynucleotide encoding a CTLA-4 polypeptide.

| (NP_005205.2; SEQ ID NO: 34) | |
|---|---|
| 1 | MACLGFQRHK AQLNLATRTW PCTLLFFLLF IPVFCKAMHV AQPAVVLASS RGIASFVCEY |
| 61 | ASPGKATEVR VTVLRQADSQ VTEVCAATYM MGNELTFLDD SICTGTSSGN QVNLTIQGLR |
| 121 | AMDTGLYICK VELMYPPPYY LGIGNGTQIY VIDPEPCPDS DFLLWILAAV SSGLFFYSFL |
| 181 | LTAVSLSKML KKRSPLTTGV YVKMPPTEPE CEKQFQPYFI PIN |

In one embodiment, the inhibitor of a cell-mediated immune response is a CTLA-4 dominant negative form. In one embodiment, the CTLA-4 dominant negative form comprises the extracellular ligand binding domain of CTLA-4. In one embodiment, the CTLA-4 dominant negative form comprises the extracellular ligand binding domain of CTLA-4 and a transmembrane domain (e.g., mature form). In another embodiment, the CTLA-4 dominant negative form comprises the extracellular ligand binding domain of CTLA-4, a transmembrane domain and a signal peptide (e.g., precursor form). The invention also provides encoding polypeptides and nucleic acids of the CTLA-4 dominant negative forms of the invention. In a particular embodiment, the CTLA-4 extracellular ligand binding domain is fused to one or more heterologous polypeptide sequences, that is, the CTLA-4 dominant negative form is chimeric. For example, the CTLA-4 extracellular ligand binding domain can be fused at its N-terminus to a signal peptide that is optionally a heterologous signal peptide, including various signal peptides described herein. In addition, a CTLA-4 dominant negative form can comprise a transmembrane domain that is optionally a heterologous transmembrane domain, including any of various transmembrane domains described herein.

In an embodiment of the invention, the CTLA-4 dominant negative form can comprise the extracellular domain, or a ligand binding portion thereof, of CTLA-4, for example, amino acids 36 to 161 corresponding to the extracellular domain of CTLA-4 (GenBank NP_005205.2; SEQ ID NO:34). A cell expressing such a CTLA-4 dominant negative form should lack the ability or have reduced ability to signal in a CTLA-4 immune checkpoint pathway. In one embodiment, a CTLA-4 dominant negative form is a deletion mutant having a deletion of the intracellular domain, for example, amino acids 183 to 223 of CTLA-4 (GenBank NP_005205.2; SEQ ID NO:34), or a portion thereof, such that intracellular signaling of the immune checkpoint pathway mediated by CTLA-4 is reduced or inhibited.

BTLA. B- and T-lymphocyte attenuator (BTLA) expression is induced during activation of T cells, and BTLA remains expressed on Th1 cells but not Th2 cells. BTLA interacts with a B7 homolog, B7H4. BTLA displays T-Cell inhibition via interaction with tumor necrosis family receptors (TNF-R), not just the B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses. BTLA activation has been shown to inhibit the function of human CD8⁺ cancer-specific T cells. BTLA has also been designated as CD272 (cluster of differentiation 272).

A BTLA polypeptide can have an amino acid sequence corresponding to GenBank No. AAP44003.1 (GI:31880027) or NP_861445.3 (GI:145580621), sequence provided below, or fragments thereof. See GenBank NP_861445.3 for reference to domains within BTLA, for example, signal peptide, amino acids 1 to 30; extracellular domain, amino acids 31 to 157; transmembrane domain, amino acids 158 to 178; intracellular domain, amino acids 179 to 289. It is understood that a “BTLA nucleic acid molecule” refers to a polynucleotide encoding a BTLA polypeptide.

| (NP_861445.3; SEQ ID NO: 35) | |
|---|---|
| 1 | MKTLPAMLGT GKLFWVFFLI PYLDIWNIHG KESCDVQLYI KRQSEHSILA GDPFELECPV |
| 61 | KYCANRPHVT WCKLNGTTCV KLEDRQTSWK EEKNISFFIL HFEPVLPNDN GSYRCSANFQ |
| 121 | SNLIESHSTT LYVTDVKSAS ERPSKDEMAS RPWLLYSLLP LGGLPLLITT CFCLFCCLRR |
| 181 | HQGKQNELSD TAGREINLVD AHLKSEQTEA STRQNSQVLL SETGIYDNDP DLCFRMQEGS |
| 241 | EVYSNPCLEE NKPGIVYASL NHSVIGPNSR LARNVKEAPT EYASICVRS |

In one embodiment, the inhibitor of a cell-mediated immune response is a BTLA dominant negative form. In one embodiment, the BTLA dominant negative form comprises the extracellular ligand binding domain of BTLA. In one embodiment, the BTLA dominant negative form comprises the extracellular ligand binding domain of BTLA and a transmembrane domain (e.g., mature form). In another embodiment, the BTLA dominant negative form comprises the extracellular ligand binding domain of BTLA, a transmembrane domain and a signal peptide (e.g., precursor form). The invention also provides encoding polypeptides and nucleic acids of the BTLA dominant negative forms of the invention. In a particular embodiment, the BTLA extracellular ligand binding domain is fused to one or more heterologous polypeptide sequences, that is, the BTLA dominant negative form is chimeric. For example, the BTLA extracellular ligand binding domain can be fused at its N-terminus to a signal peptide that is optionally a heterologous signal peptide, including various signal peptides described herein. In addition, a BTLA dominant negative form can comprise a transmembrane domain that is optionally a heterologous transmembrane domain, including any of various transmembrane domains described herein.

In an embodiment of the invention, the BTLA dominant negative form can comprise the extracellular domain, or a ligand binding portion thereof, of BTLA, for example, amino acids 31 to 157 corresponding to the extracellular domain of BTLA (GenBank NP_861445.3; SEQ ID NO:35). A cell expressing such a BTLA dominant negative form should lack the ability or have reduced ability to signal in a BTLA immune checkpoint pathway. In one embodiment, a BTLA dominant negative form is a deletion mutant having a deletion of the intracellular domain, for example, amino acids 179 to 289 of BTLA (GenBank NP_861445.3; SEQ ID NO:35), or a portion thereof, such that intracellular signaling of the immune checkpoint pathway mediated by BTLA is reduced or inhibited.

TIM-3. T cell immunoglobulin mucin-3 (TIM-3), also referred to as hepatitis A virus cellular receptor 2 precursor, is a Thl-specific cell surface protein that regulates macrophage activation. TIM-3 was first identified as a molecule selectively expressed on IFN-γ-producing CD4+ T helper 1 (Th1) and CD8+ T cytotoxic 1 (Te1) T cells. TIM-3 possess an N-terminal Ig domain of the V type, followed by a mucin domain.

A TIM-3 polypeptide can have an amino acid sequence corresponding to GenBank No. NP_116171.3 (GI:49574534), sequence provided below, or fragments thereof. See GenBank NP_116171.3 for reference to domains within TIM-3, for example, signal peptide, amino acids 1 to 21; extracellular domain, amino acids 22 to 202; transmembrane domain, amino acids 203 to 223; intracellular domain, amino acids 224 to 301. It is understood that a “TIM-3 nucleic acid molecule” refers to a polynucleotide encoding a TIM-3 polypeptide.

| (NP_116171.3; SEQ ID NO: 36) | |
|---|---|
| 1 | MFSHLPFDCV LLLLLLLLTR SSEVEYRAEV GQNAYLPCFY TPAAPGNLVP VCWGKGACPV |
| 61 | FECGNVVLRT DERDVNYWTS RYWLNGDFRK GDVSLTIENV TLADSGIYCC RIQIPGIMND |
| 121 | EKFNLKLVIK PAKVTPAPTR QRDFTAAFPR MLTTRGHGPA ETQTLGSLPD INLTQISTLA |
| 181 | NELRDSRLAN DLRDSGATIR IGIYIGAGIC AGLALALIFG ALIFKWYSHS KEKIQNLSLI |
| 241 | SLANLPPSGL ANAVAEGIRS EENIYTIEEN VYEVEEPNEY YCYVSSRQQP SQPLGCRFAM |
| 301 | P |

In one embodiment, the inhibitor of a cell-mediated immune response is a TIM-3 dominant negative form. In one embodiment, the TIM-3 dominant negative form comprises the extracellular ligand binding domain of TIM-3. In one embodiment, the TIM-3 dominant negative form comprises the extracellular ligand binding domain of TIM-3 and a transmembrane domain (e.g., mature form). In another embodiment, the TIM-3 dominant negative form comprises the extracellular ligand binding domain of TIM-3, a transmembrane domain and a signal peptide (e.g., precursor form). The invention also provides encoding polypeptides and nucleic acids of the TIM-3 dominant negative forms of the invention. In a particular embodiment, the TIM-3 extracellular ligand binding domain is fused to one or more heterologous polypeptide sequences, that is, the TIM-3 dominant negative form is chimeric. For example, the TIM-3 extracellular ligand binding domain can be fused at its N-terminus to a signal peptide that is optionally a heterologous signal peptide, including various signal peptides described herein. In addition, a TIM-3 dominant negative form can comprise a transmembrane domain that is optionally a heterologous transmembrane domain, including any of various transmembrane domains described herein.

In an embodiment of the invention, the TIM-3 dominant negative form can comprise the extracellular domain, or a ligand binding portion thereof, of TIM-3, for example, amino acids 22 to 202 corresponding to the extracellular domain of TIM-3 (GenBank NP_116171.3; SEQ ID NO:36). A cell expressing such a TIM-3 dominant negative form should lack the ability or have reduced ability to signal in a TIM-3 immune checkpoint pathway. In one embodiment, a TIM-3 dominant negative form is a deletion mutant having a deletion of the intracellular domain, for example, amino acids 224 to 301 of TIM-3 (GenBank NP_116171.3; SEQ ID NO:36), or a portion thereof, such that intracellular signaling of the immune checkpoint pathway mediated by TIM-3 is reduced or inhibited.

LAG-3. Lymphocyte-activation protein 3 (LAG-3) is a negative immune regulator of immune cells. LAG-3 belongs to the immunoglobulin (Ig) superfamily and contains 4 extracellular Ig-like domains. The LAG3 gene contains 8 exons. The sequence data, exon/intron organization, and chromosomal localization all indicate a close relationship of LAG-3 to CD4. LAG-3 has also been designated CD223 (cluster of differentiation 223).

A LAG-3 polypeptide can have an amino acid sequence corresponding to GenBank No. CAA36243.3 (GI:15617341) or NP_002277.4 (GI:167614500), sequence provided below, or fragments thereof. See GenBank NP_002277.4 for reference to domains within LAG-3, for example, signal peptide, amino acids 1 to 22; extracellular domain, amino acids 23 to 450; transmembrane domain, amino acids 451 to 471; intracellular domain, amino acids 472 to 525. It is understood that a “LAG-3 nucleic acid molecule” refers to a polynucleotide encoding a LAG-3 polypeptide.

| (NP_002277.4; SEQ ID NO: 37) | |
|---|---|
| 1 | MWEAQFLGLL FLQPLWVAPV KPLQPGAEVP VVWAQEGAPA QLPCSPTIPL QDLSLLRRAG |
| 61 | VTWQHQPDSG PPAAAPGHPL APGPHPAAPS SWGPRPRRYT VLSVGPGGLR SGRLPLQPRV |
| 121 | QLDERGRQRG DFSLWLRPAR RADAGEYRAA VHLRDRALSC RLRLRLGQAS MTASPPGSLR |
| 181 | ASDWVILNCS FSRPDRPASV HWFRNRGQGR VPVRESPHHH LAESFLFLPQ VSPMDSGPWG |
| 241 | CILTYRDGFN VSIMYNLTVL GLEPPTPLTV YAGAGSRVGL PCRLPAGVGT RSFLTAKWTP |
| 301 | PGGGPDLLVT GDNGDFTLRL EDVSQAQAGT YTCHIHLQEQ QLNATVTLAI ITVTPKSFGS |
| 361 | PGSLGKLLCE VTPVSGQERF VWSSLDTPSQ RSFSGPWLEA QEAQLLSQPW QCQLYQGERL |
| 421 | LGAAVYFTEL SSPGAQRSGR APGALPAGHL LLFLILGVLS LLLLVTGAFG FHLWRRQWRP |
| 481 | RRFSALEQGI HPPQAQSKIE ELEQEPEPEP EPEPEPEPEP EPEQL |

In one embodiment, the inhibitor of a cell-mediated immune response is a LAG-3 dominant negative form. In one embodiment, the LAG-3 dominant negative form comprises the extracellular ligand binding domain of LAG-3. In one embodiment, the LAG-3 dominant negative form comprises the extracellular ligand binding domain of LAG-3 and a transmembrane domain (e.g., mature form). In another embodiment, the LAG-3 dominant negative form comprises the extracellular ligand binding domain of LAG-3, a transmembrane domain and a signal peptide (e.g., precursor form). The invention also provides encoding polypeptides and nucleic acids of the LAG-3 dominant negative forms of the invention. In a particular embodiment, the LAG-3 extracellular ligand binding domain is fused to one or more heterologous polypeptide sequences, that is, the LAG-3 dominant negative form is chimeric. For example, the LAG-3 extracellular ligand binding domain can be fused at its N-terminus to a signal peptide that is optionally a heterologous signal peptide, including various signal peptides described herein. In addition, a LAG-3 dominant negative form can comprise a transmembrane domain that is optionally a heterologous transmembrane domain, including any of various transmembrane domains described herein.

In an embodiment of the invention, the LAG-3 dominant negative form can comprise the extracellular domain, or a ligand binding portion thereof, of LAG-3, for example, amino acids 23 to 450 corresponding to the extracellular domain of LAG-3 (GenBank NP_002277.4; SEQ ID NO:37). A cell expressing such a LAG-3 dominant negative form should lack the ability or have reduced ability to signal in a LAG-3 immune checkpoint pathway. In one embodiment, a LAG-3 dominant negative form is a deletion mutant having a deletion of the intracellular domain, for example, amino acids 472 to 525 of LAG-3 (GenBank NP_002277.4; SEQ ID NO:37), or a portion thereof, such that intracellular signaling of the immune checkpoint pathway mediated by LAG-3 is reduced or inhibited.

TIGIT. T-cell immunoreceptor with Ig and ITIM domains (TIGIT) is a cell surface protein that suppresses T-cell activation. It belongs to the poliovirus receptor (PVR) family of immunoglobulin (Ig) proteins that share 3 conserved sequence motifs in their N-terminal Ig domains. A TIGIT polypeptide can have an amino acid sequence corresponding to GenBank No. NP_776160.2 (GI:256600228), sequence provided below, or fragments thereof. See GenBank NP_776160.2 for reference to domains within TIGIT, for example, signal peptide, amino acids 1 to 21; extracellular domain, amino acids 22 to 141; transmembrane domain, amino acids 142 to 162; intracellular domain, amino acids 163 to 244. It is understood that a “TIGIT nucleic acid molecule” refers to a polynucleotide encoding a TIGIT polypeptide.

| (NP_776160.2; SEQ ID NO: 38) | |
|---|---|
| 1 | MRWCLLLIWA QGLRQAPLAS GMMTGTIETT GNISAEKGGS IILQCHLSST TAQVTQVNWE |
| 61 | QQDQLLAICN ADLGWHISPS FKDRVAPGPG LGLTLQSLTV NDTGEYFCIY HTYPDGTYTG |
| 121 | RIFLEVLESS VAEHGARFQI PLLGAMAATL VVICTAVIVV VALTRKKKAL RIHSVEGDLR |
| 181 | RKSAGQEEWS PSAPSPPGSC VQAEAAPAGL CGEQRGEDCA ELHDYFNVLS YRSLGNCSFF |
| 241 | TETG |

In one embodiment, the inhibitor of a cell-mediated immune response is a TIGIT dominant negative form. In one embodiment, the TIGIT dominant negative form comprises the extracellular ligand binding domain of TIGIT. In one embodiment, the TIGIT dominant negative form comprises the extracellular ligand binding domain of TIGIT and a transmembrane domain (e.g., mature form). In another embodiment, the TIGIT dominant negative form comprises the extracellular ligand binding domain of TIGIT, a transmembrane domain and a signal peptide (e.g., precursor form). The invention also provides encoding polypeptides and nucleic acids of the TIGIT dominant negative forms of the invention. In a particular embodiment, the TIGIT extracellular ligand binding domain is fused to one or more heterologous polypeptide sequences, that is, the TIGIT dominant negative form is chimeric. For example, the TIGIT extracellular ligand binding domain can be fused at its N-terminus to a signal peptide that is optionally a heterologous signal peptide, including various signal peptides described herein. In addition, a TIGIT dominant negative form can comprise a transmembrane domain that is optionally a heterologous transmembrane domain, including any of various transmembrane domains described herein.

In an embodiment of the invention, the TIGIT dominant negative form can comprise the extracellular domain, or a ligand binding portion thereof, of TIGIT, for example, amino acids 22 to 141 corresponding to the extracellular domain of TIGIT (GenBank NP_776160.2; SEQ ID NO:38). A cell expressing such a TIGIT dominant negative form should lack the ability or have reduced ability to signal in a TIGIT immune checkpoint pathway. In one embodiment, a TIGIT dominant negative form is a deletion mutant having a deletion of the intracellular domain, for example, amino acids 163 to 244 of TIGIT (GenBank NP_776160.2; SEQ ID NO:38), or a portion thereof, such that intracellular signaling of the immune checkpoint pathway mediated by TIGIT is reduced or inhibited.

LAIR1. Leukocyte-associated immunoglobulin-like receptor 1 (LAIR1) is an inhibitory receptor that plays a constitutive negative regulatory role on cytolytic function of natural killer (NK) cells, B-cells and T-cells. LAIR exists in various isoforms. It is understood that any isoform can be selected to achieve a desired function. Exemplary isoforms include isoform a (NP_002278.2, GI:612407859), isoform b (NP_068352.2, GI:612407861), isoform c (NP_001275952.2, GI:612407867), isoform e (NP_001275954.2, GI:612407869), isoform f (NP_001275955.2, GI:612407863), isoform g (NP_001275956.2, GI:612407865), and the like. One exemplary isoform sequence, isoform a, is provided below. In one embodiment, a LAIR1 polypeptide can have an amino acid sequence corresponding to NP_002278.2, sequence provided below, or fragments thereof. See GenBank NP_002278.2 for reference to domains within LAIR1, for example, signal peptide, amino acids 1 to 21; extracellular domain, amino acids 22 to 165; transmembrane domain, amino acids 166 to 186; intracellular domain, amino acids 187 to 287. It is understood that a “LAIR1 nucleic acid molecule” refers to a polynucleotide encoding a LAIR1 polypeptide.

| (NP_002278.2; SEQ ID NO: 39) | |
|---|---|
| 1 | MSPHPTALLG LVLCLAQTIH TQEEDLPRPS ISAEPGTVIP LGSHVTFVCR GPVGVQTFRL |
| 61 | ERDSRSTYND TEDVSQASPS ESEARFRIDS VREGNAGLYR CIYYKPPKWS EQSDYLELLV |
| 121 | KESSGGPDSP DTEPGSSAGP TQRPSDNSHN EHAPASQGLK AEHLYILIGV SVVFLFCLLL |
| 181 | LVLFCLHRQN QIKQGPPRSK DEEQKPQQRP DLAVDVLERT ADKATVNGLP EKDRETDTSA |
| 241 | LAAGSSQEVT YAQLDHWALT QRTARAVSPQ STKPMAESIT YAAVARH |

In one embodiment, the inhibitor of a cell-mediated immune response is a LAIR1 dominant negative form. In one embodiment, the LAIR1 dominant negative form comprises the extracellular ligand binding domain of LAIR1. In one embodiment, the LAIR1 dominant negative form comprises the extracellular ligand binding domain of LAIR1 and a transmembrane domain (e.g., mature form). In another embodiment, the LAIR1 dominant negative form comprises the extracellular ligand binding domain of LAIR1, a transmembrane domain and a signal peptide (e.g., precursor form). The invention also provides encoding polypeptides and nucleic acids of the LAIR1 dominant negative forms of the invention. In a particular embodiment, the LAIR1 extracellular ligand binding domain is fused to one or more heterologous polypeptide sequences, that is, the LAIR1 dominant negative form is chimeric. For example, the LAIR1 extracellular ligand binding domain can be fused at its N-terminus to a signal peptide that is optionally a heterologous signal peptide, including various signal peptides described herein. In addition, a LAIR1 dominant negative form can comprise a transmembrane domain that is optionally a heterologous transmembrane domain, including any of various transmembrane domains described herein.

In an embodiment of the invention, the LAIR1 dominant negative form can comprise the extracellular domain, or a ligand binding portion thereof, of LAIR1, for example, amino acids 22 to 165 corresponding to the extracellular domain of LAIR1 (GenBank NP_002278.2; SEQ ID NO:39). A cell expressing such a LAIR1 dominant negative form should lack the ability or have reduced ability to signal in a LAIR1 immune checkpoint pathway. In one embodiment, a LAIR1 dominant negative form is a deletion mutant having a deletion of the intracellular domain, for example, amino acids 187 to 287 of LAIR1 (GenBank NP_002278.2; SEQ ID NO:39), or a portion thereof, such that intracellular signaling of the immune checkpoint pathway mediated by LAIR1 is reduced or inhibited.

2B4. Natural Killer Cell Receptor 2B4 (2B4) mediates non-MHC restricted cell killing on NK cells and subsets of T cells. The 2B4-S isoform is believed to be an activating receptor, and the 2B4- L isoform is believed to be a negative immune regulator of immune cells. 2B4 becomes engaged upon binding its high-affinity ligand, CD48. 2B4 contains a tyrosine-based switch motif, a molecular switch that allows the protein to associate with various phosphatases. 2B4 has also been designated CD244 (cluster of differentiation 244).

A 2B4 polypeptide can have an amino acid sequence corresponding to GenBank No. NP_001160135.1 (GI:262263435), sequence provided below, or fragments thereof. See GenBank NP_001160135.1 for reference to domains within 2B4, for example, signal peptide, amino acids 1 to 18; extracellular domain, amino acids 19 to 229; transmembrane domain, amino acids 230 to 250; intracellular domain, amino acids 251 to 370. It is understood that a “2B4 nucleic acid molecule” refers to a polynucleotide encoding a 2B4 polypeptide.

| (NP_001160135.1; SEQ ID NO: 40) | |
|---|---|
| 1 | MLGQVVTLIL LLLLKVYQGK GCQGSADHVV SISGVPLQLQ PNSIQTKVDS IAWKKLLPSQ |
| 61 | NGFHHILKWE NGSLPSNTSN DRFSFIVKNL SLLIKAAQQQ DSGLYCLEVT SISGKVQTAT |
| 121 | FQVFVFESLL PDKVEKPRLQ GQGKILDRGR CQVALSCLVS RDGNVSYAWY RGSKLIQTAG |
| 181 | NLTYLDEEVD INGTHTYTCN VSNPVSWESH TLNLTQDCQN AHQEFRFWPF LVIIVILSAL |
| 241 | FLGTLACFCV WRRKRKEKQS ETSPKEFLTI YEDVKDLKTR RNHEQEQTFP GGGSTIYSMI |
| 301 | QSQSSAPTSQ EPAYTLYSLI QPSRKSGSRK RNHSPSFNST IYEVIGKSQP KAQNPARLSR |
| 361 | KELENFDVYS |

In one embodiment, the inhibitor of a cell-mediated immune response is a 2B4 dominant negative form. In one embodiment, the 2B4 dominant negative form comprises the extracellular ligand binding domain of 2B4. In one embodiment, the 2B4 dominant negative form comprises the extracellular ligand binding domain of 2B4 and a transmembrane domain (e.g., mature form). In another embodiment, the 2B4 dominant negative form comprises the extracellular ligand binding domain of 2B4, a transmembrane domain and a signal peptide (e.g., precursor form). The invention also provides encoding polypeptides and nucleic acids of the 2B4 dominant negative forms of the invention. In a particular embodiment, the 2B4 extracellular ligand binding domain is fused to one or more heterologous polypeptide sequences, that is, the 2B4 dominant negative form is chimeric. For example, the 2B4 extracellular ligand binding domain can be fused at its N-terminus to a signal peptide that is optionally a heterologous signal peptide, including various signal peptides described herein. In addition, a 2B4 dominant negative form can comprise a transmembrane domain that is optionally a heterologous transmembrane domain, including any of various transmembrane domains described herein.

In an embodiment of the invention, the 2B4 dominant negative form can comprise the extracellular domain, or a ligand binding portion thereof, of 2B4, for example, amino acids 19 to 229 corresponding to the extracellular domain of 2B4 (GenBank NP_001160135.1; SEQ ID NO:40). A cell expressing such a 2B4 dominant negative form should lack the ability or have reduced ability to signal in a 2B4 immune checkpoint pathway. In one embodiment, a 2B4 dominant negative form is a deletion mutant having a deletion of the intracellular domain, for example, amino acids 251 to 370 of 2B4 (GenBank NP_001160135.1; SEQ ID NO:40), or a portion thereof, such that intracellular signaling of the immune checkpoint pathway mediated by 2B4 is reduced or inhibited.

CD160. CD160 is a glycosylphosphatidylinositol-anchored molecule containing a single IgV-like domain that binds to HVEM and functions as a co-inhibitory receptor on T cells. A CD160 polypeptide can have an amino acid sequence corresponding to GenBank NP_008984.1 (GI:5901910), sequence provided below, or fragments thereof. See GenBank NP_008984.1 for reference to domains within CD160, for example, signal peptide, amino acids 1 to 26; extracellular domain, amino acids 27 to 159. It is understood that a “CD160 nucleic acid molecule” refers to a polynucleotide encoding a CD160 polypeptide.

| (NP_008984.1; SEQ ID NO: 41) | |
|---|---|
| 1 | MLLEPGRGCC ALAILLAIVD IQSGGCINIT SSASQEGTRL NLICTVWHKK EEAEGFVVFL |
| 61 | CKDRSGDCSP ETSLKQLRLK RDPGIDGVGE ISSQLMFTIS QVTPLHSGTY QCCARSQKSG |
| 121 | IRLQGHFFSI LFTETGNYTV TGLKQRQHLE FSHNEGTLSS GFLQEKVWVM LVTSLVALQA |
| 181 | L |

In one embodiment, the inhibitor of a cell-mediated immune response is a CD160 dominant negative form. In one embodiment, the CD160 dominant negative form comprises the extracellular ligand binding domain of CD160. In one embodiment, the CD160 dominant negative form comprises the extracellular ligand binding domain of CD160 and a transmembrane domain (e.g., mature form). In another embodiment, the CD160 dominant negative form comprises the extracellular ligand binding domain of CD160, a transmembrane domain and a signal peptide (e.g., precursor form). The invention also provides encoding polypeptides and nucleic acids of the CD160 dominant negative forms of the invention. In a particular embodiment, the CD160 extracellular ligand binding domain is fused to one or more heterologous polypeptide sequences, that is, the CD160 dominant negative form is chimeric. For example, the CD160 extracellular ligand binding domain can be fused at its N-terminus to a signal peptide that is optionally a heterologous signal peptide, including various signal peptides described herein. In addition, a CD160 dominant negative form can comprise a transmembrane domain that is a heterologous transmembrane domain, including any of various transmembrane domains described herein.

In an embodiment of the invention, the CD160 dominant negative form can comprise the extracellular domain, or a ligand binding portion thereof, of CD160, for example, amino acids 27 to 159 corresponding to the extracellular domain of CD160 (GenBank NP_008984.1; SEQ ID NO:41). A cell expressing such a CD160 dominant negative form should lack the ability or have reduced ability to signal in an immune checkpoint pathway. In one embodiment, the CD160 dominant negative form comprises the extracellular domain of CD160, or a ligand binding portion thereof, and a transmembrane domain derived from a heterologous polypeptide, including but not limited to one of the transmembrane domains described herein. In one non-limiting embodiment, the CD160 dominant negative form comprises the transmembrane domain of CD8. In a cell expressing the CD160 dominant negative form, intracellular signaling of the immune checkpoint pathway mediated by CD160 should be reduced or inhibited.

TGF-62- 3 Receptor Type 2. TGF-β receptor type 2 binds to TGF-β and a type I receptor dimer forming a heterotetrameric complex with the ligand. A TGF-β receptor type 2 polypeptide can have an amino acid sequence corresponding to GenBank No. NP_001020018.1 (GI:67782326), sequence provided below, or fragments thereof. See GenBank NP_001020018.1 for reference to domains within TGF-β receptor type 2, for example, signal peptide, amino acids 1 to 22; extracellular domain, amino acids 23 to 191; transmembrane domain, amino acids 192 to 212; intracellular domain, amino acids 213 to 592 (see also annotation in UniProtKB - P37173). It is understood that a “TGF-β receptor type 2 nucleic acid molecule” refers to a polynucleotide encoding a TGF-β receptor type 2 polypeptide.

| (NP_001020018.1, SEQ ID NO: 42) | |
|---|---|
| 1 | MGRGLLRGLW PLHIVLWTRI ASTIPPHVQK SDVEMEAQKD EIICPSCNRT AHPLRHINND |
| 61 | MIVTDNNGAV KFPQLCKFCD VRFSTCDNQK SCMSNCSITS ICEKPQEVCV AVWRKNDENI |
| 121 | TLETVCHDPK LPYHDFILED AASPKCIMKE KKKPGETFFM CSCSSDECND NIIFSEEYNT |
| 181 | SNPDLLLVIF QVTGISLLPP LGVAISVIII FYCYRVNRQQ KLSSTWETGK TRKLMEFSEH |
| 241 | CAIILEDDRS DISSTCANNI NHNTELLPIE LDTLVGKGRF AEVYKAKLKQ NTSEQFETVA |
| 301 | VKIFPYEEYA SWKTEKDIFS DINLKHENIL QFLTAEERKT ELGKQYWLIT AFHAKGNLQE |
| 361 | YLTRHVISWE DLRKLGSSLA RGIAHLHSDH TPCGRPKMPI VHRDLKSSNI LVKNDLTCCL |
| 421 | CDFGLSLRLD PTLSVDDLAN SGQVGTARYM APEVLESRMN LENVESFKQT DVYSMALVLW |
| 481 | EMTSRCNAVG EVKDYEPPFG SKVREHPCVE SMKDNVLRDR GRPEIPSFWL NHQGIQMVCE |
| 541 | TLTECWDHDP EARLTAQCVA ERFSELEHLD RLSGRSCSEE KIPEDGSLNT TK |

In one embodiment, the inhibitor of a cell-mediated immune response is a TGFβ receptor dominant negative form. In one embodiment, the TGFβ receptor dominant negative form comprises the extracellular ligand binding domain of TGFβ receptor. In one embodiment, the TGFβ receptor dominant negative form comprises the extracellular ligand binding domain of TGFβ receptor and a transmembrane domain (e.g., mature form). In another embodiment, the TGFβ receptor dominant negative form comprises the extracellular ligand binding domain of TGFβ receptor, a transmembrane domain and a signal peptide (e.g., precursor form). The invention also provides encoding polypeptides and nucleic acids of the TGF-β receptor dominant negative forms of the invention. In a particular embodiment, the TGFβ receptor extracellular ligand binding domain is fused to one or more heterologous polypeptide sequences, that is, the TGFβ receptor dominant negative form is chimeric. For example, the TGFβ receptor extracellular ligand binding domain can be fused at its N-terminus to a signal peptide that is optionally a heterologous signal peptide, including various signal peptides described herein. In addition, a TGFβ receptor dominant negative form can comprise a transmembrane domain that is a heterologous transmembrane domain, including any of various transmembrane domains described herein.

TGFβ receptor dominant negative forms have been described previously (see, for example, Bottinger et al., EMBO 1 16:2621-2633 (1997), describing a dominant negative form comprising TGFβ receptor extracellular and transmembrane domains; Foster et al., J. Immunother. 31:500-505 (2008); Bollard et al., Blood 99:3179-3187 (2002); Wieser et al., Mol. Cell. Biol. 13:7239-7247 (1993)). In an embodiment of the invention, the TGFβ receptor dominant negative form can comprise the extracellular domain, or a ligand binding portion thereof, of TGFβ receptor, for example, amino acids 23 to 191 corresponding to the extracellular domain of TGFβ receptor (GenBank NP_001020018.1, SEQ ID NO:42). A cell expressing such a TGFβ receptor dominant negative form lacks the ability or has reduced ability to signal in the cell. In one embodiment, a TGFβ receptor dominant negative form is a deletion mutant having a deletion of the intracellular domain, for example, amino acids 213 to 592 of TGFβ receptor (GenBank NP_001020018.1, SEQ ID NO:42), or a portion thereof, such that intracellular signaling of mediated by TGFβ receptor is reduced or inhibited (see also Bottinger et al., EMBO 1 16:2621-2633 (1997); Foster et al., J. Immunother. 31:500-505 (2008); Bollard et al., Blood 99:3179-3187 (2002); Wieser et al., Mol. Cell. Biol. 13:7239-7247 (1993)).

It is understood that, optionally, a second dominant negative form of an inhibitor of a cell-mediated immune response, such as an immune checkpoint inhibitor, can be expressed in a cell of the invention. In this case, it can be desirable to inhibit more than one cell-mediated immune response in the same cell. Thus, a cell can express two or more dominant negative forms, each directed to a different inhibitor of a cell-mediated immune response, including those described above. For example, a dominant negative form of PD-1 can be co-expressed in a cell with a dominant negative form of TGF-β receptor, a dominant negative form of PD-1 can be co-expressed with a dominant negative form of CTLA-4, a CTLA-4 dominant negative form can be co-expressed with a dominant negative form of TGF-β receptor, and so forth, as desired, including combinations of any of the dominant negative forms described above.

In addition to immunostimulatory cells, the invention additionally provides a cell comprising one or more nucleotide sequences, transgenes, or vectors of the invention.

Additionally provided are recombinant cells expressing polypeptides, nucleic acids, transgenes, and/or vectors of the invention. Such a recombinant cell can be an immunostimulatory cell, such as a T cell. Such recombinant immunostimulatory cells are described in more detail above. Recombinant cells can be used for genetic manipulations prior to transduction of the immunostimulatory cells to be used therapeutically, such as generating constructs of the polypeptides and encoding nucleic acids of the invention, and/or for generating nucleic acid material for incorporation into a vector for expression in an immunostimulatory cell. Such cells can include, but are not limited to, bacterial cells, in particular Escherichia coli, yeast cells, such as Saccharomyces cerevisiae, Pichia pastoris, and the like. Such recombinant cells can be used to produce polypeptides and/or encoding nucleic acids of the invention encoding a dominant negative form, which can be isolated or purified, if desired, from said cells using routine molecular biology and protein purification techniques.

The constructs, vectors, transgenes, and immunomodulatory cells described herein can be generated by a method described in or a method similar to the one described in Cherkassky et al., The Journal of Clinical Investigation 126(8):3130-3144 (2016) or the Examples in Section 8.

### 7.5. Methods of Treatment

The invention also relates to methods of treating a mammalian disease or disorder (i.e., a cancer or an infection with a pathogen (such as a viral infection)) using the immunostimulatory cells of the invention. When the immunostimulatory cells as described above are sensitized to a cancer antigen or comprise a CAR that bind to a cancer antigen, such immunostimulatory cells are administered to treat the cancer. When the immunostimulatory cells as described above are sensitized to an antigen of a pathogen or comprise a CAR that bind to an antigen of a pathogen, such immunostimulatory cells are administered to treat an infection with the pathogen.

In specific embodiments, the methods comprise administering a therapeutically effective amount of the immunostimulatory cell described above to a subject having a cancer or an infection with a pathogen (such as a viral infection). In certain embodiments, the target antigen is chosen to target the cancer or infected cells of the subject. The immunostimulatory cells are administered as or as part of a population of cells. Optionally, the population of cells to be administered can be purified or enriched for the immunostimulatory cells of the invention. In a specific embodiment, the invention provides methods of treating a cancer or an infection with a pathogen in a subject in need thereof, comprising administering to the subject a therapeutically effective amount of the immunostimulatory cell described in this disclosure. In another specific embodiment, the invention provides methods of treating a cancer or an infection with a pathogen in a subject in need thereof, comprising administering to the subject a pharmaceutical composition described in Section 7.6, which pharmaceutical composition comprises a therapeutically effective amount of the immunostimulatory cell described in this disclosure and a pharmaceutically acceptable carrier.

In the methods of the invention, the immunostimulatory cells are administered to a subject in need of cancer or pathogen infection treatment. The subject can be a mammal, in particular a human. Preferably, the subject is a human.

The subject can have an advanced form of disease, in which case the treatment objective can include mitigation or reversal of disease progression, and/or amelioration of side effects. The subjects can have a history of the condition, for which they have already been treated, in which case the therapeutic objective can be to decrease or delay the risk of recurrence. Additionally, refractory or recurrent malignancies or infections can be treated using the cells of the invention.

In a specific embodiment of the invention, the immunostimulatory cells that are administered to the subject comprise both CD4⁺ and CD8⁺ T cells, with the aim of generating both helper and cytotoxic T lymphocyte (CTL) responses in the subject.

In one aspect, the methods of the invention can be used to treat cancer or reduce tumor burden in a subject. In one embodiment, the methods of the invention are used to treat cancer. It is understood that a method of treating cancer can include any effect that ameliorates a sign or symptom associated with cancer. Such signs or symptoms include, but are not limited to, reducing tumor burden, including inhibiting growth of a tumor, slowing the growth rate of a tumor, reducing the size of a tumor, reducing the number of tumors, eliminating a tumor, all of which can be measured using routine tumor imaging techniques well known in the art. Other signs or symptoms associated with cancer include, but are not limited to, fatigue, pain, weight loss, and other signs or symptoms associated with various cancers. In one non-limiting example, the methods of the invention can reduce tumor burden. Thus, administration of the cells of the invention can reduce the number of tumor cells, reduce tumor size, and/or eradicate the tumor in the subject. The tumor can be a solid tumor. Non-limiting examples of a solid tumor include mesothelioma, lung cancer, pancreatic cancer, ovarian cancer, breast cancer, colon cancer, pleural tumor, glioblastoma, esophageal cancer, gastric cancer, and synovial sarcoma. The methods of the invention can also provide for increased or lengthened survival of a subject having cancer. Additionally, methods of the invention can provide for an increased immune response in the subject against the cancer.

Suitable human subjects for cancer therapy include those with “advanced disease” or “high tumor burden” who bear a clinically measurable tumor. A clinically measurable tumor is one that can be detected on the basis of tumor mass, for example, by palpation, CAT scan, sonogram, mammogram, X-ray, and the like. Positive biochemical or histopathologic markers can also be used to identify this population. A pharmaceutical composition comprising a cell of the invention is administered to a subject to elicit an anti-cancer response, with the objective of palliating the subject's condition. Reduction in tumor mass of a subject having a tumor can occur, but any clinical improvement constitutes a benefit. Clinical improvement comprises decreased risk or rate of progression or reduction in pathological consequences of the tumor.

Another group of suitable subjects can be a subject who has a history of cancer, but has been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for different types of cancers. Features typical of high-risk subgroups are those in which the tumor has invaded neighboring tissues, or who show involvement of lymph nodes. Optionally, a cell of the invention can be administered for treatment prophylactically to prevent the occurrence of cancer in a subject suspected of having a predisposition to a cancer, for example, based on family history and/or genetic testing.

The cancer can involve a solid tumor or a blood cancer not involving a solid tumor. Cancers to be treated using the cells of the invention comprise cancers typically responsive to immunotherapy. Exemplary types of cancers include, but are not limited to, carcinomas, sarcoma, leukemia, lymphoma, multiple myeloma, melanoma, brain and spinal cord tumors, germ cell tumors, neuroendocrine tumors, carcinoid tumors, and the like. The cancer can be a solid tumor or a blood cancer that does not form a solid tumor. In the case of a solid tumor, the tumor can be a primary tumor or a metastatic tumor.

Examples of other neoplasias or cancers that can be treated using the methods of the invention include bone cancer, intestinal cancer, liver cancer, skin cancer, cancer of the head or neck, melanoma (cutaneous or intraocular malignant melanoma), renal cancer (for example, clear cell carcinoma), throat cancer, prostate cancer (for example, hormone refractory prostate adenocarcinoma), blood cancers (for example, leukemias, lymphomas, and myelomas), uterine cancer, rectal cancer, cancer of the anal region, bladder cancer, brain cancer, stomach cancer, testicular cancer, carcinoma of the fallopian tubes, carcinoma of the endometrium, carcinoma of the cervix, carcinoma of the vagina, carcinoma of the vulva, leukemias (for example, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease, Waldenstrom's macroglobulinemia), cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, heavy chain disease, and solid tumors such as sarcomas and carcinomas, for example, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilm's tumor, cervical cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma.

In one embodiment, the methods of the invention are used to treat a cancer selected from malignant pleural disease, mesothelioma, lung cancer (for example, non-small cell lung cancer), pancreatic cancer, ovarian cancer, breast cancer (for example, metastatic breast cancer, metastatic triple-negative breast cancer), colon cancer, pleural tumor, glioblastoma, esophageal cancer, gastric cancer, and synovial sarcoma. The invention provides therapies that are particularly useful for treating solid tumors, for example, malignant pleural disease, mesothelioma, lung cancer, pancreatic cancer, ovarian cancer, breast cancer, colon cancer, pleural tumor, glioblastoma, esophageal cancer, gastric cancer, and synovial sarcoma. Solid tumors can be primary tumors or tumors in a metastatic state. In the case of a mesothelin directed CAR, mesothelin expressing tumors, include, for example, breast cancer, lung cancer, ovarian cancer, pancreatic cancer, esophagus cancer, colon cancer, gastric cancer, and malignant pleural mesothelioma (MPM).

In another aspect, the methods of the invention can be used to treat an infection with a pathogen (for example, an infection with a virus, a bacterium, a fungus, a protozoan, a helminth, or a protist).

In a specific embodiment, the infection with a pathogen is an infection with a bacterium, such as a mycobacterium or Chlamydia trachomatis.

In another specific embodiment, the infection with a pathogen is an infection with a fungus, such as Cryptococcus neoformans, Pneumocystis jiroveci, a Candida, or an invasive fungus.

In another specific embodiment, the infection with a pathogen is an infection with a protozoan, such as Entamoeba histolytica, Plasmodium, Giardia lamblia, or Trypanosoma brucei.

In another specific embodiment, the infection with a pathogen is an infection with a helminth, such as Ascaris, Trichuris, or hookworm.

In another specific embodiment, the infection with a pathogen is an infection with a protist, such as Toxoplasma gondii.

In another specific embodiment, the infection with a pathogen is an infection with a virus. In a particular embodiment, the viral infection can be, but is not limited to, infection with HIV (e.g., HIV-1 and/or HIV-2), HBV, HCV, HSV, VZV, adenovirus, CMV or EBV. The methods of the invention can be used to treat persistent viral infections, such as latent infections, chronic infections or slow infections, for example, persistent viral infections with HIV, HBV or HCV.

In one particular embodiment, the immunostimulatory cells that are specific to an antigen of a pathogen are isolated from a subject having an infection with the pathogen.

The methods of the invention can be used to reduce or eliminate pathogen load (such as viral load) or a persistent pathogen infection (such as viral infection), such as a chronic, latent or slow pathogen infection (such as viral infection), or to prevent or reduce the severity of relapse or recurrent pathogen infection (such as viral infection).

In certain embodiments wherein the immunostimulatory cells that are administered comprise a nucleotide sequence encoding a dominant negative form of an inhibitor of a cell-mediated immune response, expression of the dominant negative form can promote production of pathogen-specific (such as virus-specific) memory cells. In one particular embodiment, the immunostimulatory cells are made pathogen-specific (such as virus-specific) by expressing a CAR that binds to a pathogen antigen (such as a viral antigen). In one particular embodiment, the immunostimulatory cells that are pathogen-specific (such as virus-specific) are isolated from a subject having a pathogen infection (such as a viral infection). In a particular embodiment, the pathogen-specific (such as virus-specific) immunostimulatory cell is a T cell that recognizes and is sensitized to a pathogen antigen (such as a viral antigen).

The methods of the invention can be used to reduce or eliminate pathogen load (such as viral load) or a persistent pathogen infection (such as viral infection), such as a chronic, latent or slow pathogen infection (such as viral infection), or to prevent or reduce the severity of relapse or recurrent pathogen infection (such as viral infection), by promoting the production of pathogen-specific (such as virus-specific) memory cells.

### 7.5.1 Dosages and Administration

For treatment, the amount administered is an amount effective for producing the desired effect. An effective amount or therapeutically effective amount is an amount sufficient to provide a beneficial or desired clinical result upon treatment. An effective amount can be provided in a single administration or a series of administrations (one or more doses). An effective amount can be provided in a bolus or by continuous perfusion. In terms of treatment, an effective amount is an amount that is sufficient to palliate, ameliorate, stabilize, reverse or slow the progression of the disease, or otherwise reduce the pathological consequences of the disease. The effective amount can be determined by the physician for a particular subject. Several factors are typically taken into account when determining an appropriate dosage to achieve an effective amount. These factors include age, sex and weight of the subject, the condition being treated, the severity of the condition and the form and effective concentration of the cells of the invention being administered.

The cells of the invention are generally administered as a dose based on cells per kilogram (cells/kg) of body weight of the subject to which the cells are administered. Generally the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to 10⁶, depending on the mode and location of administration. In general, in the case of systemic administration, a higher dose is used than in regional administration, where the immunostimulatory cells of the invention are administered in the region of a tumor or infection. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 1×10⁸, 6×10⁴, to 1×10⁸, 7×10⁴ to 1×10⁸, 8×10⁴ to 1×10⁸, 9×10⁴ to 1×10⁸, 1×10⁵ to 1×10⁸, for example, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 3×10⁵ to 3×10⁶ cells/kg, and the like. Such dose ranges can be particularly useful for regional administration. In a particular embodiment, cells are provided in a dose of 1×10⁵ to 1×10⁸, for example 1×10⁵ to 1×10⁷, 1×10⁵ to 1×10⁶, 1×10⁶ to 1×10⁸, 1×10⁶ to 1×10⁷, 1×10⁷ to 1×10⁸, 1×10⁵ to 5×10⁶, in particular 1×10⁵ to 3×10⁶ or 3×10⁵ to 3×10⁶ cells/kg for regional administration, for example, intrapleural administration. Exemplary dose ranges also can include, but are not limited to, 5×10⁵ to 1×10⁸, for example, 6×10⁵ to 1×10⁸, 7×10⁵ to 1×10⁸, 8×10⁵ to 1×10⁸, 9×10⁵ to 1×10⁸, 1×10⁶ to 1×10⁸, 1×10⁶ to 9×10⁷, 1×10⁶ to 8×10⁷, 1×10⁶ to 7×10⁷, 1×10⁶ to 6×10⁷, 1×10⁶ to 5×10⁷, 1×10⁶ to 4×10⁷, 1×10⁶ to 3×10⁷ cells/kg, and the like. Such does can be particularly useful for systemic administration. In a particular embodiment, cells are provided in a dose of 1×10⁶ to 3×10⁷ cells/kg for systemic administration. Exemplary cell doses include, but are not limited to, a dose of 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹ and so forth in the range of about 10⁴ to about 10¹⁰ cells/kg. In addition, the dose can also be adjusted to account for whether a single dose is being administered or whether multiple doses are being administered. The precise determination of what would be considered an effective dose can be based on factors individual to each subject, including their size, age, sex, weight, and condition of the particular subject, as described above. Dosages can be readily determined by those skilled in the art based on the disclosure herein and knowledge in the art.

The cells of the invention can be administered by any methods known in the art, including, but not limited to, pleural administration, intravenous administration, subcutaneous administration, intranodal administration, intratumoral administration, intrathecal administration, intrapleural administration, intraperitoneal administration, intracranial administration, and direct administration to the thymus. In specific embodiments, the administering is by intrapleural administration, intravenous administration, subcutaneous administration, intranodal administration, intratumoral administration, intrathecal administration, intraperitoneal administration, intracranial administration, or direct administration to the thymus. In one embodiment, the cells of the invention can be delivered regionally to a tumor or infection using well known methods, including but not limited to, hepatic or aortic pump; limb, lung or liver perfusion; in the portal vein; through a venous shunt; in a cavity or in a vein that is nearby a tumor or infection, and the like. In another embodiment, the cells of the invention can be administered systemically. In a preferred embodiment, the cells are administered regionally at the site of a tumor or infection. The cells can also be administered intratumorally, for example, by direct injection of the cells at the site of a tumor and/or into the tumor vasculature. For example, in the case of malignant pleural disease, mesothelioma or lung cancer, administration is preferably by intrapleural administration (see Adusumilli et al., Science Translational Medicine 6(261):261ra151 (2014)). One skilled in the art can select a suitable mode of administration based on the type of cancer or infection and/or location of a tumor or infection to be treated. The cells can be introduced by injection or catheter. In one embodiment, the cells are pleurally administered to the subject in need, for example, using an intrapleural catheter. Optionally, expansion and/or differentiation agents can be administered to the subject prior to, during or after administration of cells to increase production of the cells of the invention in vivo.

Proliferation of the cells of the invention is generally done ex vivo, prior to administration to a subject, and can be desirable in vivo after administration to a subject (see Kaiser et al., Cancer Gene Therapy 22:72-78 (2015)). Cell proliferation should be accompanied by cell survival to permit cell expansion and persistence, such as with T cells.

### 7.5.2 Additional Therapies

The methods of the invention can further comprise adjuvant therapy in combination with, either prior to, during, or after treatment with the cells of the invention. Thus, the cell therapy methods of the invention can be used with other standard cancer or infection care and/or therapies that are compatible with administration of the cells of the invention.

Optionally, the methods of administering cells of the invention can additionally include immunomodulation of the host to facilitate the effectiveness of the administered cells of the invention in combination therapy. In an embodiment of the invention, the methods of the invention can further comprise administering at least one pharmaceutical or biological agent that can modulate immune response. Non-limiting examples of pharmaceutical or biological agents that can modulate immune response include immunostimulatory agents, checkpoint immune blockade agents, radiation therapy agents, and chemotherapy agents. In certain embodiments, the pharmaceutical or biological agent that can modulate immune response is an immunostimulatory agent. In one embodiment, the immunostimulatory agent is a cytokine, including but not limited to, IL-2, IL-3, IL-6, IL-7, IL-11, IL-12, IL-15, IL-17, and IL-21. Other exemplary immunostimulatory agents include, but are not limited to, colony stimulating factors, such as G-, M- and GM-CSF, interferons, for example, γ-interferon, and the like. In one embodiment, the methods of the invention further comprise administering IL-2 or GM-CSF to the subject. In a specific embodiment, IL-2 is administered to the subject. The IL-2 or GM-CSF can be administered before, during or after cell therapy using cells of the invention (i.e., concurrently or sequentially), as desired. In a specific embodiment the cytokine (e.g., IL-2 or GM-CSF) is administered on the same day, or during the same week, or within 2 weeks, of the cell therapy using cells of the invention. In a particular embodiment, IL-2 is administered in a dose of about 50,000 to 800,000 international units (IU) per kilogram of body weight, for example, about 50,000 to 720,000, 50,000 to 500,000, 50,000 to 250,000, 50,000 to 200,000, 50,000 to 150,000, 50,000 to 100,000, or about 720,000 IU/kg (Robbins et al., J. Clin. Oncol. 29:917-924 (2011)). In a non-limiting embodiment, IL-2 is administered in a dose of about 50,000, 55,000, 60,000, 61,000, 62,000, 63,000, 64,000, 65,000, 66,000, 67,000, 68,000, 69,000, 70,000, 71,000, 72,000, 73,000, 74,000, 75,000, 76,000, 77,000, 78,000, 79,000, 80,000, 85,000, 90,000, 95,000, 100,000, 110,000, 120,000, 130,000, 140,000, 150,000, 160,000, 170,000, 180,000, 190,000, 200,000, 210,000, 220,000, 230,000, 240,000, 250,000, 260,000, 270,000, 280,000, 290,000, 300,000, 320,000, 340,000, 360,000, 380,000, 400,000, 420,000, 440,000, 460,000, 480,000, 500,000, 520,000, 540,000, 560,000, 580,000, 600,000, 620,000, 640,000, 660,000, 680,000, 700,000, 720,000, 740,000, 760,000, 780,000 or 800,000 IU/kg. Given the improved efficacy of immunostimulatory cell therapy using cells of the invention, it is expected that the doses of cytokines, such as IL-2, suitable as combination therapy with a cell of the invention can be lower than that used with other therapies using cytokines. Administering a cytokine, for example, IL-2 or GM-CSF, is particularly useful if the CAR expressed in the immunostimulatory cell results in reduced expression of an immunostimulatory cell stimulatory cytokine, such as IL-2 or GM-CSF. The cytokine can be administered to enhance the efficacy of the immunostimulatory cells of the invention expressing the CAR and dominant negative form. As described in Cherkassky et al., The Journal of Clinical Investigation 126(8):3130-3144 (2016), T cells expressing a PD-1 dominant negative form and MBBz CAR, having 4-1BB as a co-stimulatory signaling domain, exhibit decreased expression of IL-2, whereas T cells expressing a PD-1 dominant negative form and M28z CAR, having CD28 as a co-stimulatory signaling domain, have increased expression of IL-2. Accordingly, the invention provides for treating cancer in a subject having cancer by administering to the subject T cells expressing a PD-1 dominant negative form and a MBBz CAR, which CAR has 4-1BB as a co-stimulatory signaling domain, and administering to the subject IL-2. A person skilled in the art can readily assay an immunostimulatory cell of the invention for expression of immunostimulatory cytokines and, if desired, optionally administer an immunostimulatory cytokine that is deficiently expressed by the cells to a subject being treated with the cells. Such a combination therapy including an immunostimulatory cytokine can be used to increase the efficacy of immunostimulatory cell therapy using such cells, for example, cells expressing a dominant negative form of an immune checkpoint inhibitor with reduced immunostimulatory cytokine production.

Additional immunostimulatory agents include agonist costimulatory monoclonal antibodies, such as anti-4-1BB antibodies, anti-0×40 antibodies, and anti-ICOS antibodies. In one embodiment, the agonist costimulatory monoclonal antibody is an anti-4-1BB antibody.

Among all immunotherapeutic approaches, IL-12, a multifunctional cytokine, has been considered to be one of the most promising approaches to treat breast cancer (Boggio et al., Cancer Res. 60:359-364 (2000); Czerniecki et al., Cancer Res. 67:1842-1852 (2007); Nanni et al., J. Exp. Med. 194:1195-1205 (2001)). IL-12 is considered a master regulator of adaptive type 1 cell-mediated immunity, the critical pathway involved in antitumor responses (Del Vecchio et al., Clin. Cancer Res. 13:4677-4685 (2007)). IL-12 modulates antitumor responses at various levels, including polarization of CD4 T cells toward a Thl phenotype (Wesa et al., J. Immunother. 30, 75-82 (2007)), boosting of T cell and NK effector functions (Curtsinger et al., J. Exp. Med. 197:1141-1151 (2003)), remodeling the innate immune response (Chmielewski et al., Cancer Res. 71:5697-5706 (2011)), and regulating tumor angiogenesis (Voest et al., J. Natl. Cancer. Inst. 87:581-586 (1995)). Among 148 clinical trials including administration of IL-12 to patients with cancer, successful phase II studies with intraperitoneal (Lenzi et al., Clin. Cancer Res. 8:3686-3695 (2002); Lenzi et al., J. Transl. Med. 5:66 (2007)) or subcutaneous (Mahvi et al., Cancer Gene Ther. 14:717-723 (2007); Kang et al., Hum. Gene Ther. 12:671-684 (2001)) IL-12 have shown that paracrine secretion of IL-12, generated by gene transfer, can induce immunity against the tumor locally and at a distant site. Although several studies have documented the anticancer effectiveness of IL-12 in preclinical models of breast cancer (Boggio et al., Cancer Res. 60:359-364 (2000); Nanni et al., J. Exp. Med. 194:1195-1205 (2001); Brunda et al., J. Exp. Med. 178:1223-1230 (1993)), the significant toxicity resulting from administration of recombinant human IL-12 observed in several clinical trials in advanced cancers precludes its clinical use. To overcome this limitation, a number of groups have demonstrated that intratumoral delivery of IL-12, using adenoviral vectors, induces tumor regression and T cell activation in preclinical models of breast cancer (Gyorffy et al., J. Immunol. 166:6212-6217 (2001); Bramson et al., Hum. Gene Ther. 7:1995-2002 (1996)). More recently, polylactic acid microspheres were used to release IL-12 into the tumor, and it was found that the antitumor response was mediated primarily by NK cells (Sabel et al., Breast Cancer Res. Treat. 122:325-336 (2010)). Others have used mesenchymal stromal cells to locally deliver IL-12 to mouse breast cancer (Eliopoulos et al., Cancer Res. 68, 4810-4818 (2008)). A phase I trial of paclitaxel and trastuzumab, in combination with IL-12, in patients with HER2/neu-expressing malignancies showed an impressive synergy between IL-12 and trastuzumab for stimulation of NK-cell cytokine secretion (Bekaii-Saab et al., Mol. Cancer Ther. 8:2983-2991 (2009)). Therefore, IL-12 is particularly useful as an anticancer agent to be used as a co-stimulant in an adoptive immune cell therapy approach, including some embodiments of the methods of the invention disclosed herein. The immunomodulating and antiangiogenic functions of IL-12 support the use of this cytokine in combination with a cell of some embodiments of the invention for treating cancers.

In another embodiment, the pharmaceutical or biological agent that can modulate immune response is a co-stimulatory ligand. Co-stimulatory ligands include, without limitation, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD4OL/CD154, CD137L/4-1BBL, TNF-α, CD134L/OX4OL/CD252, CD27L/CD70, Fas ligand (FasL), CD3OL/CD153, tumor necrosis factor beta (TNFβ)/lymphotoxin-alpha (LTα), lymphotoxin-beta (LTβ), CD257/B cell-activating factor (BAFF)/Blys/THANK/Tall-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins, that is, they possess an immunoglobulin domain (fold). Immunoglobulin superfamily ligands include, without limitation, CD80 and CD86, both ligands for CD28. In some embodiments, the at least one co-stimulatory ligand is selected from the group consisting of 4-1BBL, CD80, CD86, CD70, OX40L, CD48, TNFRSF14, and the like.

In another embodiment, the pharmaceutical or biological agent that can modulate immune response can be an immune checkpoint blockade agent. The administration of an immune checkpoint blockade agent supplements the inhibition of immune checkpoint blockade provided by expressing a dominant negative form of an immune checkpoint inhibitor in a cell of the invention. Non-limiting examples of immune checkpoint blockade agents include anti-PD-L1 antibodies, anti-CTLA-4 antibodies, anti-PD-1 antibodies, anti-LAG3 antibodies, anti-B7-H3 antibodies, anti-TIM3 antibodies, and the like. Such immune checkpoint blockade agents include, but are not limited to, antibodies to PD-1, CTLA-4, BTLA, TIM-3, LAG-3, CD160, TIGIT, LAIR1, 2B4, and the like, or antibodies to the corresponding ligands for these receptors including, for example, PD-L1 (for PD-1); PD-L2 (for PD-1); CD80, CD86 (for CTLA-4); HVEM (for BTLA); Galectin-9, HMGB1 (for TIM-3); MHC II (for LAG-3); HVEM (for CD160); CD155, CD112, CD113 (for TIGIT); C1q, collagen (for LAIR1); CD48 (for 2B4), and the like. In one embodiment, the checkpoint immune blockade agent is an anti-PD-L1 antibody. It is understood that an antibody that inhibits the activity of an immune checkpoint inhibitor by binding to the immune checkpoint inhibitor receptor or its corresponding ligand, including receptors and ligands as disclosed herein, can be used as pharmaceutical or biological agent that can modulate immune response to further suppress the immunoinhibitory effect in an immunostimulatory cell of the invention expressing a dominant negative form. In a particular embodiment, the antibody will be to the immune checkpoint inhibitor, or its ligand, that corresponds to the dominant negative form being expressed in the immunostimulatory cell of the invention, which can be useful to further suppress any residual activity in the immunostimulatory cell expressing the dominant negative form. In certain embodiments, the methods of the invention can optionally include administration of an immune checkpoint blockade agent such as antibodies directed to the ligand and/or receptor of an immune checkpoint pathway.

In some embodiments, the pharmaceutical or biological agent that can modulate immune response can be a radiation therapy agent. The localized, radiation-induced immunological milieu can provide the preconditions to enhance the engraftment of cells of the invention at the site of the tumor, thereby eliminating the need for systemic lymphodepleting regimens. The immunological responses resulting from a combination of radiation therapy, particularly low dose radiation therapy, and cell therapy methods of the invention also can enhance abscopal antitumor efficacy. In some embodiments, the pharmaceutical or biological agent that can modulate immune response is a chemotherapy agent, including, but not limited to, cisplatin, cyclophosphamide, and the like. Cisplatin-induced secretion of chemokines and cytokines can promote cancer antigen-targeted cells of the invention and endogenous immune cell responses such as T-cell responses. Cyclophosphamide can function as a lymphodepleting agent, for example, as a preparatory lymphodepleting agent.

Tumor irradiation- and cisplatin therapy-induced tumoral and abscopal immunomodulation can provide the preconditioning required for better engraftment of cells of the invention.

Co-stimulatory strategies, as described above, can potentiate the antitumor or anti-infection efficacy of both endogenous T cells and the cells of the invention.

Optionally, a cell of the invention can express a co-stimulatory receptor (CCR) that binds to an antigen different than the target antigen (see Sadelain, et al., Cancer Discovery 3(4):388-398 (2013), Chicaybam, et al., Int. Rev. Immunol. 30(5-6):294-311 (2011), Brentjens et al., Nature Medicine 9:279- 286 (2003); U.S. 7,446,190 and U.S. 2013/0071414 (CD19-targeted CARs); Ahmed, et al., Clin. Cancer Res. 16(2):474-485(2010)(HER2-targeted CARs); Chekmasova, et al., Clin. Cancer Res. 16(14):3594-606 (2010)(MUC16-targeted CARs); Zhong, et al., Molecular Therapy, 18(2):413-420 (2010) and U.S. Pat. No. 7,446,190 (prostate-specific membrane antigen (PSMA)-targeted CARs), all of which are herein incorporated by reference. CCRs mimic co-stimulatory signals but, unlike CARs, do not provide a T cell activation signal (see Sadelain, et al., Cancer Discovery 3(4):388-398 (2013)). Immune cells expressing two or more antigen recognizing receptors are described in WO 2014/055668, which is herein incorporated by reference.

Administering a pharmaceutical or biological agent that can modulate immune response in a combination therapy with an immunostimulatory cell of the invention can occur concurrently with administration of the immunostimulatory cells of the invention, for example, when immunostimulatory cell therapy is initiated, or can occur sequentially at any time during the immunostimulatory cell therapy, as desired. A person skilled in the art can readily determine appropriate regimens for administering cells of the invention and a pharmaceutical or biological agent that can modulate immune response in a combination therapy, including the timing and dosing of an immunomodulatory agent to be used in a combination therapy, based on the needs of the subject being treated.

### 7.6. Pharmaceutical Compositions

The invention additionally provides pharmaceutical compositions comprising the cells of the invention. The pharmaceutical composition comprises a therapeutically effective amount of an immunostimulatory cell of the invention and a pharmaceutically acceptable carrier. The cells of the invention and compositions comprising the cells can be conveniently provided in sterile liquid preparations, for example, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the invention in a suitable amount of the appropriate solvent with various amounts of the other ingredients, as desired. Such compositions can include a pharmaceutically acceptable carrier, diluent, or excipient such as sterile water, physiological saline, glucose, dextrose, or the like, that are suitable for use with a cell composition and for administration to a subject such as a human. Suitable buffers for providing a cell composition are well known in the art. Any vehicle, diluent, or additive used is compatible with preserving the integrity and viability of the cells of the invention.

The compositions will generally be isotonic, that is, they have the same osmotic pressure as blood and lacrimal fluid. The desired isotonicity of the cell compositions of the invention can be accomplished using sodium chloride, or other pharmaceutically acceptable agents such as dextrose, boric acid, sodium tartrate, or other inorganic or organic solutes. Sodium chloride is preferred particularly for buffers containing sodium ions. One particularly useful buffer is saline, for example, normal saline. Those skilled in the art will recognize that the components of the compositions should be selected to be chemically inert and will not affect the viability or efficacy of the cells of the invention and will be compatible for administration to a subject, such as a human. The skilled artisan can readily determine the amount of cells and optional additives, vehicles, and/or carrier in compositions to be administered in methods of the invention.

The cells of the invention can be administered in any physiologically acceptable vehicle. Suitable doses for administration are described herein. A cell population comprising cells of the invention can comprise a purified population of cells. Those skilled in the art can readily determine the percentage of cells in a cell population using various well-known methods, as described herein. The ranges of purity in cell populations comprising genetically modified cells of the invention can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art; for example, a decrease in purity may require an increase in dosage.

The invention also provides kits for preparation of cells of the invention. In a specific embodiment, the kit comprises one or more vectors as described herein in one or more containers. In one embodiment, the kit comprises one or more vectors for generating a genetically engineered immunostimulatory cell, such as a T cell, described herein. The kits can be used to generate genetically engineered immunostimulatory cells from autologous cells derived from a subject or from non-autologous cells to be administered to a compatible subject. In another embodiment, the kits can comprise cells of the invention, for example, autologous or non-autologous cells, for administration to a subject. In specific embodiments, the kits comprise the immunostimulatory cells of the invention in one or more containers.

It is understood that modifications which do not substantially affect the activity of the various embodiments of this invention are also provided within the definition of the invention provided herein. Accordingly, the following example is intended to illustrate but not limit the present invention.

### 8. EXAMPLES

Certain embodiments provided herein are illustrated by the following non-limiting examples, which describe the generation of immunostimulatory cells of the invention for use in treatment of a human disease.

### 8.1. Example 1

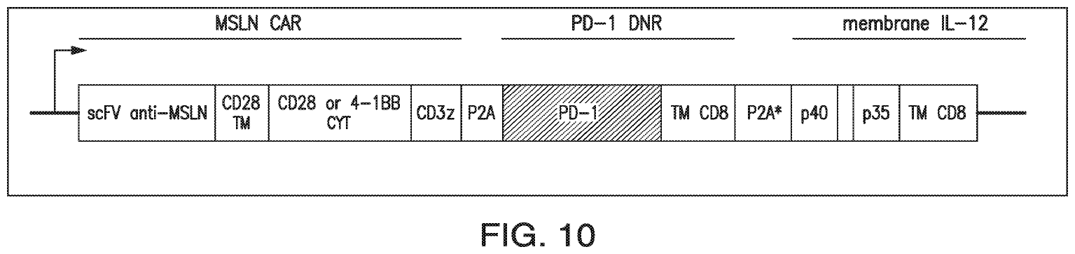

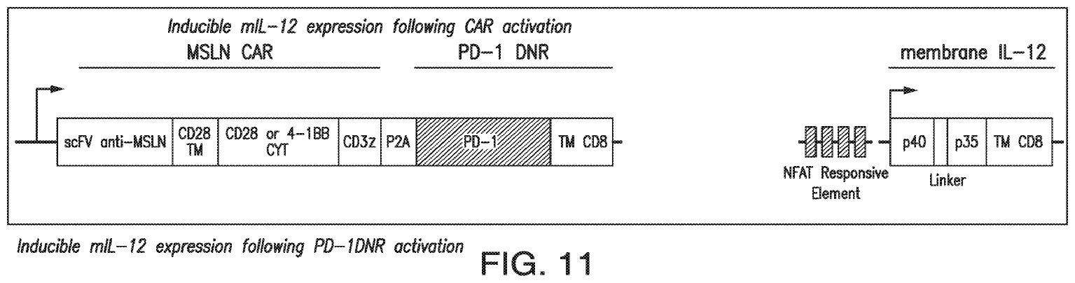

Although clinical trials with administration of IL-12 have shown anti-tumor modulation of the tumor microenvironment, the toxicity of secreted IL-12 has been prohibitive. Therefore, the constructs shown in FIGS. 10-11 were designed such that transduced T cells, constitutively or upon T cell activation (resulting in NFAT induction of transcription) will express only membrane IL-12, which will bind to the IL-12R on cells to elicit beneficial anti-tumor immune responses, but will not secrete IL-12, thereby avoiding toxicity.

To make the constructs illustrated in FIGS. 10-11, mesothelin-specific CARs were first generated by engineering a fusion protein encoding a fully human scFv, m912, ligated to a human CD8 leader peptide at its N-terminus. Using γ-retroviral vectors as backbone constructs, this scFv was exchanged to generate second generation (SFG-M28z or SFG-MBBz) mesothelin-specific constructs by directional cloning using a Ncol site located 5′ of the scFv and a Notl site located 3′ of the scFv. An internal ribosomal entry site was inserted to facilitate bicistronic expression of CARs with a reporter gene (for example, LNGFR). SFG-M28z or MBBz was then transfected into 293T H29 packaging cell lines and the viral supernatant was used to transduce and generate stable 293T RD114 cell lines.

For construction of the dominant negative form of PD-1, the extracellular portion of the PD-1 receptor was fused to the CD8 transmembrane and hinge domains using the SFG γ-retroviral vector. The dominant negative PD-1 encoding plasmid was transfected into 293T H29 and 293VecRD114 packaging cell lines to produce the retrovirus.

The above constructs were utilized to induce the expression of membrane IL-12 (mIL-12) in an inducible or constitutive manner using the SFG retroviral vector. The mIL-12 was generated by fusion of CD8 transmembrane domain to the single chain IL-12 cytokine (p40 and p35 subunits separated by a peptide linker).

A multicistronic vector was generated containing the MSLN-CAR, the dominant negative PD-1 and mIL-12 constructs separated by P2A peptide self cleavage motif under control of a constitutive promoter, to constitutively express the three proteins in CAR T cells (see FIG. 10). T cells were transduced with the multicistronic vector to express the MSLN-CAR, the dominant negative PD-1 and mIL-12, which were expressed by the T cells.

A bicistronic vector was generated with MSLN-CAR and the dominant negative PD-1 constructs separated by P2A peptide self cleavage motif under control of a constitutive promoter, to constitutively express the two proteins in CAR T cells (see FIG. 11). Another vector was generated to express mIL-12 under the control of NFAT promoter (see FIG. 11). T cells were co-transduced with both vectors, to constitutively express the MSLN-CAR and dominant negative PD-1 and inducibly express mIL-12 under the control of NFAT promoter (see FIG. 11), which were expressed by the T cells.

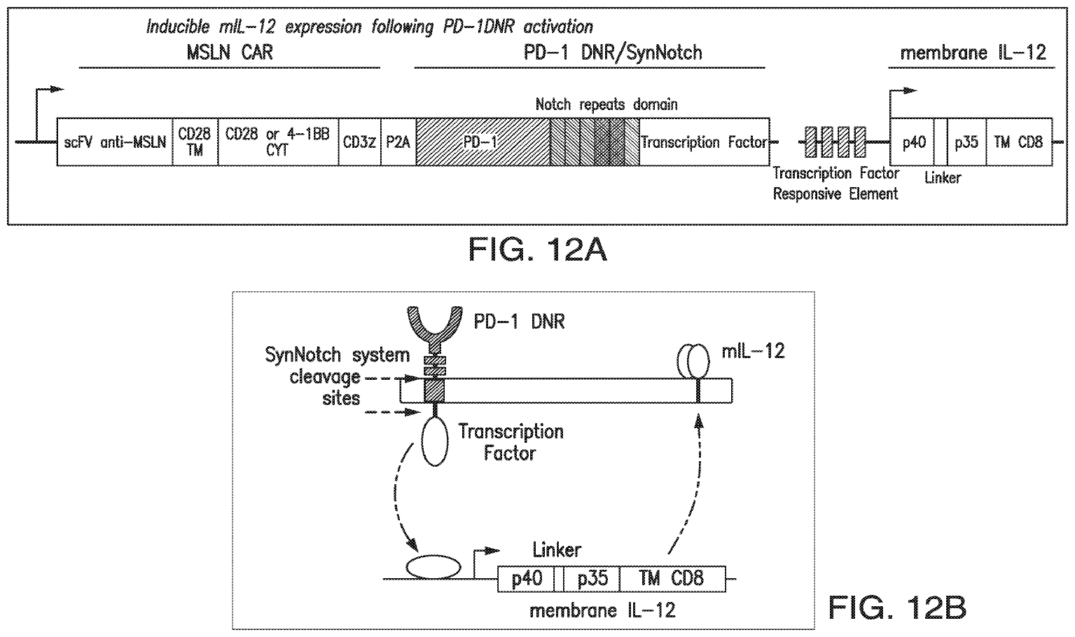

The constructs illustrated in FIGS. 1-4, 6-9, and 12-14 were also made. In particular, for the construct illustrated in FIG. 12A, the dominant negative PD-1 receptor-synthetic Notch fusion protein was generated by fusing the cDNA encoding for PD-1 extracellular domain to the Notch regulatory cleavage region and the Gal4-VP64 transcription factor, and the expression of membrane IL-12 was under the control of a Gal4 promoter.

### 8.2. Example 2

Construction of Vectors and Generation of T Cells

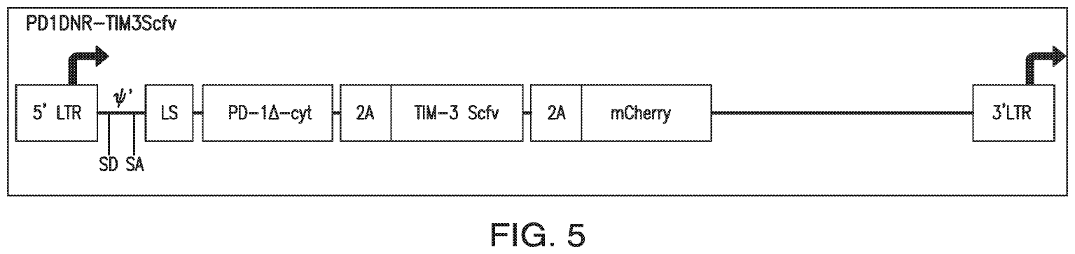

For each of the constructs illustrated in FIGS. 5 and 15-18, the different corresponding nucleotide sequence elements are engineered into the SFG γ-retroviral vector (provided by I. Riviere, MSKCC). The MSLN-specific CAR sequence and the dominant negative form of PD-1 sequence are generated as described in Example 1.

Vectors containing the constructs illustrated in FIGS. 1-9 and 12-18 are each transfected into 293T H29 packaging cell lines and the viral supernatants are used to transduce and generate stable 293T RD114 cell lines to produce the retrovirus, as previously described (Hollyman et al., J. Immunother. 32(2):169-180 (2009)).

Peripheral blood leukocytes are isolated from the blood of healthy volunteer donors under an institutional review board—approved protocol. Peripheral blood mononuclear cells (PBMCs) are isolated by low-density centrifugation on Lymphoprep (Stem Cell Technology, Vancouver, Canada) and activated with phytohemagglutinin (2 μg/mL; Remel, Lenexa, KS). Two days after isolation, PBMCs are transduced with retroviral particles produced as described above and spinoculated for 1 h at 3000 rpm on plates coated with retronectin (15 μg/mL; r-Fibronectin, Takara, Tokyo, Japan). After 1 day, transduced PBMCs are maintained in IL-2 (20 UI/mL; Novartis, Basel, Switzerland). Vector-expressing T cells are isolated by flow cytometry and expanded in vitro.

For vectors containing constructs illustrated in FIGS. 1-4, and 15-18, after transduction of T cells with these vectors, the dominant negative form of PD-1 or the dominant negative form of TGF-β receptor along with either TIM-3 scFv (scFv that bind to TIM-3) or LAG-3 scFv (scFv that bind to LAG-3) are expressed constitutively. While expression of dominant negative form of PD-1 or TGF-β receptor on the T cell surface should help to increase functional persistence of that particular T cell (due to binding of the dominant negative form of PD-1 to PD-L1/PD-L2, or binding of the dominant negative form of TGF-β receptor to TGF-β), secreted TIM-3 scFv or LAG-3 scFv should help neutralize their corresponding ligands in the tumor microenvironment, thereby preventing inhibition of activated T cells, which are activated upon CAR activation by recognition of mesothelin expressed on cancer cell surface. Constitutive expression of the dominant negative form of PD-1 on the T cell can help modulate the tumor microenvironment beyond the T cell itself by binding to PD-L1/PD-L2 expressed on non-antigen expressing cancer cells as well as immune cells.

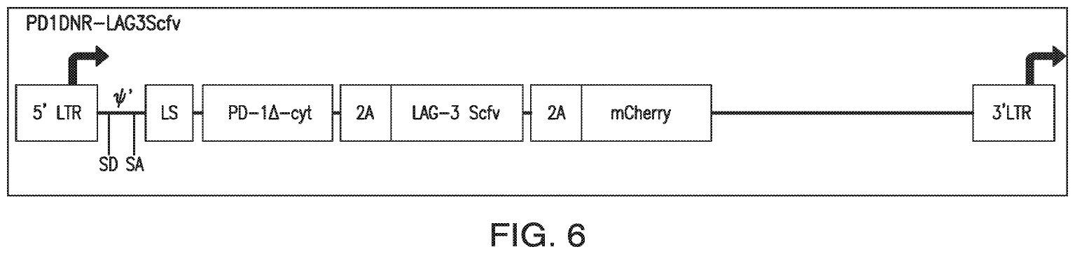

For vectors containing the constructs illustrated in FIGS. 5-6, after transduction of T cells with these vectors, the vectors constitutively express the dominant negative form of PD-1 along with TIM-3 scFv, or LAG-3 scFv, in the T cells. The dominant negative form of PD-1 as well as secreted scFvs should modulate the tumor microenvironment in the same way as described above.

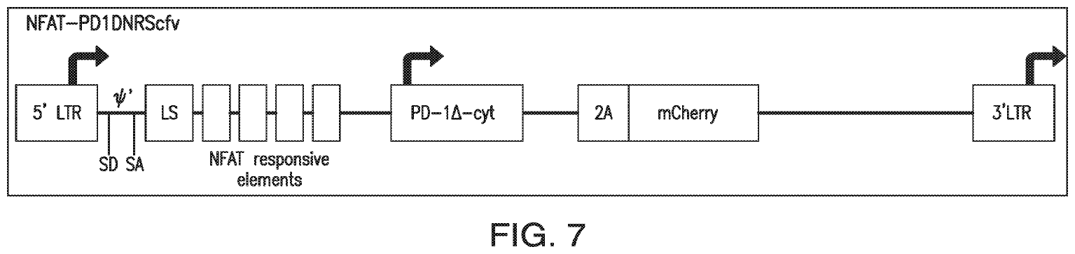

For vectors containing the constructs illustrated in FIGS. 7-9, after transduction of T cells with these vectors, the vectors express the dominant negative form of PD-1, TIM-3 scFv, or LAG-3 scFv, only upon T cell activation (resulting in NFAT induction of transcription).

For vectors containing the construct illustrated in FIG. 12A, after transduction of T cells with this vector, the receptor-synthetic Notch fusion protein allows expression of membranous IL-12 only upon engagement of the dominant negative form of PD-1 to PD-L1/PD-L2 (see FIG. 12B), thereby concentrating the membranous IL-12 within the tumor microenvironment only.

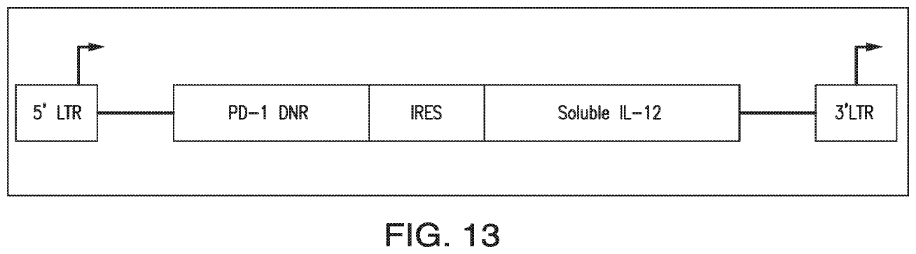

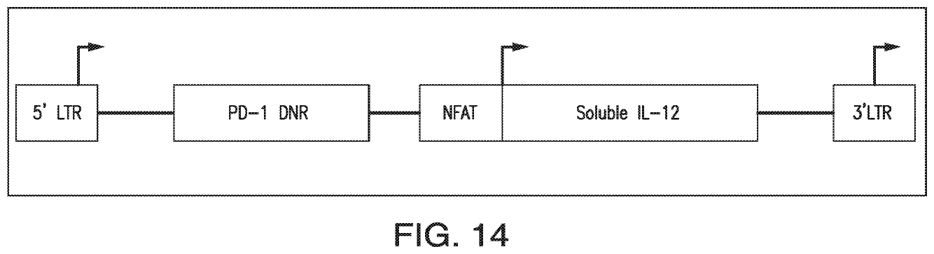

For vectors containing the constructs illustrated in FIG. 13-14, after transduction of T cells with these vectors, the vectors express secreted IL-12 either constitutively or upon T cell activation (resulting in NFAT induction of transcription).

Treatment of Cancer

A human patient presents with pleural mesothelioma. A population of T cells generated as described above is selected for administration. The patient receives 2 ×10⁶ cells per kilogram of body weight by intrapleural administration. The patient is monitored before, during, and after the T cell administration for clinical response.

### 9. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A T cell comprising in one or more transgenes: (a) a first nucleotide sequence encoding a dominant negative form of an inhibitor of a cell-mediated immune response of the T cell, and (b) a second nucleotide sequence encoding an immunomodulatory agent, wherein the immunomodulatory agent is a single chain variable fragment (scFv) or peptide antibody, which immunomodulatory agent binds to and inhibits an immune checkpoint inhibitor, and wherein the immune checkpoint inhibitor is different from the inhibitor of a cell-mediated immune response.

2. The T cell of claim 1, wherein the dominant negative form of the inhibitor of a cell-mediated immune response is expressed as a membrane protein on the T cell surface.

3. The T cell of claim 1 or 2, wherein the inhibitor of a cell-mediated immune response is an immune checkpoint inhibitor.

4. The T cell of claim 3, wherein the inhibitor of a cell-mediated immune response is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160.

5. The T cell of claim 4, wherein the inhibitor of a cell-mediated immune response is PD-1.

6. The T cell of claim 1 or 2, wherein the inhibitor of a cell-mediated immune response is TGF-β receptor.

7. The T cell of any one of claims 1-6, wherein the immunomodulatory agent is secreted from the T cell.

8. The T cell of any one of claims 1-7, wherein the immunomodulatory agent is a scFv.

9. The T cell of any one of claims 1-7, wherein the immunomodulatory agent is a peptide antibody.

10. The T cell of any one of claims 1-9, wherein the immune checkpoint inhibitor to which the immunomodulatory agent binds is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160.

11. The T cell of claim 10, wherein the immune checkpoint inhibitor to which the immunomodulatory agent binds is TIM-3.

12. The T cell of claim 10, wherein the immune checkpoint inhibitor to which the immunomodulatory agent binds is LAG-3.

13. The T cell of any one of claims 1-12, which comprises a transgene comprising (a) the first nucleotide sequence encoding a dominant negative form of an inhibitor of a cell-mediated immune response of the T cell, and (b) the second nucleotide sequence encoding an immunomodulatory agent, wherein a nucleotide sequence encoding a cleavable linker is present in between the first nucleotide sequence encoding a dominant negative form and the second nucleotide sequence encoding an immunomodulatory agent, and wherein expression of the transgene is under control of a promoter such that the transgene is expressible in the T cell to produce the dominant negative form and the immunomodulatory agent.

14. The T cell of claim 13, wherein the promoter is constitutive.

15. The T cell of claim 13 or 14, wherein the transgene further comprises a third nucleotide sequence encoding a reporter, wherein a nucleotide sequence encoding a cleavable linker is present in between any adjacent occurrences in the transgene of the first nucleotide sequence encoding a dominant negative form of an inhibitor of a cell-mediated immune response of the T cell, the second nucleotide sequence encoding an immunomodulatory agent, and the third nucleotide sequence encoding a reporter, and wherein the transgene is expressible in the T cell to produce the reporter.

16. The T cell of any one of claims 1-15, wherein the T cell recognizes and is sensitized to a target antigen associated with a mammalian disease or disorder.

17. The T cell of any one of claims 1-12, wherein the T cell further comprises a fourth nucleotide sequence encoding a chimeric antigen receptor (CAR), wherein the CAR binds to a target antigen that is associated with a mammalian disease or disorder.

18. The T cell of claim 13 or 14, wherein the transgene further comprises a fourth nucleotide sequence encoding a chimeric antigen receptor (CAR), wherein the CAR binds to a target antigen that is associated with a mammalian disease or disorder, and wherein a nucleotide sequence encoding a cleavable linker is present in between any adjacent occurrences in the transgene of the first nucleotide sequence encoding a dominant negative form of an inhibitor of a cell-mediated immune response of the T cell, the second nucleotide sequence encoding an immunomodulatory agent, and the fourth nucleotide sequence encoding a CAR, and wherein the transgene is expressible in the T cell to produce the CAR.

19. The T cell of claim 15, wherein the transgene further comprises a fourth nucleotide sequence encoding a chimeric antigen receptor (CAR), wherein the CAR binds to a target antigen that is associated with a mammalian disease or disorder, and wherein a nucleotide sequence encoding a cleavable linker is present in between any adjacent occurrences in the transgene of the first nucleotide sequence encoding a dominant negative form of an inhibitor of a cell-mediated immune response of the T cell, the second nucleotide sequence encoding an immunomodulatory agent, the third nucleotide sequence encoding a reporter, and the fourth nucleotide sequence encoding a CAR, and wherein the transgene is expressible in the T cell to produce the CAR.

20. A T cell comprising a transgene, which transgene comprises a first nucleotide sequence encoding a dominant negative form of an inhibitor of a cell-mediated immune response of the T cell, wherein expression of the transgene is under control of an inducible promoter, which inducible promoter is induced upon activation of the T cell.

21. The T cell of claim 20, wherein the inducible promoter is induced by nuclear factor of activated T cells (NFAT) binding.

22. The T cell of claim 20 or 21, wherein the dominant negative form of the inhibitor of a cell-mediated immune response is expressed as a membrane protein on the T cell surface.

23. The T cell of any one of claims 20-22, wherein the inhibitor of a cell-mediated immune response is an immune checkpoint inhibitor.

24. The T cell of claim 23, wherein the inhibitor of a cell-mediated immune response is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160.

25. The T cell of claim 24, wherein the inhibitor of a cell-mediated immune response is PD-1.

26. The T cell of any one of claims 20-22, wherein the inhibitor of a cell-mediated immune response is TGF-β receptor.

27. The T cell of any one of claims 20-26, wherein the transgene further comprises a second nucleotide sequence encoding a reporter, wherein a nucleotide sequence encoding a cleavable linker is present in between the first nucleotide sequence encoding a dominant negative form of an inhibitor of a cell-mediated immune response of the T cell and the second nucleotide sequence encoding a reporter, and wherein the transgene is expressible in the T cell to produce the reporter.

28. The T cell of any one of claims 20-27, wherein the T cell recognizes and is sensitized to a target antigen associated with a mammalian disease or disorder.

29. A T cell comprising a transgene, which transgene comprises a first nucleotide sequence encoding an immunomodulatory agent, wherein expression of the transgene is under control of an inducible promoter, which inducible promoter is induced upon activation of the T cell, wherein the immunomodulatory agent is a single chain variable fragment (scFv) or peptide antibody, which immunomodulatory agent binds to and inhibits an immune checkpoint inhibitor.

30. The T cell of claim 29, wherein the inducible promoter is induced by nuclear factor of activated T cells (NFAT) binding.

31. The T cell of claim 29 or 30, wherein the immunomodulatory agent is secreted from the T cell.

32. The T cell of any one of claims 29-31, wherein the immunomodulatory agent is a scFv.

33. The T cell of any one of claims 29-31, wherein the immunomodulatory agent is a peptide antibody.

34. The T cell of any one of claims 29-33, wherein the immune checkpoint inhibitor to which the immunomodulatory agent binds is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160.

35. The T cell of claim 34, wherein the immune checkpoint inhibitor to which the immunomodulatory agent binds is TIM-3.

36. The T cell of claim 34, wherein the immune checkpoint inhibitor to which the immunomodulatory agent binds is LAG-3.

37. The T cell of any one of claims 29-36, wherein the transgene further comprises a second nucleotide sequence encoding a reporter, wherein a nucleotide sequence encoding a cleavable linker is present in between the first nucleotide sequence encoding an immunomodulatory agent and the second nucleotide sequence encoding a reporter, and wherein the transgene is expressible in the T cell to produce the reporter.

38. The T cell of any one of claims 29-37, wherein the T cell recognizes and is sensitized to a target antigen associated with a mammalian disease or disorder.

39. The T cell of any one of claims 16-19, 28, and 38, wherein the mammalian disease or disorder is a cancer and the target antigen is a cancer antigen.

40. The T cell of claim 39, wherein the cancer antigen is selected from the group consisting of mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-a and _{R} (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), K-light chain, kinase insert domain receptor (KDR), Lewis A (CA19.9), Lewis Y (LeY), L1 cell adhesion molecule (LlCAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF- R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Ra, Latent Membrane Protein 1 (LMP1), Neural cell-adhesion molecule (N-CAM/CD56), and Trail Receptor (TRAIL R).

41. The T cell of claim 40, wherein the cancer antigen is mesothelin.

42. The T cell of any one of claims 16-19, 28, and 38, wherein the mammalian disease or disorder is an infection with a pathogen and the target antigen is an antigen of the pathogen.

43. The T cell of claim 42, wherein the pathogen is a human pathogen.

44. The T cell of claim 42 or 43, wherein the pathogen is a virus, a bacterium, a fungus, a protozoan, a helminth, or a protist.

45. The T cell of any one of claims 42-44, wherein the target antigen is a viral antigen.

46. The T cell of claim 45, wherein the viral antigen can elicit an immune response in a human subject infected with the virus.

47. The T cell of claim 45 or 46, wherein the viral antigen is selected from the group consisting of a human immunodeficiency virus (HIV) antigen, a hepatitis B virus (HBV) antigen, a hepatitis C virus (HCV) antigen, a herpes simplex virus (HSV) antigen, a varicella zoster virus (VZV) antigen, an adenovirus antigen, a cytomegalovirus (CMV) antigen, and an Epstein-Barr virus (EBV) antigen.

48. The T cell of claim 47, wherein the viral antigen is a HIV antigen selected from the group consisting of group-specific antigen (gag) protein, p55, p24, p18, envelope glycoprotein (env), gp160, gp120, gp41, reverse transcriptase (pol), p66, and p31.

49. The T cell of claim 47, wherein the viral antigen is a HBV antigen selected from the group consisting of HBV envelope protein S, HBV envelope protein M, HBV envelope protein L, and the S domain of HBV envelope protein S, M or L.

50. The T cell of claim 47, wherein the viral antigen is a HCV antigen selected from the group consisting of core protein, envelope protein E1, envelope protein E2, NS2, NS3, NS4, and NSS.

51. The T cell of claim 47, wherein the viral antigen is a HSV antigen selected from the group consisting of gE, gI, gB, gD, gH, gL, gC, gG, gK, gM, and the extracellular domain of gE.

52. The T cell of claim 47, wherein the viral antigen is a VZV antigen selected from the group consisting of gE and gI.

53. The T cell of claim 47, wherein the viral antigen is an adenovirus antigen selected from the group consisting of hexon protein and penton protein.

54. The T cell of claim 47, wherein the viral antigen is a CMV antigen selected from the group consisting of pp65, immediate early (IE) antigen, and IEl.

55. The T cell of claim 47, wherein the viral antigen is an EBV antigen selected from the group consisting of latent membrane protein 2 (LMP2), Epstein-Barr nuclear antigen 1 (EBNA1), and BZLF1.

56. The T cell of any one of claims 1-55, wherein the T cell further recombinantly expresses a suicide gene.

57. The T cell of claim 56, wherein the suicide gene comprises inducible Caspase 9.

58. The T cell of any one of claims 1-57, wherein the T cell is a cytotoxic T lymphocyte (CTL).

59. The T cell of any one of claims 1-57, wherein the T cell is CD4⁺.

60. The T cell of any one of claims 1-57, wherein the T cell is CD8⁺.

61. The T cell of any one of claims 1-60, wherein the T cell is derived from a human.

62. An immunostimulatory cell comprising in one or more transgenes: (a) a first nucleotide sequence encoding a chimeric antigen receptor (CAR), wherein the CAR binds to a target antigen associated with a mammalian disease or disorder, (b) a second nucleotide sequence encoding a dominant negative form of an inhibitor of a cell-mediated immune response of the immunostimulatory cell, and (c) a third a nucleotide sequence encoding a membrane bound form of interleukin 12 (membrane IL-12).

63. The immunostimulatory cell of claim 62, which comprises a transgene comprising: (a) the first nucleotide sequence encoding a chimeric antigen receptor (CAR), (b) the second nucleotide sequence encoding a dominant negative form of an inhibitor of a cell-mediated immune response of the immunostimulatory cell, and (c) the third nucleotide sequence encoding a membrane bound form of interleukin 12 (membrane IL-12), wherein a nucleotide sequence encoding a cleavable linker is present in between any adjacent occurrences in the transgene of the first nucleotide sequence encoding a CAR, the second nucleotide sequence encoding a dominant negative form of an inhibitor of a cell-mediated immune response of the immunostimulatory cell, and the third nucleotide sequence encoding a membrane IL-12, and wherein expression of the transgene is under control of a promoter such that the transgene is expressible in the immunostimulatory cell to produce the CAR, the dominant negative form and the membrane IL-12.

64. The immunostimulatory cell of claim 63, wherein the promoter is constitutive.

65. The immunostimulatory cell of claim 62, which comprises: (1) a first transgene, which first transgene comprises: (a) the first nucleotide sequence encoding a chimeric antigen receptor (CAR), and (b) the second nucleotide sequence encoding a dominant negative form of an inhibitor of a cell-mediated immune response of the immunostimulatory cell, and (2) a second transgene, which second transgene comprises (c) the third nucleotide sequence encoding a membrane bound form of interleukin 12 (membrane IL-12), wherein a nucleotide sequence encoding a cleavable linker is present in between the first nucleotide sequence encoding a CAR and the second nucleotide sequence encoding a dominant negative form of an inhibitor of a cell-mediated immune response of the immunostimulatory cell, and wherein expression of the first transgene is under control of a promoter such that the first transgene is expressible in the immunostimulatory cell to produce the CAR and the dominant negative form, and wherein expression of the second transgene is under control of an inducible promoter, which inducible promoter is induced upon activation of the immunostimulatory cell.

66. The immunostimulatory cell of claim 65, wherein the promoter is constitutive.

67. The immunostimulatory cell of claim 62, which comprises: (1) a first transgene, which first transgene comprises (a) the nucleotide sequence encoding a chimeric antigen receptor (CAR), (2) a second transgene, which second transgene comprises (b) the nucleotide sequence encoding a dominant negative form of an inhibitor of a cell-mediated immune response of the immunostimulatory cell, and (3) a third transgene, which third transgene comprises (c) the nucleotide sequence encoding a membrane bound form of interleukin 12 (membrane IL-12), wherein expression of the third transgene is under control of an inducible promoter, which inducible promoter is induced upon activation of the immunostimulatory cell.

68. The immunostimulatory cell of claim 67, wherein the first and the second transgenes are under control of a constitutive promoter.

69. The immunostimulatory cell of any one of claims 65-68, wherein the inducible promoter is induced by nuclear factor of activated T cells (NFAT) binding.

70. An immunostimulatory cell comprising: (1) in one or more transgenes: (a) a first nucleotide sequence encoding a chimeric antigen receptor (CAR), wherein the CAR binds to a target antigen associated with a mammalian disease or disorder, (b) a second nucleotide sequence encoding a dominant negative form of an inhibitor of a cell-mediated immune response of the immunostimulatory cell, that is a receptor-synthetic Notch fusion protein comprising (i) an extracellular domain of the inhibitor of a cell-mediated immune response of the immunostimulatory cell, (ii) the transmembrane core domain of Notch C-terminal to the extracellular domain, and (iii) a transcription factor C-terminal to the transmembrane core domain of Notch, and (2) in a different transgene (c) a third nucleotide sequence encoding a membrane bound form of interleukin 12 (membrane IL-12), wherein expression of the membrane IL-12 is under control of an inducible promoter, which inducible promoter is induced upon binding of the transcription factor, and wherein the transcription factor is cleaved from the receptor-synthetic Notch fusion protein intracellularly upon binding of the extracellular domain to its ligand.

71. The immunostimulatory cell of claim 70, which comprises: (1) a first transgene, which first transgene comprises: (a) the first nucleotide sequence encoding a chimeric antigen receptor (CAR), and (b) the second nucleotide sequence encoding a receptor-synthetic Notch fusion protein , and (2) a second transgene, which second transgene comprises (c) the third nucleotide sequence encoding a membrane IL-12, wherein a nucleotide sequence encoding a cleavable linker is present in between the first nucleotide sequence encoding a CAR and the second nucleotide sequence encoding a receptor-synthetic Notch fusion protein, and wherein expression of the first transgene is under control of a promoter such that the first transgene is expressible in the immunostimulatory cell to produce the CAR and the receptor-synthetic Notch fusion protein, and wherein expression of the second transgene is under control of an inducible promoter, which inducible promoter is induced upon binding of the transcription factor, and wherein the transcription factor is cleaved from the receptor-synthetic Notch fusion protein intracellularly upon binding of the extracellular domain to its ligand.

72. The immunostimulatory cell of claim 71, wherein the promoter is constitutive.

73. The immunostimulatory cell of claim 70, which comprises: (1) a first transgene, which first transgene comprises: (a) the first nucleotide sequence encoding a chimeric antigen receptor (CAR), (2) a second transgene, which second transgene comprises: (b) the second nucleotide sequence encoding a receptor-synthetic Notch fusion protein, and (3) a third transgene, which third transgene comprises (c) the third nucleotide sequence encoding a membrane IL-12, wherein the first and second transgenes are expressible in the immunostimulatory cell to produce the CAR and the receptor-synthetic Notch fusion protein, and wherein expression of the second transgene is under control of an inducible promoter, which inducible promoter is induced upon binding of the transcription factor, and wherein the transcription factor is cleaved from the receptor-synthetic Notch fusion protein intracellularly upon binding of the extracellular domain to its ligand.

74. The immunostimulatory cell of claim 73, wherein the first and second transgenes are under control of constitutive promoters.

75. The immunostimulatory cell of any one of claims 62-74, wherein the membrane IL-12 comprises a p40 subunit and a p35 subunit separated by a linker, and wherein the p35 subunit is fused to a transmembrane domain.

76. An immunostimulatory cell comprising in one or more transgenes: (a) a first nucleotide sequence encoding a dominant negative form of an inhibitor of a cell-mediated immune response of the immunostimulatory cell, and (b) a second nucleotide sequence encoding interleukin 12 (IL-12), wherein the IL-12 when expressed by the immunostimulatory cell is secreted from the immunostimulatory cell.

77. The immunostimulatory cell of claim 76, which comprises a transgene comprising: (a) the first nucleotide sequence encoding a dominant negative form of an inhibitor of a cell-mediated immune response of the immunostimulatory cell, and (b) the second nucleotide sequence encoding interleukin 12 (IL-12), wherein the first nucleotide sequence encoding the dominant negative form and the second nucleotide sequence encoding the IL-12 are separated by an internal ribosome entry site (IRES), wherein expression of the transgene is under control of a promoter such that the transgene is expressible in the immunostimulatory cell to produce the dominant negative form and the IL-12.

78. The immunostimulatory cell of claim 77, wherein the promoter is constitutive.

79. The immunostimulatory cell of claim 76, which comprises: (1) a first transgene comprising (a) the first nucleotide sequence encoding a dominant negative form of an inhibitor of a cell-mediated immune response of the immunostimulatory cell, and (2) a second transgene comprising (b) the second nucleotide sequence encoding interleukin 12 (IL-12), wherein expression of the dominant negative form is under control of a promoter such that the first transgene is expressible in the immunostimulatory cell to produce the dominant negative form, wherein expression of the IL-12 is under control of an inducible promoter, which inducible promoter is induced upon activation of the immunostimulatory cell, and wherein the IL-12 when expressed by the immunostimulatory cell is secreted from the immunostimulatory cell.

80. The immunostimulatory cell of claim 79, wherein the promoter is constitutive.

81. The immunostimulatory cell of claim 79 or 80, wherein the inducible promoter is induced by nuclear factor of activated T cells (NFAT) binding.

82. The immunostimulatory cell of any one of claims 76-81, wherein the immunostimulatory cell recognizes and is sensitized to a target antigen associated with a mammalian disease or disorder.

83. The immunoinhibitory cell of any one of claims 62-75 and 82, wherein the mammalian disease or disorder is a cancer and the target antigen is a cancer antigen.

84. The immunostimulatory cell of claim 83, wherein the cancer antigen is selected from the group consisting of mesothelin, prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (LlCAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF- R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Ra, Latent Membrane Protein 1 (LMP1), Neural cell-adhesion molecule (N-CAM/CD56), and Trail Receptor (TRAIL R).

85. The immunostimulatory cell of claim 84, wherein the cancer antigen is mesothelin.

86. The immunostimulatory cell of any one of claims 62-75 and 82, wherein the mammalian disease or disorder is an infection with a pathogen and the target antigen is an antigen of the pathogen.

87. The immunostimulatory cell of claim 88, wherein the pathogen is a human pathogen.

88. The immunostimulatory cell of claim 86 or 87, wherein the pathogen is a virus, a bacterium, a fungus, a protozoan, a helminth, or a protist.

89. The immunostimulatory cell of claim 88, wherein the target antigen is a viral antigen.

90. The immunostimulatory cell of claim 89, wherein the viral antigen can elicit an immune response in a human subject infected with the virus.

91. The immunostimulatory cell of claim 89 or 90, wherein the viral antigen is selected from the group consisting of a human immunodeficiency virus (HIV) antigen, a hepatitis B virus (HBV) antigen, a hepatitis C virus (HCV) antigen, a herpes simplex virus (HSV) antigen, a varicella zoster virus (VZV) antigen, an adenovirus antigen, a cytomegalovirus (CMV) antigen, and an Epstein-Barr virus (EBV) antigen.

92. The immunostimulatory cell of claim 91, wherein the viral antigen is a HIV antigen selected from the group consisting of group-specific antigen (gag) protein, p55, p24, p18, envelope glycoprotein (env), gp160, gp120, gp41, reverse transcriptase (pol), p66, and p31.

93. The immunostimulatory cell of claim 91, wherein the viral antigen is a HBV antigen selected from the group consisting of HBV envelope protein S, HBV envelope protein M, HBV envelope protein L, and the S domain of HBV envelope protein S, M or L.

94. The immunostimulatory cell of claim 91, wherein the viral antigen is a HCV antigen selected from the group consisting of core protein, envelope protein E1, envelope protein E2, NS2, NS3, NS4, and NS5.

95. The immunostimulatory cell of claim 91, wherein the viral antigen is a HSV antigen selected from the group consisting of gE, gI, gB, gD, gH, gL, gC, gG, gK, gM, and the extracellular domain of gE.

96. The immunostimulatory cell of claim 91, wherein the viral antigen is a VZV antigen selected from the group consisting of gE and gl.

97. The immunostimulatory cell of claim 91, wherein the viral antigen is an adenovirus antigen selected from the group consisting of hexon protein and penton protein.

98. The immunostimulatory cell of claim 91, wherein the viral antigen is a CMV antigen selected from the group consisting of pp65, immediate early (IE) antigen, and IEl.

99. The immunostimulatory cell of claim 91, wherein the viral antigen is an EBV antigen selected from the group consisting of latent membrane protein 2 (LMP2), Epstein-Barr nuclear antigen 1 (EBNA1), and BZLF1.

100. The immunostimulatory cell of any one of claims 62-99, wherein the dominant negative form of the inhibitor of a cell-mediated immune response is expressed as a membrane protein on the immunostimulatory cell surface.

101. The immunostimulatory cell of any one of claims 62-100, wherein the inhibitor of a cell-mediated immune response is an immune checkpoint inhibitor.

102. The immunostimulatory cell of claim 101, wherein the inhibitor of a cell-mediated immune response is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160.

103. The immunostimulatory cell of claim 102, wherein the inhibitor of a cell-mediated immune response is PD-1.

104. The immunostimulatory cell of any one of claims 62-100, wherein the inhibitor of a cell-mediated immune response is TGF-β receptor.

105. The immunostimulatory cell of any one of claims 62-104, wherein the immunostimulatory cell is a T cell.

106. The immunostimulatory cell of claim 105, wherein the T cell is a cytotoxic T lymphocyte (CTL).

107. The immunostimulatory cell of claim 105, wherein the T cell is CD4⁺.

108. The immunostimulatory cell of claim 105, wherein the T cell is CD8⁺.

109. The immunostimulatory cell of any one of claims 62-104, wherein the immunostimulatory cell is a Natural Killer (NK) cell.

110. The immunostimulatory cell of any one of claims 62-109, wherein the immunostimulatory cell further recombinantly expresses a suicide gene.

111. The immunostimulatory cell of claim 110, wherein the suicide gene comprises inducible Caspase 9.

112. The immunostimulatory cell of any one of claims 62-111, wherein the immunostimulatory cell is derived from a human.

113. A pharmaceutical composition comprising a therapeutically effective amount of the T cell of any one of claims 1-41, or of any one of claims 56-61 when dependent directly or indirectly on any one of claims 1-41, or the immunostimulatory cell of any one of claims 62-85, or of any one of claims 100-112 when dependent directly or indirectly on any one of claims 62-85.

114. A pharmaceutical composition comprising a therapeutically effective amount of the T cell of any one of claims 1-38 and 42-55, or of any one of claims 56-61 when dependent directly or indirectly on any one of claims 1-38 and 42-55, or the immunostimulatory cell of any one of claims 62-82 and 86-99, or of any one of claims 100-112 when dependent directly or indirectly on any one of claims 62-82 and 86-99.

115. A method of treating a cancer in a subject in need thereof, comprising administering to the subject a therapeutically effective amount of the T cell of any one of claims 1-41, or of any one of claims 56-61 when dependent directly or indirectly on any one of claims 1-41, or the immunostimulatory cell of any one of claims 62-85, or of any one of claims 100-112 when dependent directly or indirectly on any one of claims 62-85.

116. A method of treating an infection with a pathogen in a subject in need thereof, comprising administering to the subject a therapeutically effective amount of the T cell of any one of claims 1-38 and 42-55, or of any one of claims 56-61 when dependent directly or indirectly on any one of claims 1-38 and 42-55, or the immunostimulatory cell of any one of claims 62-82 and 86-99, or of any one of claims 100-112 when dependent directly or indirectly on any one of claims 62-82 and 86-99.

117. A method of treating a cancer in a subject in need thereof, comprising administering to the subject the pharmaceutical composition of claim 113.

118. A method of treating an infection with a pathogen in a subject in need thereof, comprising administering to the subject the pharmaceutical composition of claim 114.

119. The method of claim 115 or 117, wherein the cancer is selected from the group consisting of mesothelioma, lung cancer, pancreatic cancer, ovarian cancer, breast cancer, colon cancer, pleural tumor, glioblastoma, esophageal cancer, gastric cancer, and synovial sarcoma.

120. The method of claim 116 or 119, wherein the infection with a pathogen is an infection with a virus, a bacterium, a fungus, a protozoan, a helminth, or a protist.

121. The method of claim 120, wherein the infection with a pathogen is an infection with a virus.

122. The method of claim 121, wherein the infection with a pathogen is an infection with HCV, HIV, HBV, HSV, VZV, adenovirus, CMV or EBV.

123. The method of any one of claims 115-122, wherein the subject is a human.

124. The method of any one of claims 115-123, wherein the administering is by intrapleural administration, intravenous administration, subcutaneous administration, intranodal administration, intratumoral administration, intrathecal administration, intraperitoneal administration, intracranial administration, or direct administration to the thymus.

125. The method of any one of claims 115-124, wherein the T cell or the immunostimulatory cell is administered in a dose in the range of 10⁴ to 10¹⁰ cells per kilogram of body weight.

126. The method of claim 125, wherein the dose is in the range of 3×10⁵ to 3×10⁶ cells per kilogram of body weight.
